(19) United States
(12) Patent Application Publication
(10) Pub. No.: US 2016/0367578 A1
(43) Pub. Date: Dec. 22, 2016

(54) METHODS AND COMPOSITIONS FOR ERADICATING LEUKEMIC CELLS

(71) Applicants: President and Fellows of Harvard College, Cambridge, MA (US); The General Hospital Corporation, Boston, MA (US)

(72) Inventors: Amir Schajnovitz, Cambridge, MA (US); David T. Scadden, Weston, MA (US)

(21) Appl. No.: 15/130,623

(22) Filed: Apr. 15, 2016

Related U.S. Application Data
(63) Continuation-in-part of application No. PCT/US2014/060734, filed on Oct. 15, 2014.
(60) Provisional application No. 61/891,259, filed on Oct. 15, 2013.

(51) Int. Cl. A61K 31/704; A61K 45/06; A61K 31/215

(52) U.S. Cl. CPC A61K 31/704; A61K 31/215; A61K 45/06

(57) ABSTRACT

The disclosure relates to compositions, methods, and kits comprising glycyrrhetinic acid derivatives for selectively eradicating leukemic cells in a population or subject, and related methods of treating acute myeloid leukemia, and promoting survival of acute myeloid leukemia patients.

### RELATED APPLICATIONS

This application is a continuation-in-part application of International Application No. PCT/US2014/060734, filed Oct. 15, 2014, which claims the benefit of U.S. Provisional Application Ser. No. 61/891,259, filed Oct. 15, 2013, the teachings of which are incorporated herein by reference in their entirety.

### GOVERNMENT SUPPORT

This invention was made with government support under R01HL097794 awarded by the National Institutes of Health. The government has certain rights in the invention.

### BACKGROUND OF THE INVENTION

Acute myeloid leukemia (AML) is a genetically heterogeneous disease of blood stem and myeloid progenitor cells, characterized by the accumulation of malignant blasts in the bone marrow that severely impairs normal blood formation. In spite of the heterogeneous nature of AML, the various subtypes seem to share some common pathways leading to leukemogenesis, and the hierarchical nature of the disease is generally well established (Lane, et al., Blood 1150-1157 (2009)). AML is one of the best characterized malignancies from a genetic viewpoint. Numerous genetic transformation events leading to leukemia have been characterized (Marcucci, et al., J. Clinical Oncology 29, 475-486 (2011); Pui, et al., J. Clinical Oncology 29, 551-65 (2011); and Burnett, et al., J. Clinical Oncology 29, 487-94 (2011)). In addition to cell-autonomous events, reciprocal interactions of the leukemic cells with the microenvironment has been reported (Gillette, et al., Nature Cell Biology 11, 303-11 (2009); Walkley, et al., Cell 129, 1097-110 (2007); and Wei, et al., Cancer Cell 13, 483-95 (2008)), suggesting a consequential cross-talk between the leukemic cells and the microenvironment. However, despite the progress in cataloging the molecular alterations involved in leukemogenesis, our understanding of how such changes cooperate to induce drug resistance is still weak. Moreover, very little is known regarding a whole stratum of junctional interactions between leukemic cells in general and during induction chemotherapy in particular.

### SUMMARY OF THE INVENTION

In certain aspects, the inventions disclosed herein related to methods of eradicating leukemic cells in a population of cells, the method comprising contacting the population of cells with an effective amount of an agent (e.g., carbenoxolone), thereby eradicating leukemic cells in the cell population. In certain embodiments, disclosed herein are methods of treating acute myeloid leukemia in a subject in need thereof, the method comprising administering to the subject an effective amount of an agent (e.g., a glycyrrhetinic acid derivative), thereby treating acute myeloid leukemia in the subject. Also disclosed herein are methods of promoting survival of a subject suffering from acute myeloid leukemia, the method comprising administering to the subject an effective amount of an agent (e.g., a glycyrrhetinic acid derivative), and thereby promoting survival of the subject. In some embodiments, the inventions disclosed herein relate to methods of promoting the differentiation of a leukemic cell into a non-leukemic cell, the method comprising contacting the leukemic cell with an effective amount of an agent (e.g., 18-β-glycyrrhetinic acid), thereby promoting the differentiation of the leukemic cell into a non-leukemic cell.

In certain aspects, the agents for use in accordance with the inventions disclosed herein comprise a glycyrrhetinic acid derivative. Exemplary glycyrrhetinic acid derivatives include glycyrrhizine, glycyrrhizinic acid, 18-β-glycyrrhetinic acid, carbenoxolone, 2-hydroxyethyl-18β-glycyrrhetinic acid amide and analogs thereof. In some embodiments, the agent is or comprises 18-β-glycyrrhetinic acid or a derivative thereof. In some embodiments, the agent is or comprises carbenoxolone or an analog thereof. In some aspects, the agent is or comprises a gap junction blocker. In some embodiments, the agent is or comprises a hemichannel blocker. In still other embodiments, the agent is or comprises a blocker or inhibitor of one or more of connexins, pannexins and/or hydroxysteroid dehydrogenase.

In an aspect, the disclosure provides a method of eradicating leukemic cells in a population of cells, the method comprising contacting the population of cells with an effective amount of a gap junction blocker, thereby eradicating leukemic cells in the cell population. In some aspects, the disclosure provides a method of eradicating leukemic cells in a population of cells, the method comprising contacting the population of cells with an effective amount of a hemichannel blocker, thereby eradicating leukemic cells in the cell population.

In some embodiments, the agent or gap junction blocker comprises an inhibitor of 11β-hydroxysteroid dehydrogenase (1β-HSD). In some embodiments, the agent or gap junction blocker is selected from the group consisting of the following

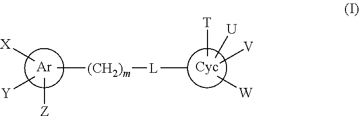

formulas I to III wherein X₁ Y and Z each independently represent halogen, in particular, F, Cl, I or Br, C₁-C₆ alkyl, C₅-C₁₅ aryl or C₁-C₆ alkoxy, n represents an integer from 1 to 10, in particular, from 1 to 4, L represents an amide, amine, sulfonamide, ester, thioester or keto group, T, U, V and W each independently represent an oxo, thio, ketone, thioketone, C₁-C₆ alkyl or C₁-C₆ alkanol group, Ar represents an aromatic ring system, and Cyc represents a cyclic ring system,

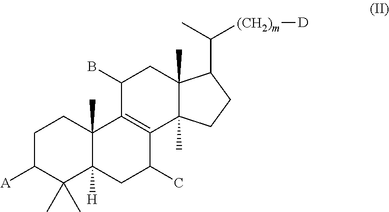

wherein A represents a C₁-C₁₀ ester (C₁-C₁₀ alkyl-CO—O—), a C₁-C₁₀ amide (C₁-C₁₀ alkyl-CO—NH—), a C₁-C₁₀ ether or a C₁-C₁₀ ketone (C₁-C₁₀ alkyl-CO—) group, B and C each independently represent an oxo group, a keto group, a C₁-C₆ alkanol group or a C₁-C₆ alkyl group, m is an integer from 1 to 10, in particular, from 1 to 4, and D is a group selected from COOR¹ or CONR²R³, wherein R¹, R² and R³ each independently represent H or a C₁-C₆ alkyl group,

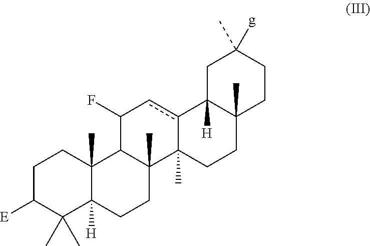

wherein E represents an OH, a C₁-C₁₀ ester (C₁-C₁₀ alkyl-CO—O—), a C₁-C₁₀ amide (C₁-C₁₀ alkyl-CO—NH—), a C₁-C₁₀ ether (C₁-C₁₀—O—) or a C₁-C₁₀ ketone (C₁-C₁₀ alkyl-CO—) group, F represents an oxo group, keto group, a C₁-C₆ alkanol group or a C₁— C₆ alkyl group, and G is a group selected from COOR¹ or CONR²R³, wherein R¹, R² and R³ each independently represent H or a C₁-C₂₀ hydrocarbon group, in particular, a C₁-C₆ alkyl group.

In an aspect, the disclosure provides a method of eradicating leukemic cells in a population of cells, the method comprising contacting the population of cells with an effective amount of an agent or gap junction blocker, thereby eradicating leukemic cells in the cell population. In some embodiments, the agent or gap junction blocker is 18-β-glycyrrhetinic acid or a derivative thereof. In some embodiments, the agent or gap junction blocker is a derivative of 18-β-glycyrrhetinic acid is selected from the group consisting of glycyrrhizine, glycyrrhizinic acid, carbenoxolone or 2-hydroxyethyl-18β-glycyrrhetinic acid amide. In some embodiments, the agent or gap junction blocker comprises carbenoxolone or an analog thereof. In some embodiments, the agent or gap junction blocker is not 18-β-glycyrrhetinic acid. In some embodiments, the agent or gap junction blocker is selected from the group consisting of heptanol octanol, anadamide, fenamate, retinoic acid, oleamide, spermine, aminosulphates, halothane, enflurane, isoflurane, propofol, thiopental, glycyrrhetinic acid, quinine, 2-aminoethoxydiphenyl borate or a pharmaceutically acceptable derivatives thereof, and any combination thereof. In some embodiments, the pharmaceutically acceptable derivatives comprise: a pharmaceutically acceptable derivative of heptanol selected from the group consisting of I-heptanol, 2-heptanol, 3-heptanol, 4-heptanol, and combinations thereof; a pharmaceutically acceptable derivative of fenamate selected from the group consisting of meclofenamic acid, niflumic acid, flufenamic acid, and combinations thereof; a pharmaceutically acceptable derivative of glycyrrhetinic acid selected from the group consisting of hydrogen esters of glycyrrhetinic acid, salts of hydrogen esters of glycyrrhetinic acid, carbenoxolone, and combinations thereof; and a pharmaceutically acceptable derivative of quinine selected from the group consisting of quinidine, mefloquine, and combinations thereof.

In some embodiments, eradicating leukemic cells comprises inducing the differentiation of the leukemic cells into granulocytes. In some embodiments, the granulocytes comprise neutrophils. In some embodiments, the neutrophils comprise CD66b+/CD14-neutrophils. In some embodiments, eradicating leukemic cells comprises disrupting intercellular communications involving the leukemic cells that promote leukemia cell survival. In some embodiments, disrupting intercellular communications involving leukemic cells comprises interfering with heterotypic interactions between leukemic cells and stromal cells (e.g., mesenchymal stromal cells). In some embodiments, disrupting intercellular communications involving leukemic cells comprises interfering with heterotypic interactions between leukemic cells and any other cell types (e.g., osteolineage cells, endothelial cells, pericytes, mesenchymal cells or other hematopoietic cells). In some embodiments, disrupting intercellular communications involving leukemic cells comprises interfering with homotypic interactions between leukemic cells. In some embodiments, the leukemic cells are selectively eradicated while inducing proliferation of normal leukocytes in the population of cells. In some embodiments, the leukemic cells are selectively eradicated without eradicating normal leukocytes cells in the population of cells. In some embodiments, at least 20% of the leukemic cells in the population of cells are eradicated. In some embodiments, at least 50% of the leukemic cells in the population of cells are eradicated. In some embodiments, at least 70% of the leukemic cells in the population of cells are eradicated. In some embodiments, all of the leukemic cells in the population of cells are eradicated.

In some embodiments, the leukemic cells are selectively eradicated, while minimally eradicating normal leukocytes cells in the population of cells. In some embodiments, the leukemic cells are selectively eradicated by carbenoxolone (e.g., at a concentration of carbenoxolone of about 5 μM, 10 μM, 25 μM, 50 μM, 100 μM, 200 μM, 250 μM or 500 μM), while minimally eradicating normal leukocytes cells in the population of cells. For example, in certain aspect the leukemic cells are selectively eradicated (e.g., at a concentration of carbenoxolone of about 50 μM), while less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001% or less of the normal cells are eradicated. In certain aspects, carbenoxolone selectively eradicates stem cells (e.g., leukemic stem cells) in the population of cells, relative non-stem cells in the population of cells.

In some embodiments, the leukemic cells comprise an acute myeloid leukemia cell line selected from the group consisting of MLL-AF9 cells, MLL-ENL cells, Nup98-HoxA9 cells, AML1-ET09A cells, KG-1 cells, KG-1a cells, U937 cells, HL60 cells, NB-4 cells, HoxA9/Meis1 cells, and THP1 cells.

In some embodiments, the population of cells comprises primary leukocytes selected from the group consisting of bone marrow leukocytes and peripheral blood leukocytes.

In some embodiments, the effective amount comprises a concentration in the range of 50 μM to 400 μM in vitro or 10 mg/kg to 100 mg/kg in vivo.

In some embodiments, the contacting occurs in vitro or ex vivo. In some embodiments, the contacting occurs in vivo. In some embodiments, the in vivo contact is in a subject. In some embodiments, the subject is a mouse. In some embodiments, the subject is a human. In some embodiments, the subject suffers from leukemia. In some embodiments, the subject suffers from acute myeloid leukemia.

In aspect, the disclosure provides a method of promoting the differentiation of a leukemic cell into a non-leukemic cell, the method comprising contacting the leukemic cell with an effective amount of an agent or a gap junction blocker, thereby promoting the differentiation of the leukemic cell into a non-leukemic cell.

In some embodiments, the leukemic cell comprises a leukemic stem or progenitor cell. In some embodiments, the leukemic stem or progenitor cell comprises an acute myeloid leukemia cell. In some embodiments, the acute myeloid leukemia comprises a cell line selected from the group consisting of MLL-AF9 cells, MLL-ENL cells, Nup98-HoxA9 cells, AML1-ET09A cells, KG-1 cells, KG-1a cells, U937 cells, HL60 cells, NB-4 cells, HoxA9/Meis1 cells, and THP1 cells.

In some embodiments, the non-leukemic cell comprises a mature or terminally differentiated cell. In some embodiments, the non-leukemic cell comprises a granulocyte. In some embodiments, the granulocyte comprises a short-lived granulocyte. In some embodiments, the non-leukemic cell comprises a neutrophil. In some embodiments, the neutrophil comprises a CD66b+/CD14− neutrophil.

In some embodiments, the agent or gap junction blocker comprises an inhibitor of 11β-hydroxysteroid dehydrogenase (11β-HSD). In some embodiments, the agent or gap junction blocker is selected from the group consisting of the following formulas I to III:

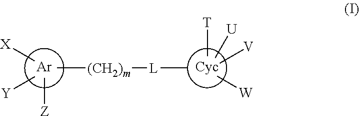

wherein X₁ Y and Z each independently represent halogen, in particular, F, Cl, I or Br, C₁-C₆ alkyl, C₅-C₁₅ aryl or C₁-C₆ alkoxy, n represents an integer from 1 to 10, in particular, from 1 to 4, L represents an amide, amine, sulfonamide, ester, thioester or keto group, T, U, V and W each independently represent an oxo, thio, ketone, thioketone, C₁-C₆ alkyl or C₁-C₆ alkanol group, Ar represents an aromatic ring system, and Cyc represents a cyclic ring system,

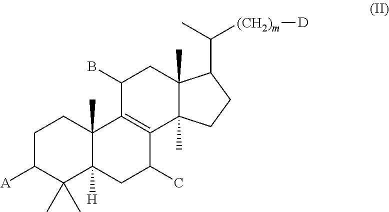

wherein A represents a C₁-C₁₀ ester (C₁-C₁₀ alkyl-CO—O—), a C₁-C₁₀ amide (C₁-C₁₀ alkyl-CO—NH—), a C₁-C₁₀ ether or a C₁-C₁₀ ketone (C₁-C₁₀ alkyl-CO—) group, B and C each independently represent an oxo group, a keto group, a C₁-C₆ alkanol group or a C₁-C₆ alkyl group, m is an integer from 1 to 10, in particular, from 1 to 4, and D is a group selected from COOR¹ or CONR²R³, wherein R¹, R² and R³ each independently represent H or a C₁-C₆ alkyl group,

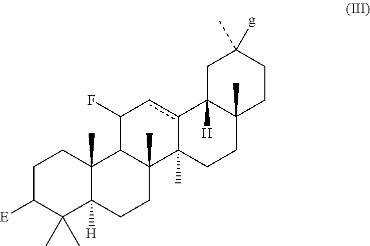

wherein E represents an OH, a C₁-C₁₀ ester (C₁-C₁₀ alkyl-CO—O—), a C₁-C₁₀ amide (C₁-C₁₀ alkyl-CO—NH—), a C₁-C₁₀ ether (C₁-C₁₀—O—) or a C₁-C₁₀ ketone (C₁-C₁₀ alkyl-CO—) group,
F represents an oxo group, keto group, a C₁-C₆ alkanol group or a C₁-C₆ alkyl group, and G is a group selected from COOR¹ or CONR²R³, wherein R¹, R² and R³ each independently represent H or a C₁-C₂₀ hydrocarbon group, in particular, a C₁-C₆ alkyl group. In some embodiments, the agent or gap junction blocker is 18-β-glycyrrhetinic acid or a derivative thereof. In some embodiments, the agent or gap junction blocker is a derivative of 18-β-glycyrrhetinic acid is selected from the group consisting of glycyrrhizine, glycyrrhizinic acid, carbenoxolone or 2˜hydroxyethyl-18β-glycyrrhetinic acid amide. In some embodiments, the agent of gap junction blocker comprises carbenoxolone or an analog thereof. In some embodiments, the agent or gap junction blocker is selected from the group consisting of heptanol, octanol, anadamide, fenamate, retinoic acid, oleamide, spermine, aminosulphates, halothane, enflurane, isoflurane, propofol, thiopental, glycyrrhetinic acid, quinine, 2-aminoethoxydiphenyl borate or a pharmaceutically acceptable derivatives thereof, and any combination thereof.

In some embodiments, the pharmaceutically acceptable derivatives comprise: a pharmaceutically acceptable derivative of heptanol selected from the group consisting of 1-heptanol, 2-heptanol, 3-heptanol, 4-heptanol, and combinations thereof; a pharmaceutically acceptable derivative of fenamate selected from the group consisting of meclofenamic acid, niflumic acid, flufenamic acid, and combinations thereof; a pharmaceutically acceptable derivative of glycyrrhetinic acid selected from the group consisting of hydrogen esters of glycyrrhetinic acid, salts of hydrogen esters of glycyrrhetinic acid, carbenoxolone, and combinations thereof; and a pharmaceutically acceptable derivative of quinine selected from the group consisting of quinidine, mefloquine, and combinations thereof.

In an aspect, the disclosure provides a method of treating acute myeloid leukemia in a subject in need thereof, the method comprising administering to the subject an effective amount of an agent or gap junction blocker, thereby treating acute myeloid leukemia in the subject.

In some embodiments, the agent or gap junction blocker comprises an inhibitor of 11β-hydroxysteroid dehydrogenase (11β-HSD). In some embodiments, the agent or gap junction blocker comprises carbenoxolone or an analog thereof. In some embodiments, the agent or gap junction blocker is not 18-β-glycyrrhetinic acid. In some embodiments, the agent or gap junction blocker selectively eradicates leukemic cells in the subject without eradicating normal leukocytes in the subject. In some embodiments, the agent or gap junction blocker selectively eradicates leukemic cells in the subject with minimal eradication of normal leukocytes in the subject. In some embodiments, the agent or gap junction blocker selectively eradicates leukemic cells in the subject while inducing proliferation of normal leukocytes in the subject.

In some embodiments, the method further includes administering an induction chemotherapy treatment regimen to the subject. In some embodiments, the induction chemotherapy comprises administering an antimetabolite agent and an anthracycline agent to the subject. In some embodiments, the antimetabolite agent comprises cytarabine. In some embodiments, the anthracycline agent comprises doxorubicin. In some embodiments, the induction chemotherapy comprises administering cytarabine and doxorubicin to the patient for a period of 5 days. In some embodiments, the induction chemotherapy comprises administering cytarabine and doxorubicin to the patient for a period of 3 days, followed by administering cytarabine alone to the patient for a period of 2 days.

In some embodiments, the agent or gap junction blocker is administered to the subject for at least a day before administering the induction chemotherapy treatment regimen to the subject. In some embodiments, the agent or gap junction blocker is administered to the subject for at least a day before administering the induction chemotherapy treatment regimen to the subject concomitantly with the agent or gap junction blocker.

In some embodiments, the subject is suffering from refractory or relapsed acute myeloid leukemia. In some embodiments, the method further includes evaluating the subject to determine if the subject has refractory or relapsed acute myeloid leukemia.

In some embodiments, the subject is a subject who relapses from complete remission of acute myeloid leukemia after induction chemotherapy.

In some embodiments, treating acute myeloid leukemia comprises inducing complete remission of acute myeloid leukemia in the subject.

In some embodiments, treating acute myeloid leukemia comprises inducing complete remission of acute myeloid leukemia in the subject in the absence of a relapse risk due to residual leukemic cells in the subject's bone marrow or peripheral blood.

In an aspect, the disclosure provides a method of promoting survival of a subject suffering from acute myeloid leukemia, the method comprising administering to the subject an effective amount of an agent or gap junction blocker, thereby promoting survival of the subject.

In some embodiments, the agent or gap junction blocker comprises an inhibitor of 11β-hydroxysteroid dehydrogenase (11β-HSD). In some embodiments, the agent or gap junction blocker comprises carbenoxolone or an analog thereof. In some embodiments, the method further includes administering an induction chemotherapy treatment regimen to the subject. In some embodiments, the induction chemotherapy comprises administering an antimetabolite agent and an anthracycline agent to the subject. In some embodiments, the antimetabolite agent comprises cytarabine. In some embodiments, the anthracycline agent comprises doxorubicin. In some embodiments, the induction chemotherapy comprises administering cytarabine and doxorubicin to the patient for a period of 5 days. In some embodiments, the induction chemotherapy comprises administering cytarabine and doxorubicin to the patient for a period of 3 days, followed by administering cytarabine alone to the patient for a period of 2 days.

In some embodiments, the agent or gap junction blocker is administered to the subject for at least a day before administering the induction chemotherapy treatment regimen to the subject. In some embodiments, the agent or gap junction blocker is administered to the subject for at least a day before administering the induction chemotherapy treatment regimen to the subject concomitantly with the agent or gap junction blocker.

In some embodiments, the method further includes selecting a subject suffering from or exhibiting a terminal state of acute myeloid leukemia. In some embodiments, the subject has advanced tumor metastasis. In some embodiments, the subject has a high tumor burden.

In some embodiments, the agent or gap junction blocker increases the subject's length of survival compared to the subject's length of survival in the absence of receiving the agent or gap junction blocker. In some embodiments, the agent or gap junction blocker increases the subject's likelihood of survival compared to the subject's likelihood of survival in the absence of receiving the agent or gap junction blocker.

In an aspect, the disclosure provides a method of inducing complete remission in a subject having relapsed or refractory acute myeloid leukemia by selectively eradicating leukemic cells in the subject, the method comprising: (a) evaluating the subject to determine if the subject has relapsed or refractory acute myeloid leukemia; (b) administering to the subject an agent or gap junction blocker at least a day before administering an induction chemotherapy treatment regimen to the subject; and (c) administering to the subject an induction chemotherapy treatment regimen comprising an antimetabolite agent and an anthracycline agent for proscribed periods of time, thereby inducing complete remission in the subject by selectively eradicating leukemic cells in the subject.

In an aspect, the disclosure provides a pharmaceutical composition comprising an effective amount of an agent or gap junction blocker, an effective amount of at least one chemotherapeutic agent subject to resistance by acute myeloid leukemia, and a pharmaceutically acceptable carrier, diluent, or excipient.

In some embodiments, the at least one chemotherapeutic agent subject to resistance by acute myeloid leukemia comprises an antimetabolite agent. In some embodiments, the at least one chemotherapeutic agent subject to resistance by acute myeloid leukemia comprises cytarabine. In some embodiments, the at least one chemotherapeutic agent subject to resistance by acute myeloid leukemia comprises an anthracycline agent. In some embodiments, the at least one chemotherapeutic agent subject to resistance by acute myeloid leukemia comprises doxorubicin. In some embodiments, the at least one chemotherapeutic agent subject to resistance by acute myeloid leukemia comprises an antimetabolite agent and anthracycline agent. In some embodiments, the at least one chemotherapeutic agent subject to resistance by acute myeloid leukemia comprises cytarabine and doxorubicin.

In some embodiments, the agent or gap junction blocker comprises carbenoxolone or an analog thereof. In some embodiments, the agent or gap junction blocker is not 18-β-glycyrrhetinic acid.

In an aspect, the disclosure provides a kit comprising an agent or gap junction blocker, at least one chemotherapeutic agent subject to resistance by acute myeloid leukemia, and instructions for administering the agent or gap junction blocker and the at least one chemotherapeutic agent to a subject suffering from acute myeloid leukemia.

In some embodiments, the kit further comprises a prophylactic treatment to be administered with the agent or gap junction blocker and/or the at least one chemotherapy agent, and instructions for administering the prophylactic treatment with the agent or gap junction blocker and/or the at least one chemotherapy agent. In some embodiments, the prophylactic treatment comprises a pharmaceutically active agent described herein for treating or preventing hypertension, hypokalemia, and edemas. In some embodiments, the instructions further comprise directions for administering the at least one chemotherapeutic agent as part of an induction chemotherapy treatment regimen for the subject. In some embodiments, the instructions further comprise directions for administering the agent or gap junction blocker, and the at least one therapeutic agent to induce complete remission of acute myeloid leukemia in the subject. In some embodiments, the instructions further comprise directions for administering the agent or gap junction blocker, and the at least one therapeutic agent to induce complete remission of acute myeloid leukemia in the subject, without risk of relapse by completely eradicating leukemic cells in the subject. In some embodiments, the instructions further comprise directions for administering the agent or gap junction blocker, and the at least one therapeutic agent to induce complete remission of acute myeloid leukemia in the subject by completely eradicating leukemic cells in the subject by inducing the leukemic cells to differentiate from proliferating, immortalized leukemic cells into short-lived, non-leukemic cells.

In some embodiments, the at least one chemotherapeutic agent subject to resistance by acute myeloid leukemia comprises an antimetabolite agent. In some embodiments, the at least one chemotherapeutic agent subject to resistance by acute myeloid leukemia comprises cytarabine. In some embodiments, the at least one chemotherapeutic agent subject to resistance by acute myeloid leukemia comprises an anthracycline agent. In some embodiments, the at least one chemotherapeutic agent subject to resistance by acute myeloid leukemia comprises doxorubicin. In some embodiments, the at least one chemotherapeutic agent subject to resistance by acute myeloid leukemia comprises an antimetabolite agent and an anthracycline agent. In some embodiments, the at least one chemotherapeutic agent subject to resistance by acute myeloid leukemia comprises cytarabine and the anthracycline agent comprises doxorubicin.

In some embodiments, the agent or gap junction blocker comprises carbenoxolone or an analog thereof.

The above discussed and many other features and attendant advantages of the present invention will become better understood by reference to the following detailed description of the invention when taken in conjunction with the accompanying examples.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A, 1B, 1C and 1D demonstrate the results of kinetic studies of a 5 day induction chemotherapy regimen administered in a mouse model of acute myeloid leukemia (AML). FIG. 1A is a schematic illustration shows the experimental design for the mouse model of AML. FIG. 1B is an example of IVIS imaging of mice at day 14, showing luciferase activity in the bones. FIGS. 1C and 1D are bar graphs showing the results of FACS analysis of GFP-positive MLL-AF9 cells from blood (FIG. 1C) and bone marrow (BM) (FIG. 1D) samples.

FIGS. 2A, 2B, 2C and 2D demonstrate that gap junctions activity plays a key role in maintaining leukemic cell drug resistance. 100,000 MLL-AF9 cells were incubated for 16-hours with or w/o chemotherapy (50 nM Cytarabine+20 nM Doxorubicin) and with or w/o sub-confluent MS-5 layer (FIG. 2A). 100,000 MLL-AF9 cells co-cultured with sub-confluent MS-5 layer, with or w/o transwell inserts (0.4 μm), were incubated for 16-hours with or w/o chemotherapy (50 nM Cytarabine+20 nM Doxorubicin) and with or w/o 100 μM CBX (FIGS. 2B and 2C). Cell viability was determined by % of 7AAD-negative cells in FACS analysis (*p<0.01). FIG. 2D shows whole body bioluminescence imaging (IVIS) 1 week after indicated treatment. White arrows indicate minimal residual AML cells.

FIGS. 3A and 3B demonstrate that in vivo administration of Carbenoxolone alone or in combination with chemotherapy, to mice transplanted with MLL-AF9 leukemia (established mouse model of human leukemia), results in prolonged survival. FIG. 3A shows survival curves of mice left untreated (red line), or mice that received treatment on Day 27, upon detection of Leukemic cells in the bones (green, blue, purple or black lines, as indicated). Chemo: cytarabine (100 mg/kg) and doxorubicin (3 mg/kg) to the subject for a period of 3 days, followed by administering cytarabine alone (100 mg/kg) to the subject for a period of 2 days. CBX: Carbenoxolone (20 mg/kg) to the subject for a period of 3 or 6 days, with or w/o chemotherapy. FIG. 3B shows survival curves of terminally ill mice that left untreated (red line), or that received treatment on Day 68 (blue line), a terminal state of the disease, with leukemic cells spread all over the body and high tumor burden. CBX: Carbenoxolone (10 mg/kg) to the subject for a period of 3 days, followed by 3 days without treatment, followed by 2 days of Carbenoxolone (20 mg/kg) to the subject, followed by 3 days without treatment, followed by 3 days of Carbenoxolone (30 mg/kg) to the subject.

FIGS. 4A, 4B, 4C and 4D demonstrate the selective eradication of different AML cell types by gap junction blockade in vitro. Carbenoxolone (CBX), at the indicated concentrations, was added to either 100,000 MLL-AF9, primary bone marrow leukocytes or primary peripheral blood leukocytes for 16 hours (FIG. 4A). Either 100,000 MLL-AF9 (clone A) cells (FIG. 4B), 100,000 MLL-AF9 (clone B) cells (FIG. 4C), or 100,000 HoxA9/Meis1 cells (FIG. 4D) were mixed with 100,000 primary BM leukocytes and exposed to CBX, as indicated, for 16 hours. Cells were distinguished by Cd45.1/CD45.2 expression and cell viability was determined by % of 7AAD-negative cells, in FACS analysis. (*p<0.01).

FIGS. 5A, 5B, 5C and 5D demonstrate that carbenoxolone selectively eradicates different murine AML cells (blue bars) without affecting non-leukemic normal counterparts (red bars). FIG. 5A is a bar graph showing the results of MLL-AF9 (clone A) cells mixed 1:1 with freshly isolated primary blood leukocytes and exposed to carbenoxolone, as indicated, for 16 hours. MLL-AF9 (clone A) cells (FIG. 5B), MLL-AF9 (clone B) cells (FIG. 5C), and HoxA9/Meis1 cells (FIG. 5D) were mixed at a 1:1 ratio with freshly isolated bone marrow leukocytes, and exposed to carbenoxolone, as indicated, for 16 hours. Cells were distinguished by CD45.1/CD45.2 expression and cell viability was determined by % of 7AAD-negative cells, in FACS analysis (*p<0.01).

FIGS. 6A, 6B, 6C, 6D, 6E, and 6F demonstrate that carbenoxolone treatment eradicates murine leukemic cancer stem cells while inducing proliferation of normal stem cells. Colony formation assays were performed on MLL-AF9 cells (FIG. 6A), bone marrow primary leukocytes (FIG. 6B), and a 1:1 mixture of both MLL-AF9 cells and bone marrow primary leukocytes (FIG. 6C) exposed to increasing concentrations of carbenoxolone, as indicated, and the colony forming units in culture CFU-C per 10,000 cells was determined at each concentration. FIGS. 6D, 6E and 6F are images showing formation of leukemic colonies (green) and normal colonies (blue) upon exposure to 0 μM (FIG. 6D), 50 μM (FIG. 6E), and 200 μM or 100 μM (FIG. 6F) of carbenoxolone.

FIGS. 7A, 7B, and 7C demonstrate that a gap-junction blockade promotes differentiation of AML cells into short-lived granulocytes in vitro. Primary MLL-AF9 cells were exposed to increasing concentrations of carbenoxolone, as indicated, for 16 hours and then analyzed by FACS analysis for the expression of Gr1 (FIG. 7A), Mac1 (FIG. 7B), or Gr1/Mac1 (FIG. 7C). Cell viability was determined by % of 7AAD-negative cells, in FACS analysis. (*p<0.01).

FIGS. 8A, 8B, 8C, 8D, 8E, 8F, 8G, 8H, 8I, 8J, 8K, and 8L demonstrate that carbenoxolone induces rapid (within 16 hours) neutrophil differentiation (CD66b+/CD14−) in human AML cell line U937. Human AML U937 cells were exposed to increasing concentrations of carbenoxolone, as indicated, for 16 hours and then analyzed by FACS analysis for the expression of live cells (FIG. 8A), CD14+ (FIG. 8B), CD66b+ (FIG. 8C), Mac1+ (FIG. 8D), CD66b+/Mac1− (FIG. 8E), CD66b+/Mac1+ (FIG. 8F), CD66b−/Mac1+ (FIG. 8G), CD66b−/Mac1− (FIG. 8H), CD66+/CD14− (FIG. 8I), CD66b+/CD14+ (FIG. 8J), CD66b−/CD14+ (FIG. 8K), and CD66b−/CD14− (FIG. 8L). Cell viability was determined by % of 7AAD-negative cells, in FACS analysis. (*p<0.01).

FIGS. 9A, 9B, 9C, 9D, 9E, 9F, 9G, 9H, 9I, 9J, 9K, and 9L demonstrate that carbenoxolone induces rapid (within 16 hours) neutrophil differentiation (CD66b+/CD14−) in human AML cell line HL60. Human AML HL60 cells were exposed to increasing concentrations of carbenoxolone, as indicated, for 16 hours and then analyzed by FACS analysis for the expression of live cells (FIG. 9A), CD14+ (FIG. 9B), CD66b+ (FIG. 9C), Mac1+ (FIG. 9D), CD66b+/Mac1− (FIG. 9E), CD66b+/Mac1+(FIG. 9F), CD66b−/Mac1+ (FIG. 9G), CD66b−/Mac1− (FIG. 9H), CD66+/CD14− (FIG. 9I), CD66b+/CD14+ (FIG. 9J), CD66b−/CD14+ (FIG. 9K), and CD66b−/CD14− (FIG. 9L). Cell viability was determined by % of 7AAD-negative cells, in FACS analysis. (*p<0.01).

FIGS. 10A, 10B, 10C, 10D, 10E, 10F, 10G, 10H, 10I, 10J, 10K and 10L demonstrate that carbenoxolone does not induce neutrophil differentiation (CD66b+/CD14−) in freshly isolated primary human leukocytes. Primary human leukocytes were freshly isolated and exposed to increasing concentrations of carbenoxolone, as indicated, for 16 hours and then analyzed by FACS analysis for the expression of live cells (FIG. 10A), CD14+ (FIG. 10B), CD66b+ (FIG. 10C), Mac1+(FIG. 10D), CD66b+/Mac1− (FIG. 10E), CD66b+/Mac1+ (FIG. 10F), CD66b−/Mac1+(FIG. 10G), CD66b−/Mac1− (FIG. 10H), CD66+/CD14− (FIG. 10I), CD66b+/CD14+(FIG. 1J), CD66b−/CD4−+ (FIG. 10K), and CD66b−/CD14− (FIG. 10L). Cell viability was determined by % of 7AAD-negative cells, in FACS analysis. (*p<0.01).

FIGS. 11A, 11B, 11C and 11D demonstrate that Carbenoxolone treatment eradicates human leukemic cancer stem and progenitor cells (11A-11C) while inducing proliferation of normal human stem and progenitor cells (11D). Colony formation assays were performed on THP1 cells (FIG. 11A), HL60 (FIG. 11B), U937 (FIG. 11C) and freshly isolated primary non-leukemic normal human leukocytes (FIG. 11D) exposed to increasing concentrations of Carbenoxolone, as indicated, and the colony forming units in culture CFU-C per 2,000 cells was determined at each concentration.

FIGS. 12A, 12B, 12C, 12D, 12E, 12F, 12G, 12H, 12I, 12J, 12K, 12L, 12M, 12N, 12O, 12P, and 12Q are gene expression profiles from published arrays demonstrating that 11βHSD, and members of the Connexin gap junction family are over-expressed in human AML. FIG. 12A is an expression profile showing 11βHSD expression in malignant and normal blood. FIG. 12B is an expression profile showing Cx40.1 expression in malignant and normal blood. FIG. 12C is an expression profile showing Cx30.2 expression in malignant and normal blood. FIG. 12D is an expression profile showing Cx31.1 expression in malignant and normal blood. FIG. 12E is an expression profile showing Cx36 expression in malignant and normal blood. FIG. 12F is an expression profile showing Cx45 expression in malignant and normal blood. FIG. 12G is an expression profile showing Cx47 expression in malignant and normal blood. FIG. 12H is an expression profile showing Cx32 expression in malignant and normal blood. FIG. 12I is an expression profile showing Cx50 expression in malignant and normal blood. FIG. 12J is an expression profile showing Cx30.3 expression in malignant and normal blood. FIG. 12K is an expression profile showing Cx31 expression in malignant and normal blood. FIG. 12L is an expression profile showing Cx26 expression in malignant and normal blood. FIG. 12M is an expression profile showing Cx40 expression in malignant and normal blood. FIG. 12N is an expression profile showing Cx37 expression in malignant and normal blood. FIG. 12O is an expression profile showing Cx46 expression in malignant and normal blood. FIG. 12P is an expression profile showing Cx43 expression in malignant and normal blood. FIG. 12Q is an expression profile showing Cx30 expression in malignant and normal blood. Every dot in each of the expression profiles depicted in FIGS. 12A-12Q represents a different independent study.

FIG. 13 demonstrates an experimental design for the real time analysis of leukemic cell interactions and communication during induction chemotherapy, which enables intravital microscopy, monitoring of disease progression, synchronization of treatment, monitoring of relapse, valid comparison of different oncogenes, minimization of use of viruses, in vitro live video microscopy, and in vitro screens.

FIGS. 14A, 14B and 14C demonstrate that CBX overcomes stroma-mediated drug resistance. Results obtained using 25 nM cytarabine and 10 nM doxorubicin as chemo. Asterisks indicate results obtained after 16 hours of incubation. Viability was detected via GFP expression and 7AAD exclusion. Results in FIG. 14C achieved with transwell insert separation.

FIG. 15 demonstrates that in vitro exposure to CBX (at greater than or equal to 100 μM) leads to the eradication of MLL-AF9 leukemia, but not the eradication of normal blood cells. Results obtained via flow cytometry viability assays after 16 hours of incubation with the amounts of CBX indicated with viability detection by GFP expression and 7AAD exclusion.

FIGS. 16A, 16B, 16C and 16D demonstrate that CBX selectively eradicates different murine AML cells (blue bars) without affecting non-leukemic normal counterparts (red bars)—in 1:1 cell mixtures. FIG. 16A shows results of CBX treatment at the concentrations indicated of MLL-AF9 (bulk A) cells mixed 1:1 with freshly isolated blood leukocytes. FIG. 16B shows results of CBX treatment at the concentrations indicated of MLL-AF9 (bulk A) cells mixed 1:1 with freshly isolated bone marrow leukocytes. FIG. 16C shows results of CBX treatment at the concentrations indicated of MLL-AF9 (bulk B) cells mixed 1:1 with freshly isolated bone marrow leukocytes. FIG. 16D shows results of CBX treatment at the concentrations indicated of HoxA9/Meis1 cells mixed 1:1 with freshly isolated bone marrow leukocytes.

FIGS. 17A, 17B, 17C, and 17D demonstrates the differential effect of CBX (at greater than or equal to 50 μM) on malignant vs. normal immature stem and progenitor cells (CFU-C assay). FIG. 17A is a bar graph showing results of CBX treatment at the concentrations indicated on MLL-AF9 leukemic cells alone. FIG. 17B is a bar graph showing results of CBX treatment at the concentrations indicated on normal bone marrow primary leukocytes alone. FIG. 17C is a bar graph showing results of CBX treatment at the concentrations indicated on a 1:1 mixture of MLL-AF9 cells and normal bone marrow primary leukocytes. FIG. 17D shows images of the results of CBX treatment on the cell mixture described in FIG. 17C.

FIGS. 18A, 18B, 18C and 18D demonstrate that the human APL cell line HL60 is sensitive to CBX. FIG. 18A is a bar graph showing the viability of HL60 cells 16 hours post exposure to CBX at the concentrations indicated, as assessed by flow cytometry. FIG. 18B is a bar graph showing the viability of freshly isolated human peripheral blood leukocytes 16 hours post exposure to CBX at the concentrations indicated, as assessed by flow cytometry. FIG. 18C is a bar graph showing colony formation of HL60 cells 7 days post exposure to CBX at the concentrations indicated, as assessed by CFU-C assay for a functional quantification of cancer and normal stem and progenitor cells. FIG. 18D is a bar graph showing colony formation of freshly isolated human peripheral blood leukocytes 7 days post exposure to CBX at the concentrations indicated, as assessed by CFU-C assay for a functional quantification of cancer and normal stem and progenitor cells.

FIGS. 19A, 19B, 19C and 19D demonstrate that CBX treatment (100 μM) eradicates human leukemic progenitors but induces proliferation of normal progenitors. FIG. 19A is a bar graph showing proliferation of THP1 cells treated with CBX at the concentrations indicated, as assessed by CFU-C assay. FIG. 19B is a bar graph showing proliferation of HL60 cells treated with CBX at the concentrations indicated, as assessed by CFU-C assay. FIG. 19C is a bar graph showing proliferation of U937 cells treated with CBX at the concentrations indicated, as assessed by CFU-C assay. FIG. 19D is a bar graph showing proliferation of primary human progenitor cells treated with CBX at the concentrations indicated, as assessed by CFU-C assay.

FIGS. 20A, 20B and 20C demonstrate that CBX rapidly induces apoptosis of MLL-AF9 leukemic cells. Blue bars and red bars show viability (as assessed by the frequency of 7AAD-negative cells) and apoptosis (as assessed by frequency of 7AAD-negative, annexin-V positive cells) of MLL-AF9 leukemic cells after treatment with CBX at the concentrations indicated compared to control for 6 hours (FIG. 20A), 12 hours (FIG. 20B), and 20 hours (FIG. 20C).

FIG. 21 demonstrates that CBX treatment induces apoptosis of leukemic cells and enhances the replenishment of normal healthy cells. FACS dot plots show the results of CBX treatment compared to control (PBS) after 4h on a 1:1 mixture of iRFP+ MLL-AF9 leukemic cells with normal bone marrow mononuclear cells (BM-MNC).

FIG. 22 demonstrates that CBX treatment induces apoptosis of leukemic cells and enhances the replenishment of normal healthy cells. Blue bars and red bars show apoptotic mononuclear cells (MNC) and apoptotic leukemic cells, respectively, after treatment with CBX at the concentrations indicated compared to the PBS control.

FIG. 23 demonstrates that MLL-AF9 leukemic cells are double-positive for Mac1, Gr1 and cKit. FACS dot plots indicate that normal myoblasts are the most appropriate counterparts for comparison.

FIG. 24 shows FACTS dot plots showing the results of CBX treatment compared to control (PBS) after 4 h on a 1:1 mixture of iRFP+ MLL-AF9 leukemic cells with normal myeloblasts.

FIG. 25 demonstrates that CBX-induced leukemia apoptosis is not cell cycle dependent. FIG. 25 is a bar graph showing results of 4 h of CBX treatment on a 1:1 mixture of iRFP+ MLL−AF9 leukemic cells (blue bars) with highly proliferating normal myeloblasts (black bars) or expanded lineage−/cKit+/Sca1+ (LKS) cells (black bars). Rate of proliferation for cell populations used is as follows: MLL-AF9: 3 divisions/24 hours; myeloblasts: 3 divisions/24 hours, eLKS: 7 divisions/24 hours.

FIGS. 26A, 26B, 26C and 26D. FIGS. 26A and 26B are bar graphs showing the viability (7AAD-negative, iRFP+/−; FIG. 26A) and apoptosis (7AAD-negative, iRFP+, annexin-V-positive; FIG. 26B) of a 1:1 mixture of MLL-AF9 iRFP+ leukemia and normal myeloblast cells after 4 h treatment with 200 μM of CBX or Aldosterone. FIGS. 26C and 26D are bar graphs showing the viability (7AAD-negative, iRFP+/−; FIG. 26C) and apoptosis (7AAD-negative, iRFP+, annexin-V-positive; FIG. 26D) of a 1:1 mixture of MLL-AF9 iRFP+ leukemia and normal myeloblast cells after 24 h treatment with 200 μM of CBX or Aldosterone.

FIGS. 27A, 27B, 27C and 27D. FIGS. 27A and 27B are bar graphs showing the viability (7AAD-negative, GFP+/−; FIG. 27A) and apoptosis (7AAD-negative, GFP+/−, annexin-V-positive; FIG. 27B) of a 1:1 mixture of MLL-AF9 GFP+ leukemia and normal myeloblast cells after 4 h treatment with 200 μM of CBX or Aldosterone. FIGS. 27C and 27D are bar graphs showing the viability (7AAD-negative, GFP+/−; FIG. 26C) and apoptosis (7AAD-negative, GFP+/, annexin-V-positive; FIG. 27D) of a 1:1 mixture of MLL-AF9 GFP+ leukemia and normal myeloblast cells after 24 h treatment with 200 μM of CBX or Aldosterone.

FIGS. 28A-N depict compounds with similar structure and/or similar systemic “steroid-like” effects, to CBX (FIG. 28N). FIGS. 28A, 28B, 28C, and 28D are the structural formulas for mineralocorticoids aldosterone (FIG. 28A), spironolactone (FIG. 28B), fludrocortisone (FIG. 28C), and deoxycorticosterone (FIG. 28D). FIGS. 28E, 28F, 28G, 28H, 28I, 28J, 28K 28L and 28M are the structural formulas for glucocorticoids beclometasone dipropionate (FIG. 28E), cortisol (FIG. 28F), cortisone (FIG. 28G), dexamethasone (FIG. 28H), betamethasone (FIG. 28I), prednisolone (FIG. 28J), prednisone (FIG. 28K), methylprednisolone (FIG. 28L), and triamcinolone acetonide (FIG. 28M).

FIGS. 29A and 29B. FIG. 29A is a bar graph showing the viability (7AAD-negative, iRFP+/−) of a 1:1 mixture of MLL-AF9 iRFP+ leukemia and normal myeloblast cells after 24 h treatment with 200 μM of CBX or the mineralocorticoid compounds indicated. FIG. 29B is a Table showing the comparative steroid potencies of the compounds shown in FIGS. 28A-28N.

FIGS. 30A and 30B. FIG. 30A is a bar graph showing the viability (7AAD-negative, iRFP+/−) of a 1:1 mixture of MLL-AF9 iRFP+ leukemia and normal myeloblast cells after 24 h treatment with 200 μM of CBX or the glucocorticoid compounds indicated. FIG. 30B is a Table showing the comparative steroid potencies of the compounds shown in FIGS. 28A-28N.

FIGS. 31A and 31B. FIG. 31A is a bar graph showing the viability (7AAD-negative, iRFP+/−) of a 1:1 mixture of MLL-AF9 iRFP+ leukemia and normal myeloblast cells after 24 h treatment with 200 μM of CBX or the glucocorticoid compounds indicated. FIG. 31B is a Table showing the comparative steroid potencies of the compounds shown in FIGS. 28A-28N.

FIG. 32 demonstrates that Mac1 (CD11b, integrin αₘ) is downregulated in CBX treated mice leukemic cells undergoing apoptosis (7AAD-negative MLL-AF9 cells).

FIG. 33 is a Table demonstrating that human myelo-markers are different than the mouse. Mac1 is expressed by neutrophils, NK cells and macrophages. CD66b is expressed exclusively on granulocytes and used as a granulocyte marker. CD14 is expressed mainly by macrophages (and at 10-times lesser extend by neutrophils). CD66b+ CD14+ marks only monocytes. As shown in FIG. 33, CD14 is the human equivalent to Mac-1 in mice, and CD66b is the human equivalent to Gr-1 in mice.

FIGS. 34A, 34B, 34C and 34D demonstrate that Mac1 is downregulated in CBX-treated human leukemias, but not in CBX-treated normal human blood cells, as determined by flow cytometry analysis of live 7AAD human cells incubated with CBX at the concentrations indicated for 16 h. FIGS. 34A-34D are bar graphs quantifying myelo-markers CD11b (FIG. 34A), CD14+ (FIG. 34B), CD66b+ (FIG. 34C), and CD66+/CD14+ (FIG. 34D) in CBX-treated primary normal human leukocytes as compared to CBX-treated human leukemic cell lines U937, HL60 and THP1.

FIG. 35 is a Table demonstrating results of an in vivo gene expression study of gap-junction molecules in bone marrow leukemic cells and their normal counterparts. Bone marrow leukemic cells assessed comprise MLL-AF9+, GFP-positive, Gr1/Mac1-positive, c-Kit high cells. Bone marrow normal myeloid progenitors assessed comprise B220/CD8a/CD3e/CD4/TERI 19-negative GFP-negative, GR1/Mac-1-positive, c-Kit high cells.

FIG. 36 is a bar graph illustrating expression of connexin sorting protein Consortin relative to expression of HPRT in normal GMP compared to leukemia after using the treatments and controls indicated for the time periods specified.

FIGS. 37A, 37B, 37C, 37D, 37E, 37F, 37G, 37H, and 371 are bar graphs illustrating expression of gap-junction alpha molecules relative to expression of HPRT in normal GMP compared to leukemia after using the treatments and controls indicated for the time periods specified, including gap-junction alpha molecules A1 (FIG. 37A), A3 V1 (FIG. 37B), A3 V2 (FIG. 37C), A4 (FIG. 37D), A5 V1 (FIG. 37E), A5 V2 (FIG. 37F), A6 (FIG. 37G), A8 (FIG. 37H) and A10 (FIG. 37I).

FIGS. 38A, 38B, 38C, 38D, 38E, 38F and 38G are bar graphs illustrating expression of gap-junction beta molecules relative to expression of HPRT in normal GMP compared to leukemia after using the treatments and controls indicated for the time periods specified, including gap-junction beta molecules B1 (FIG. 38A), B2 (FIG. 38B), B3 (FIG. 38C), B4 (FIG. 38D), B5 (FIG. 38E), B6 V3 (FIG. 38F) and B6 V2 (FIG. 38G).

FIGS. 39A, 39B and 39C are bar graphs illustrating expression of gap-junction gamma molecules relative to expression of HPRT in normal GMP compared to leukemia using the treatments and controls indicated for the time periods specified, including gap-junction gamma molecules C1 (FIG. 39A), C2 (FIG. 39B) and C3 (FIG. 39C).

FIGS. 40A, 40B and 40C are bar graphs illustrating expression of gap-junction delta molecules relative to expression of HPRT in normal GMP compared to leukemia using the treatments and controls indicated for the time periods specified, including gap-junction delta molecules D2 (FIG. 40A), D3 (FIG. 40B) and D4 (FIG. 40C).

FIG. 41 is a bar graph illustrating expression of gap-junction epsilon molecules relative to expression of HPRT in normal GMP compared to leukemia after using the treatments and controls indicated for the time periods specified.

FIG. 42 demonstrates that intraperitoneal (IP) administration of CBX to MLL-AF9 leukemic mice results in prolonged survival. Leukemia was induced in mice by intravenously injecting 1 million live MLL-AF9 leukemic cells (expressing GFP and luciferase) into non-irradiated recipients. In the model, leukemic cells could be detected in the bone marrow ˜27 days post-transplantation and non-treated mice succumbed to leukemia at day ˜70. The mice were administered CBX intraperitoneally at 50 mg/kg 6 days beginning at day 27 upon detection of leukemia in the bone marrow. FIG. 42 shows whole body bioluminescence signal (IVIS) on day 36 of mice not treated (left), treated with chemotherapy (middle), and treated with CBX plus chemotherapy (right).

FIGS. 43A and 43B demonstrate that intraperitoneal (IP) administration of CBX to MLL-AF9 leukemic mice results in prolonged survival. For the experimental results depicted in FIG. 43A, treatment was given on day 27 upon detection of leukemic cells in the bones, chemo comprised cytarabine (100 mg/kg) and doxorubicin (3 mg/kg) for 3 days, followed by administering cytarabine alone (100 mg/kg) for an additional 2 days, and CBX was administered at 20 mg/kg to the subject for 6 days. For the experimental results depicted in FIG. 43B, treatment was given on day 68, at a terminal state of the disease, with leukemic cells spread all over the body, CBX was administered at 10 mg/kg for 3 days, followed by 3 days without treatment, followed by 2 days of 20 mg/kg, followed by 3 days without treatment, followed by 3 days of 30 mg/kg.

FIG. 44 demonstrates that daily sub-cutaneous (SC) administration of CBX for 2 weeks (at 75 mg/kg alone or at 50 mg/kg combined with chemotherapy) results in prolonged survival of leukemic mice. Leukemia was induced in mice by intravenously injecting 5 million live MLL-AF9 leukemic cells (expressing infrared fluorescent protein (iRFP) into sub-lethally irradiated recipients. In the model, leukemic cells could be detected in the bone marrow ˜7 days post-transplantation and non-treated mice succumbed to leukemia at day ˜34. The following treatment regiment was employed: Day −1: sub-lethal irradiation (4.5 Gy); Day 1: tail vein injection of 5M live iRFP+ MLL-AF9 leukemic cells; Days 7+8: PBS or CBX treatment (subcutaneous, as depicted; Days 9-11: PBS or CBX treatment+chemotherapy (100 mg/kg Cytarabine+3 mg/kg Doxorubicin) Days 12+13: PBS or CBX treatment+chemotherapy (100 mg/kg Cytarabine); Days 14-21: PBS or CBX treatment. As shown in FIG. 44, chronic systemic exposure of CBX (continuously for 2 weeks), without prophylactic treatment, resulted in acute lethal pseudo-hyperaldosteronism in some case (e.g., hypertension and gastric edemas)

FIGS. 45A, 45B and 45C demonstrate the survival curve of mice treated with 25 mg/kg CBX alone, or in combination with induction chemotherapy. 40%-60% of CBX-treated mice survived, with early mortality due to acute lethal pseudo-hyperadlosteronism. Notably, there was no statistically significant difference in survival of mice treated with CBX in combination with induction chemotherapy compared to both PBS and chemotherapy controls.

FIGS. 46A, 46B and 46C demonstrate the survival curve of mice treated with 50 mg/kg CBX alone, or in combination with induction chemotherapy. 50 mg/kg CBX alone resulted in early mortality with no statistically significant difference (20% of CBX-treated mice survived). However, in combination with chemotherapy, 50 mg/kg of CBX led to significant increased survival compared to both PBS and chemotherapy controls.

FIGS. 47A, 47B and 47C demonstrate the survival curve of mice treated with 75 mg/kg CBX alone, or in combination with induction chemotherapy. 75 mg/kg alone led to significant increased survival compared to PBS controls. However, in combination with chemotherapy, 75 mg/kg resulted in early mortality with no statistically significant difference (20% of CBX-treated mice survived).

FIG. 48 demonstrates survival curves and p values of all SC prolonged administration trials described in FIGS. 45A-C, FIGS. 46A-C and FIGS. 47A-C.

FIGS. 49A and 49B demonstrate that prolonged administration of CBX (SC, daily, 14 days) might result in lethal pseudo-hyperadlosteronism with no sign of leukemia. FIG. 49A is an autopsy image of a healthy control mice. FIG. 49B is an autopsy image of a mouse that died at day 28 after 50 mg/ml CBX treatment (FIG. 49B), which exhibited gastric edema.

FIGS. 50A, 50B and 50C demonstrate that early mortality in CBX treated animals (SC, daily for 14 das) is most likely induced by acute pseudo-hyperaldosteronism, and not by leukemia. FIG. 50A is an autopsy image of a mouse treated with PBS, showing clear signs of leukemia. FIG. 50B is an autopsy image of a mouse treated with chemotherapy, showing signs of leukemia. FIG. 50C is an autopsy image of a mouse treated with chemotherapy plus 50 mg/kg CBX, showing no signs of leukemia. Black arrows indicate swollen lymph nodes. Red arrows indicate splenomegaly. Blue arrows indicate gastric edema.

FIG. 51 is an autopsy image of a subject mouse treated with chemotherapy, showing signs of leukemia. Black arrows indicate swollen lymph nodes. Red arrows indicate splenomegaly. Blue arrows indicate gastric edema.

FIG. 52 is an autopsy image of a subject mouse treated with chemotherapy plus 50 mg/kg CBX, showing no signs of leukemia. Black arrows indicate swollen lymph nodes. Red arrows indicate splenomegaly. Blue arrows indicate gastric edema.

FIG. 53 demonstrates the selective leukemia cell eradication by 18β-glycyrrhetinic acid (enoxolone) and depicts the structure thereof. Primary MLL-AF9^{LKS} acute myeloid leukemia cells (red bars) were co-cultured with normal BM mononuclear cells (blue bars). Cells in co-culture were exposed to enoxolone for 20 hours (at 0, 5, 20, 50, 100 and 200 μM) and then cell viability was analyzed by flow cytometry (live leukemia cells=GFP+/7AAD−; live normal cells=GFP−/7AAD−).

FIG. 54 shows the effects of 18β-glycyrrhetinic acid (enoxolone) on the viability of normal bone marrow cells (myeloid cells vs. non-myeloid cells). Cells in co-culture were exposed to enoxolone for 20 hours (at 0, 5, 20, 50, 100 and 200 μM). As illustrated in FIG. 54, enoxolone was toxic to normal non-myeloid cells at 200 μM.

### DETAILED DESCRIPTION OF THE INVENTION

The disclosure relates to the discovery that certain agents (e.g., glycyrrhetinic acid derivatives) can be used effectively to eradicate leukemic cells in a population or subject without eradicating normal cells in the subject or, in certain instances only minimally eradicating normal cells in the subject. Accordingly, the disclosure contemplates the use of one or more agents (e.g., glycyrrhetinic acid derivatives) in methods, compositions, and kits for eradicating leukemic cells, and in related methods, compositions, and kits for treating acute myeloid leukemia.

Eradicating Leukemic Cells

In some aspects, disclosed herein are methods for eradicating leukemic cells in a population of cells. Such methods are useful for, amongst other things, treating leukemia (e.g., acute myeloid leukemia). In one embodiment, a method of eradicating leukemic cells in a population of cells comprises contacting the population of cells with an effective amount of a gap junction blocker, thereby eradicating leukemic cells in the cell population.

It should be appreciated that the leukemic cells can be eradicated by a variety of mechanisms upon exposure to or contact with a gap junction blocker described herein. In some embodiments, eradicating leukemic cells comprises inducing programmed cell death. In some embodiments, eradicating leukemic cells comprises inducing apoptosis. In some embodiments, eradicating leukemic cells comprises inducing the differentiation of the leukemic cells into non-leukemic cells as described herein. In some embodiments, eradicating leukemic cells comprises inducing the differentiation of the leukemic cells into granulocytes. In some embodiments, the granulocytes comprise neutrophils. In some embodiments, leukemic cells are eradicated upon contact with a gap junction blocker described herein by inducing the differentiation of the leukemic cells (e.g., human) into CD66b+/CD14− neutrophils.

In other embodiments, eradicating leukemic cells comprises disrupting intercellular communications involving the leukemic cells that promote leukemia cell survival. In certain embodiments, eradicating leukemic cells comprises disrupting intercellular communications involving the leukemic cells that confer drug resistance to the leukemic cells. In some embodiments, disrupting intercellular communications involving leukemic cells comprises interfering with homotypic interactions between leukemic cells. In some embodiments, disrupting intercellular communications involving leukemic cells comprises interfering with heterotypic interactions between leukemic cells and stromal cells. In some embodiments, disrupting intercellular communications involving leukemic cells comprises interfering with heterotypic interactions between leukemic cells and any other cell types (e.g., mesenchymal stromal cells, osteolineage cells, endothelial cells, pericytes, mesenchymal cells or other hematopoietic cells). In certain embodiments, eradicating leukemic cells comprises overcoming stroma-mediated drug resistance (e.g., to cancer treatment, e.g., induction chemotherapy).

In some instances, disrupting intercellular communications involving the leukemic cells causes the leukemic cells to differentiate into non-leukemic cells, thereby eradicating the cells.

In some embodiments, leukemic cells are selectively eradicated while inducing proliferation of normal leukocytes in the population of cells. For example, contacting a population of acute myeloid leukemia cells with a gap junction blocker described herein selectively eradicates the acute myeloid leukemia cells while inducing the proliferation of normal leukocytes in the population.

It should be appreciated by those skilled in the art that the compositions and methods described herein preferably selectively affect leukemic cells without affecting normal cells (e.g., leukocytes) in the population of cells. In some embodiments, leukemic cells are selectively eradicated without eradicating, or in certain aspects minimally eradicating, normal leukocytes in the population of cells. For example, the leukemic cells are selectively eradicated without eradicating, or in certain embodiments minimally eradicating, normal bone marrow leukocytes or normal peripheral blood leukocytes, including without limitation, stem and progenitors, bone marrow mononuclear cells, myeloblasts, neutrophils, NK cells, macrophages, granulocytes, monocytes, and lineage−/cKit+/Sca1+ (LKS) cells. In some embodiments, the amount or activity of leukemic cells in a population of cells is selectively decreased without decreasing the amount or activity of normal leukocytes in the population. In some embodiments, proliferation of leukemic cells is selectively inhibited in a population of cells without inhibiting proliferation of normal leukocytes in the population. In some embodiments, the compositions and methods described herein can be used to increase the number of normal leukocytes in a population of cells by selectively reducing the number, activity, and/or proliferation of leukemic cells in the population of cells. Without wishing to be bound by theory, it is expected that the amount of leukemic cells eradicated, reduced, or inhibited in any particular population of cells is proportional to the concentration of gap junction blocker to which the population of cells has been exposed. In some instances, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or as much as 100% of the leukemic cells in the population of cells are eradicated, reduced, or inhibited by exposure to or contact with a gap junction blocker. In some embodiments, at least 20% of the leukemic cells in the population of cells are eradicated, reduced, or inhibited. In some embodiments, at least 50% of the leukemic cells in the population of cells are eradicated, reduced, or inhibited. In some embodiments, at least 70% of the leukemic cells in the population of cells are eradicated, reduced, or inhibited. In some embodiments, all of the leukemic cells in the population of cells are eradicated, reduced, or inhibited.

The present invention contemplates selectively eradicating any leukemic cell by contacting a population of cells with, or exposing the population of cells to, a gap junction blocker. In some embodiments, the leukemic cells comprise leukemia cells from an acute myeloid leukemia cell line. Exemplary acute myeloid leukemia cell lines include, but are not limited to, MLL-AF9 cells, MLL-ENL cells, Nup98-HoxA9 cells, AML1-ET09A cells, KG-1 cells, KG-1a cells, U937 cells, THP1 cells, HL60 cells, HoxA9/Meis1 cells, and NB-4 cells.

It should be appreciated that selective eradication of leukemic cells in a population of cells means that the leukemic cells in the population are eradicated without eradicating or otherwise affecting other normal cells in the population. In some embodiments, selective eradication of leukemic cells in a population of cells means that the leukemic cells in the population are eradicated with minimal eradication or with limited untoward effects on other normal cells within the population. In some embodiments, the population of cells comprises primary leukocytes, such as bone marrow leukocytes and peripheral blood leukocytes. Examples of such primary leukocytes include, without limitation, stem and progenitors, mononuclear cells, myeloblasts, neutrophils, NK cells, macrophages, granulocytes, monocytes, and lineage−/cKit+/Sca1+(LKS) cells.

It should be appreciated that the effective amount of the agents for use in accordance with the present inventions (e.g., a glycyrrhetinic acid derivative or a gap junction blocker) may vary, for example, depending on the agent or gap junction blocker being used and its location of use. In some embodiments, the effective amount of the agent (e.g., a glycyrrhetinic acid derivative or a gap junction blocker) for in vitro use comprises a concentration in the range of 50 μM to 400 μM. In some embodiments, the effective amount of the agent or gap junction blocker for in vivo use comprises a concentration in the range of 10 mg/kg to 100 mg/kg. In some embodiments, the effective amount comprises a concentration of 25 mg/kg. In some embodiments, the effective amount comprises a concentration of 50 mg/kg. In some embodiments, the effective amount comprises a concentration of 75 mg/kg.

In some embodiments, the contacting occurs in vitro or ex vivo.

In some embodiments, the contacting occurs in vivo. In some embodiments, the in vivo contact is in a subject as described herein.

Promoting Differentiation of Leukemic Cells into Non-Leukemic Cells

The disclosure provides methods for promoting the differentiation of leukemic cells into non-leukemic cells. Such methods can be useful for treating leukemia, for example, acute myeloid leukemia.

In one aspect, a method of promoting the differentiation of a leukemic cell into a non-leukemic cell comprises contacting a leukemic cell with an effective amount of an agent (e.g., a gap junction blocker), thereby promoting the differentiation of the leukemic cell into a non-leukemic cell.

The disclosure contemplates differentiating any leukemic cell into a non-leukemic cell in accordance with the methods described herein. In some embodiments, the leukemic cell comprises a leukemic stem or progenitor cell. In some embodiments, the leukemic stem or progenitor cell comprises an acute myeloid leukemia cell. In some embodiments, the acute myeloid leukemia comprises a cell line selected from the group consisting of MLL-AF9 cells, MLL-ENL cells, Nup98-HoxA9 cells, AML1-ET09A cells, KG-1 cells, KG-1a cells, U937 cells, HL60 cells, THP1 cells, HoxA9/Meis1 cells, and NB-4 cells.

It should be appreciated that the differentiated leukemic cells may differentiate into non-leukemic cells of varying stages. In some embodiments, the non-leukemic cell comprises a mature or terminally differentiated cell. In some embodiments, the non-leukemic cell comprises a granulocyte. In some embodiments, the granulocyte comprises a short-lived granulocyte. In some embodiments, the non-leukemic cell comprises a neutrophil. In some embodiments, the neutrophil comprises a CD66b+/CD14− neutrophil.

Methods of Treatment

The disclosure contemplates various methods of treatment utilizing the compositions and kits comprising the gap junction blockers and/or agents described herein. The disclosure contemplates the treatment of any disease in which intercellular communication or interactions through gap junctions or hemichannels plays a role in promoting survival of malignant or neoplastic cells or in which intercellular communication or interactions through gap junctions or hemichannels plays a role in disease resistance to treatment or therapy. The agents and/or gap junction blockers described herein can be used to treat and/or prevent such diseases. In some embodiments, the agents and/or gap junction blockers selectively eradicate malignant or neoplastic cells (e.g., blood cells) in which intercellular communication or interaction through gap junctions or hemichannels plays a role in disease resistance to treatment or therapy.

In some aspects, the disclosure provides a method of treating acute myeloid leukemia in a subject in need thereof, the method comprising administering to the subject an effective amount of a gap junction blocker or an agent (e.g., a glycyrrhetinic acid derivative) described herein, thereby treating acute myeloid leukemia in the subject.

In some embodiments, the agent and/or gap junction blocker selectively eradicates leukemic cells without eradicating, or minimally eradicating, normal cells in the subject. In some embodiments, the agent and/or gap junction blocker selectively eradicates leukemic cells in the subject without eradicating, or minimally eradicating, normal leukocytes in the subject. In some embodiments, the agent and/or gap junction blocker selectively eradicates leukemic cells in the subject while inducing proliferation of normal leukocytes in the subject. In some embodiments, the agent and/or gap junction blocker selectively eradicates leukemic cells in the subject while inducing replenishment of normal leukocytes in the subject. Of course, the agent and/or gap junction blocker can selectively eradicate leukemic cells without eradicating, or minimally eradicating, normal leukocytes while inducing the proliferation and/or replenishment of normal leukocytes in the subject.

In some embodiments, the method further comprises administering an induction chemotherapy treatment regimen to the subject. The disclosure contemplates administering any induction chemotherapy treatment regimen that is useful for inducing complete remission of acute myeloid leukemia in a subject. In some embodiments, the induction chemotherapy comprises administering an antimetabolite agent (e.g., cytarabine) and an anthracycline agent (e.g., doxorubicin) to the subject. In some embodiments, the antimetabolite agent comprises cytarabine. The induction chemotherapy treatment regimen can be administered to the subject over a period of hours, days, or months. The chemotherapeutic agents used in the induction chemotherapy treatment regimen can be administered at the same time throughout the period, or administered at different intervals within the period. In some embodiments, the induction chemotherapy comprises administering cytarabine and doxorubicin to the subject for a period of 5 days. In some embodiments, the induction chemotherapy comprises administering cytarabine and doxorubicin to the subject for a period of 3 days, followed by administering cytarabine alone to the subject for a period of 2 days.

The agent and/or gap junction blocker can be administered to the subject before the induction chemotherapy treatment regimen is administered to the subject, at the same time the induction chemotherapy treatment regimen is administered to the subject, after the induction chemotherapy treatment regimen is administered to the subject, or any combination of the above. In some embodiments, the agent and/or gap junction blocker is administered to the subject for at least a day before administering the induction chemotherapy treatment regimen to the subject. In some embodiments, the agent and/or gap junction blocker is administered to the subject for at least a day before administering the induction chemotherapy treatment regimen to the subject concomitantly with the agent and/or gap junction blocker. In some embodiments, the agent and/or gap junction blocker is administered to the subject at least 2 days, at least 3 days, at least 4 days, at least 5 days, at least 6 days, or up to at least a week before administering the induction chemotherapy treatment regimen to the subject. In some embodiments, the agent and/or gap junction blocker is administered to the subject at least 8 days, at least 9 days, at least 10 days, at least 11 days, at least 12 days, at least 13 days, at least 2 weeks, at least 3 weeks, or at least a month before the induction chemotherapy treatment regimen is administered to the subject. In some embodiments, the agent and/or gap junction blocker is administered to the subject for at least 1 day, at least 2 days, at least 3 days, at least 4 days, at least 5 days, at least 6 days, or up to at least a week before administering the induction chemotherapy treatment regimen to the subject, and then the induction chemotherapy regimen is administered to the subject concomitantly with the gap junction blocker for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 2 weeks, at least 3 weeks, or at least a month. In some embodiments, the agent and/or gap junction blocker is administered to the subject for at least 1 day, at least 2 days, at least 3 days, at least 4 days, at least 5 days, at least 6 days, or up to at least a week before administering the induction chemotherapy treatment regimen to the subject, and then the induction chemotherapy regimen is administered to the subject concomitantly with the agent and/or gap junction blocker for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 2 weeks, at least 3 weeks, or at least a month, before ceasing administration of the induction chemotherapy regimen while continuing administration of the agent and/or gap junction blocker to the subject for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 2 weeks, at least 3 weeks, or at least a month. In some embodiments, the agent and/or gap junction blocker is administered to the subject for at least 2 days before administering an induction chemotherapy treatment regimen comprising 100 mg/kg cytarabine+3 mg/kg doxorubicin to the subject concomitantly with or without administering the agent and/or gap junction blocker for 3 days, followed by chemotherapy with 100 mg/kg cytarabine in the absence of doxorubicin concomitantly with or without the gap junction blocker for 2 days, followed by 2 weeks (14 days) of administration of the agent and/or gap junction blocker to the subject. In some embodiments, CBX is administered to the subject for at least 2 days before administering an induction chemotherapy treatment regimen comprising 100 mg/kg cytarabine+3 mg/kg doxorubicin to the subject concomitantly with or without administering CBX for 3 days, followed by chemotherapy with 100 mg/kg cytarabine in the absence of doxorubicin concomitantly with or without the CBX for 2 days, followed by 2 weeks (14 days) of administration of CBX to the subject. In some embodiments, administration of CBX or a gap junction blocker described herein comprises administering ascending and intermittent concentrations or doses of CBX or the gap junction blocker described herein over a period of time to the subject. For example, CBX or the gap-junction blocker can be administered at 10 mg/kg for at least 1 day, at least 2 days, at least 3 days, at least 4 days, at least 5 days, at least 6 days, or at least a week, followed by at least 1 day, at least 2 days, at least 3 days, at least 4 days, at least 5 days, or at least 1 week in the absence of administering CBX or the gap junction blocker, followed by administration of 20 mg/kg for at least 1 day, at least 2 days, at least 3 days, at least 4 days, at least 5 days, at least 6 days, or at least a week, followed by the absence of administration of CBX or the gap junction blocker for at least 1 day, at least 2 days, at least 3 days, at least 4 days, at least 5 days, at least 6 days, or at least a week, and then followed by administration of 30 mg/kg for at least 1 day, at least 2 days, at least 3 days, at least 4 days, at least 5 days, at least 6 days, or at least a week. It should be appreciated that the concentration or dosage of CBX or the gap junction blocker administered initially and at successive intervals after intermission of treatment can vary, as well as the escalation of the concentration or dose between treatment intervals. For example, the initial dose or concentration of CBX or the gap junction blocker can be 5 mg/kg, 10 mg/kg, 20 mg/kg, 25 mg/kg, 30 mg/kg, 35 mg/kg, 40 mg/kg, 45 mg/kg, or 50 mg/kg or more, and the escalation of the concentration or dose between intervals can be 5 mg/kg, 10 mg/kg, 15 mg/kg, 20 mg/kg, or 25 mg/kg. In addition, ascending and intermittent concentrations of doses of CBX or gap junction blocker can be administered over a variety of treatment intervals, e.g., 2, 3, 4, 5, 6, 7, 8, 9, 10, or as many as desired until the subject enters remission, to keep the subject in remission, or to further prolong survival of the patient, for example, by inducing the patient into remission or preventing the patient from relapsing from remission. In some embodiments, the treatment and intermission from treatment intervals can be more than a week, e.g., 2 weeks, 3 weeks, 4 weeks, 1 month, 2 months, 3 months, 6 months, or a year depending on the course of the disease in the subject. The aforementioned ascending and intermittent concentration or dosing schedules can be used when a subject is at a terminal state of the disease, for example, when leukemic cells are spread all over the subject's body, to prolong survival time of the subject.

As used herein, “treat,” “treatment,” “treating,” or “amelioration” when used in reference to a disease, disorder or medical condition, refers to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of, for example, acute myeloid leukemia, delay or slowing progression of acute myeloid leukemia, and an increased lifespan as compared to that expected in the absence of treatment.

In some embodiments, treating acute myeloid leukemia comprises inducing complete remission of acute myeloid leukemia in the subject. In some embodiments, the agent and/or gap junction blocker is administered to the patient for at least a day after complete remission is induced in the acute myeloid leukemia patient. In some embodiments, the agent and/or gap junction blocker is administered to the patient for at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 1 month, at least 2 months, at least 3 months, or more after complete remission is induced in the acute myeloid leukemia patient.

In some embodiments, treating acute myeloid leukemia comprises inducing complete remission of acute myeloid leukemia in the subject in the absence of a relapse risk due to residual leukemic cells in the subject's bone marrow or peripheral blood.

In some embodiments, the method further comprises evaluating the subject to determine if the subject has refractory or relapsed acute myeloid leukemia.

In some aspects, the disclosure provides a method of promoting survival of a subject suffering from acute myeloid leukemia, the method comprising administering to the subject an effective amount of an agent and/or gap junction blocker, thereby promoting survival of the subject. The method contemplates any gap junction blocker described herein. In some embodiments, the agent or gap junction blocker comprises an inhibitor of 11β-hydroxysteroid dehydrogenase (11β-HSD). In some embodiments, the agent or gap junction blocker comprises carbenoxolone or an analog thereof.

In some embodiments, the method further comprises administering an induction chemotherapy treatment regimen to the subject. In some embodiments, the induction chemotherapy comprises administering an antimetabolite agent and an anthracycline agent to the subject. In some embodiments, the antimetabolite agent comprises cytarabine. In some embodiments, the anthracycline agent comprises doxorubicin. In some embodiments, the induction chemotherapy comprises administering cytarabine and doxorubicin to the patient for a period of 5 days. In some embodiments, the induction chemotherapy comprises administering cytarabine and doxorubicin to the patient for a period of 3 days, followed by administering cytarabine alone to the patient for a period of 2 days. It should be appreciated that any of the administration or dosing schedules and/or treatment regiments described herein can be used with the method.

In some embodiments, the agent or gap junction blocker is administered to the subject for at least a day before administering the induction chemotherapy treatment regimen to the subject. In some embodiments, the agent or gap junction blocker is administered to the subject for at least a day before administering the induction chemotherapy treatment regimen to the subject concomitantly with the agent or gap junction blocker.

In some embodiments, the method further comprises selecting a subject suffering from or exhibiting a terminal state of acute myeloid leukemia. In some embodiments, the subject has advanced tumor metastasis. In some embodiments, the subject has a high tumor burden.

“Survival” refers to the subject remaining alive, and includes overall survival as well as progression free survival. “Overall survival” refers to the subject remaining alive for a defined period of time, such as 1 year, 2 years, 3 years, 4 years, 5 years, etc. from the time of diagnosis or treatment.

“Progression free survival” refers to the subject remaining alive, without the acute myeloid leukemia progressing or getting worse.

“Promoting survival” refers to enhancing one or more aspects of survival in a treated subject relative to an untreated subject (i.e., a subject not treated with a gap junction blocker, such as carbenoxolone), or relative to a subject treated with an approved chemotherapeutic agent alone in the absence of administration of a gap junction blocker (such as doxorubicin). In some embodiments, the gap junction blocker increases the subject's length of survival compared to the subject's length of survival in the absence of receiving the gap junction blocker. In some embodiments, the gap junction blocker increases the subject's likelihood of survival compared to the subject's likelihood of survival in the absence of receiving the gap junction blocker. In some embodiments, administration of the gap junction blocker (e.g., CBX) to the subject increases the subject's overall survival time by at least 1%, at least 2%, at least 3%, at least 4%, at least 5%, at least 10%, at least 15%, at least 20%, at least 25%, at least 30%, at least 40%, at least 50%, at least 60%, at least 70%, at least 80%, at least 90%, or more relative to subject's overall survival time in the absence of administration of the gap junction blocker and/or compared to chemotherapy treatment alone. In some embodiments, administration of the gap junction blocker (e.g., CBX) to the subject increases the subject's overall survival time by at least 1.1 fold, at least 1.2 fold, 1.3 fold, at least 1.4 fold, at least 1.5 fold, at least 1.6 fold, at least 1.7 fold, at least 1.8 fold, at least 1.9 fold, at least 2 fold, at least 3 fold, at least 4 fold, or, at least 5 fold or more relative to subject's overall survival time in the absence of administration of the agent or gap junction blocker and/or compared to chemotherapy treatment alone. In some embodiments, administration of the gap junction blocker (e.g., CBX) to the subject increases the subject's survival time by 1 day, 5 days, 10 days, 30 days, 2 months, 3 months, 4 months, 5 months, 6 months, 7 months, 8 months, 9 months, 10 months, 11 months, 1 year, 18 months, 2 years, 30 months, 3 years, 40 months, 4 years, 5 years, 6 years, 7 years, 8 years, 9 years, 10 years, 15 years, 20 years, 25 years, 30 years, 35 years, 40 years, 50 years, 55 years, 60 years, 65 years, 70 years, or 75 years or more relative to subject's overall survival time in the absence of administration of the agent or gap junction blocker and/or compared to chemotherapy treatment alone.

In one aspect, the disclosure provides a method of inducing complete remission in a subject having relapsed or refractory acute myeloid leukemia by selectively eradicating leukemic cells in the subject, the method comprising: (a) evaluating the subject to determine if the subject has relapsed or refractory acute myeloid leukemia; (b) administering to the subject an agent or gap junction blocker at least a day before administering an induction chemotherapy treatment regimen to the subject; and (c) administering to the subject an induction chemotherapy treatment regimen comprising an antimetabolite agent and an anthracycline agent for proscribed periods of time, thereby inducing complete remission in the subject by selectively eradicating leukemic cells in the subject.

Subjects

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein. In some embodiments, the subject suffers from acute myeloid leukemia.

In some embodiments, the subject is a patient presenting with acute myeloid leukemia. As used herein, “acute myeloid leukemia” encompasses all forms of acute myeloid leukemia and related neoplasms according to the World Health Organization (WHO) classification of myeloid neoplasms and acute leukemia, including all of the following subgroups in their relapsed or refractory state: Acute myeloid leukemia with recurrent genetic abnormalities, such as AML with t(8; 21)(q22; q22); RUNX1-RUNX1T1, AML with inv(16)(p13.1q22) or t(16; 16)(p13.1; q22); CBFB-MYH11, AML with t(9; 11)(p22; q23); MLLT3-MLL, AML with t(6; 9)(p23; q34); DEK-NUP214, AML with inv(3)(q21 q26.2) or t(3; 3)(q21; q26.2); RPN1-EVI1, AML (megakaryoblastic) with t(1; 22)(p13; q13); RBM15-MKL1, AML with mutated NPM1, AML with mutated CEBPA; AML with myelodysplasia-related changes; therapy-related myeloid neoplasms; AML, not otherwise specified, such as AML with minimal differentiation, AML without maturation, AML with maturation, acute myelomonocytic leukemia, acute monoblastic/monocytic leukemia, acute erythroid leukemia (e.g., pure erythroid leukemia, erythroleukemia, erythroid/myeloid), acute megakaryoblastic leukemia, acute basophilic leukemia, acute panmyelosis with myelofibrosis; myeloid sarcoma; myeloid proliferations related to Down syndrome, such as transient abnormal myelopoiesis or myeloid leukemia associated with Down syndrome; and blastic plasmacytoid dendritic cell neoplasm.

In some embodiments, the methods described herein further comprise selecting a subject diagnosed with acute myeloid leukemia, for example, based on the symptoms presented. Symptoms associated with acute myeloid leukemia are known to the skilled practitioner. For example, a patient can be diagnosed with acute myeloid leukemia if the subject presents with a myeloid neoplasm with 20% or more blasts in the peripheral blood or bone marrow.

In some embodiments, the methods described herein further comprise selecting a subject at risk of developing acute myeloid leukemia. For example, a subject can be selected as at risk of developing leukemia based on a family history of leukemias.

In some embodiments, a subject is selected as diagnosed with acute myeloid leukemia or at risk of developing acute myeloid leukemia based on a genetic mutation useful as a diagnostic or prognostic marker of myeloid neoplasms. Exemplary such markers include mutations of: JAK2, MPL, and KIT in MPN; NRAS, KRAS, NF1, and PTPN11 in MDS/MPN; NPM1, CEBPA, FLT3, RUNX1, KIT, WT1, and MLL in AML; and GATA1 in myeloid proliferations associated with Down syndrome (see Vardiman, et al., “The 2008 revision of the World Health Organization (WHO) classification of myeloid neoplasms and acute leukemia: rationale and important changes,” Blood 114(5), 937-951 (2009), incorporated herein by reference in its entirety).

In some embodiments, the methods described herein further comprise selecting a subject suspected of having acute myeloid leukemia. A subject suspected of having acute myeloid leukemia, for example, can be selected based on family history, diagnostic testing or based on the symptoms presented or a combination thereof.

In some embodiments, the methods described herein further comprise selecting a subject suffering from refractory or relapsed acute myeloid leukemia. As used herein, “relapsed acute myeloid leukemia” is defined as reappearance of leukemic blasts in the blood or greater than 5% blasts in the bone marrow after complete remission not attributable to any other cause. For subjects presenting with relapsed AML, more than 5% blasts on baseline bone marrow assessment is required. As used herein, “refractory acute myeloid leukemia” is defined as a failure to achieve a complete remission or complete remission with incomplete blood recovery after previous therapy. Any number of prior anti-leukemia schedules is allowed. As used herein, “complete remission” is defined as morphologically leukemia free state (i.e. bone marrow with less than 5% blasts by morphologic criteria and no Auer rods, no evidence of extramedullary leukemia) and absolute neutrophil count greater than or equal to 1,000/μL and platelets greater than 100,000/μL. As used herein, “complete remission with incomplete blood recovery” is defined as morphologically leukemia free state (i.e. bone marrow with less than 5% blasts by morphologic criteria and no Auer rods, no evidence of extramedullary leukemia) and neutrophil count less than 1,000/μL or platelets less than 100,000 μL in the blood.

In some embodiments, the methods described herein further comprise selecting a subject who relapses from complete remission of acute myeloid leukemia after receiving an induction chemotherapy treatment regimen.

Pharmaceutical Compositions

The disclosure contemplates compositions comprising the gap junction blockers and/or agents described herein (e.g., at least one chemotherapeutic agent subject to resistance by acute myeloid leukemia).

In some aspects, the disclosure provides a pharmaceutical composition comprising an effective amount of a gap junction blocker, and an effective amount of at least one chemotherapeutic agent subject to resistance by acute myeloid leukemia as described herein.

In some embodiments, a pharmaceutical composition comprises an effective amount of an agent or gap junction blocker, an effective amount of at least one chemotherapeutic agent subject to resistance by acute myeloid leukemia, and a pharmaceutically acceptable carrier, diluent, or excipient.

In some embodiments, the pharmaceutical composition includes an effective amount of a prophylactic treatment for hypertension, hypokalemia, and/or edemas.

The compositions comprising the agent or gap junction blocker and the at least one chemotherapeutic agent subject to resistance by acute myeloid leukemia can be used for treating acute myeloid leukemia as described herein. In some embodiments, the composition is useful for selectively eradicating leukemic cells in a subject without eradicating normal leukocytes in the subject. In some embodiments, the composition is useful for selectively eradicating leukemic cells in a subject with minimal eradication of normal leukocytes in the subject. In some embodiments, the composition is useful for selectively eradicating leukemic cells in a subject without eradicating normal cells in the subject. In some embodiments, the composition is useful for selectively eradicating leukemic cells in a subject with minimal eradication of normal cells in the subject. In some embodiments, the composition is useful for selectively eradicating leukemic cells in the subject while inducing proliferation of normal leukocytes in the subject. In some embodiments, the composition is useful for inducing complete remission of leukemia in the subject. In some embodiments, the composition is useful for inducing complete remission of acute myeloid leukemia in the subject. In some embodiments, the composition is useful for inducing complete remission of acute leukemia in the subject in the absence of a relapse risk due to residual leukemic cells in the subject's bone marrow or peripheral blood.

Formulation and Administration

The gap junction blockers and/or agents described herein can be administered alone or with suitable pharmaceutical carriers, and can be in solid or liquid form such as, tablets, capsules, powders, solutions, suspensions, or emulsions. As used herein, the term “administered” refers to the placement of an agent described herein, into a subject by a method or route which results in at least partial localization of the agent at a desired site. A gap junction blocker or agent described herein can be administered by any appropriate route which results in effective treatment in the subject, i.e. administration results in delivery to a desired location in the subject where at least a portion of the composition delivered. For a comprehensive review on drug delivery strategies see Ho et al., Curr. Opin. Mol. Ther. (1999), 1:336-3443; Groothuis et al., J. Neuro Virol. (1997), 3:387-400; and January, Drug Delivery Systems: Technologies and Commercial Opportunities, Decision Resources, 1998, content of all which is incorporate herein by reference. Exemplary routes of administration of the gap junction blockers and/or agents described herein (e.g., CBX) include, without limitation, intravenous administration, e.g., as a bolus or by continuous infusion over a period of time, intramuscular, intraperitoneal, intracerobrospinal, subcutaneous, intra-articular, intrasynovial, intrathecal, oral, topical, or inhalation routes. The gap junction blockers and/or agents can be formulated in pharmaceutically acceptable compositions which comprise a therapeutically-effective amount of the agent, formulated together with one or more pharmaceutically acceptable carriers (additives) and/or diluents, or excipients. The formulations can conveniently be presented in unit dosage form and may be prepared by any of the methods well known in the art of pharmacy. Techniques, excipients and formulations generally are found in, e.g., Remington's Pharmaceutical Sciences, Mack Publishing Co., Easton, Pa. 1985, 17th edition, Nema et al., PDA J. Pharm. Sci. Tech. 1997 51:166-171.

In some embodiments, the gap junction blockers and/or agents described herein can be administrated encapsulated within a nanoparticle (e.g., a lipid nanoparticle). In some embodiments, gap junction blockers and/or agents described herein can be administered encapsulated within liposomes. The manufacture of such liposomes and insertion of molecules into such liposomes being well known in the art, for example, as described in U.S. Pat. No. 4,522,811. Liposomal suspensions (including liposomes targeted to particular cells, e.g., endothelial cells) can also be used as pharmaceutically acceptable carriers.

The gap junction blockers and/or agents can be administrated to a subject in combination with other pharmaceutically active agents. Exemplary pharmaceutically active agents include, but are not limited to, those found in Harrison's Principles of Internal Medicine, 13^{th} Edition, Eds. T. R. Harrison et al. McGraw-Hill N.Y., NY; Physician's Desk Reference, 50^{th} Edition, 1997, Oradell N.J., Medical Economics Co.; Pharmacological Basis of Therapeutics, 8^{th} Edition, Goodman and Gilman, 1990; United States Pharmacopeia, The National Formulary, USP XII NF XVII, 1990, the complete contents of all of which are incorporated herein by reference. In some embodiments, the pharmaceutically active agent is a conventional treatment for acute myeloid leukemia. In some embodiments, the pharmaceutically active agent is a conventional treatment for an autoimmune or inflammatory condition. Chronic sub-cutaneous administration of a gap junction blocker described herein (e.g., CBX), in the absence of prophylactic treatment, could increase the frequency of deleterious side effects, for example, as a result of pseudo-hyperaldosteronism characterized by hypertension, hypokalemia and edemas (e.g., gastric edemas). Accordingly, in some embodiments, the pharmaceutically active agent comprises a prophylactic treatment, for example, to treat or prevent hypertension, hypokalemia, edemas, and other deleterious side effects caused by administration of the gap junction blocker (e.g., CBX). In some embodiments, such pharmaceutically active agent comprises an anti-mineralocorticoid or aldosterone inhibitor, such as an aldosterone receptor antagonist, e.g., eplerenone or spironolactone. In some embodiments, such pharmaceutically active agent comprises an angiotensin-converting-enzyme (ACE) inhibitor or other diuretic drug, e.g., thiazide diuretics, such as chlorothiazide, chlorthalidone, indapamide, hydrochlorothiazide, methylclothiazide, metolazone; loop diuretics, such as bumetamide, furosemide, ethacrynate, and torsemide; potassium sparing diuretics, such as amiloride hydrochloride, spironolactone, and triamterene; carbonic anhydrase inhibitors, such as acetazolamide, methazolamide, and osmotic diuretics, such as glycerin, isosorbide, mannitol IV, and urea. The skilled artisan will be able to select the appropriate conventional pharmaceutically active agent for treating any particular disease or disease subtype using the references mentioned above based on their expertise, knowledge and experience.

The agents and the other pharmaceutically active agent can be administrated to the subject in the same pharmaceutical composition or in different pharmaceutical compositions (at the same time or at different times). For example, a gap junction blocker and at least one chemotherapeutic agent subject to resistance by acute myeloid leukemia can be formulated in the same composition or in different compositions.

The pharmaceutical compositions can be included in a container, pack, or dispenser together with instructions for administration.

As used herein, “effective amount”, “effective amounts”, or “therapeutically effective amounts” means an amount of the agent (e.g., gap junction blocker) which is effective to selectively eradicate a majority or all of the leukemic cells (e.g., stem or progenitor cells) in a population of cells or a subject without eradicating, or minimally eradicating, normal cells (e.g., bone marrow or peripheral blood leukocytes) in the population or subject. Determination of a therapeutically effective amount is well within the capability of those skilled in the art. Generally, a therapeutically effective amount can vary with the subject's history, age, condition, sex, as well as the severity and type of the medical condition in the subject, and administration of other agents that inhibit pathological processes in the acute myeloid leukemia or autoimmune or inflammatory disorder.

Kits

The gap junction blockers and/or agents described herein can be provided in a kit. The kit includes (a) the agent, e.g., a composition that includes the agent, and (b) informational material. The informational material can be descriptive, instructional, marketing or other material that relates to the methods described herein and/or the use of the agent for the methods described herein. For example, the informational material describes methods for administering the gap junction blockers and/or agents to a subject for treating acute myeloid leukemia.

The informational material can include instructions to administer the gap junction blocker and/or agents described herein in a suitable manner, e.g., in a suitable dose, dosage form, or mode of administration. For example, due to its rapid action kinetics, gap junction blockers, such as CBX, induce selective apoptosis within hours of exposure of about 200 μM to leukemic cells. Similar plasma levels of CBX have been reported in ulcer patients taking 100 mg tablets, 3 times a day. Accordingly, in some embodiments the disclosure contemplates administering an effective amount of a gap junction blocker (e.g., CBX) to achieve plasma levels of about 200 μM. In some embodiments, the instructions recommend administering an effective amount of a gap junction blocker (e.g., CBX) to achieve plasma levels of about 200 μM. In some embodiments, the instructions recommend orally administering a gap junction blocker formulated as a tablet comprising 100 mg of the gap junction blocker (e.g., CBX) 3 times per day. The informational material can include instructions for selecting a suitable subject, e.g., a human, e.g., a human suffering from relapsed or refractory acute myeloid leukemia. The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is a link or contact information, e.g., a physical address, email address, hyperlink, website, or telephone number, where a user of the kit can obtain substantive information about the modulator and/or its use in the methods described herein. Of course, the informational material can also be provided in any combination of formats.

In addition to the agent or the composition, the kit can include other ingredients, such as a solvent or buffer, a stabilizer or a preservative, and/or a second agent for treating a condition or disorder described herein, e.g. acute myeloid leukemia. Alternatively, the other ingredients can be included in the kit, but in different compositions or containers than the agent. In such embodiments, the kit can include instructions for admixing the agent and the other ingredients, or for using the gap junction blocker together with the other ingredients.

The gap junction blocker and/or agents described herein can be provided in any form, e.g., liquid, dried or lyophilized form. It is preferred that the gap junction and/or agent be substantially pure and/or sterile. When the gap junction blocker and/or agent is provided in a liquid solution, the liquid solution preferably is an aqueous solution, with a sterile aqueous solution being preferred. When the gap junction blocker and/or agent is provided as a dried form, reconstitution generally is by the addition of a suitable solvent. The solvent, e.g., sterile water or buffer, can optionally be provided in the kit.

The kit can include one or more containers for the composition containing the agent. In some embodiments, the kit contains separate containers, dividers or compartments for the agent (e.g., in a composition) and informational material. For example, the agent (e.g., in a composition) can be contained in a bottle, vial, or syringe, and the informational material can be contained in a plastic sleeve or packet. In other embodiments, the separate elements of the kit are contained within a single, undivided container. For example, the agent (e.g., in a composition) is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agent (e.g., in a composition). For example, the kit includes a plurality of syringes, ampules, foil packets, or blister packs, each containing a single unit dose of the agent. The containers of the kits can be air tight and/or waterproof.

In some aspects, a kit comprises: a gap junction blocker, at least one chemotherapeutic agent subject to resistance by acute myeloid leukemia, and instructions for administering the agent or gap junction blocker and the at least one chemotherapeutic agent to a subject suffering from acute myeloid leukemia.

In some embodiments, the instructions further comprise directions for administering the at least one chemotherapeutic agent as part of an induction chemotherapy treatment regimen for the subject.

In some embodiments, the instructions further comprise directions for administering the agent or gap junction blocker, and the at least one therapeutic agent to induce complete remission of acute myeloid leukemia in the subject.

In some embodiments, the instructions further comprise directions for administering the agent or gap junction blocker, and the at least one therapeutic agent to induce complete remission of acute myeloid leukemia in the subject, without risk of relapse by completely eradicating leukemic cells in the subject.

In some embodiments, the instructions further comprise directions for administering the agent or gap junction blocker, and the at least one therapeutic agent to induce complete remission of acute myeloid leukemia in the subject by completely eradicating leukemic cells in the subject by inducing the leukemic cells to differentiate from proliferating, immortalized leukemic cells into short-lived, non-leukemic cells.

Agents

Without wishing to be bound by any theory, the agents (e.g., glycyrrhetinic acid derivatives) disclosed herein may eradicate leukemic cells by blocking or otherwise interfering with one or more of hemichannels and/or gap junctions, or blocking or interfering with one or more of connexins, pannexins and/or hydroxysteroid dehydrogenase, which are the building blocks of hemichannels and gap junctions. Accordingly, while certain aspects of the invention relate to the use of certain glycyrrhetinic acid derivatives (e.g., carbenoxolone and analogs thereof), it should be understood that the present inventions are not limited to such glycyrrhetinic acid derivatives, for example to eradicate leukemic cells. Rather, contemplated herein are any means of interfering with interactions with or between leukemic cells and thereby eradicating (e.g., selectively eradicating) leukemic cells or for blocking or interfering with hemichannels and/or gap junctions or connexins, pannexins and/or hydroxysteroid dehydrogenase.

For example, in certain aspects, the methods, kits and compositions disclosed herein may comprise any agents or compositions that are capable of or useful for blocking or otherwise interfering (e.g., selectively blocking or selectively interfering) with one or more of hemichannels, gap junctions, connexins, pannexins or hydroxysteroid dehydrogenase. Such agents or compositions may be selected from the group consisting of gap junction and hemichannels inhibitors such as glycyrrhizic acid, 18α-glycyrrhetinic acid, carbenoxolone, carbenoxolone derivatives, carbenoxolone analogs, fenamates, flufenemic acid, flufenemic acid derivatives, flufenemic acid analogs, heptanol, octanol, arachidonic acid, quinine, quinine derivatives (including mefloquine), connexin (Cx) fragments (including fragments from the extracellular domain of a connexin such as Connexin 43 or Connexin 30), connexin mimetic peptides including but not limited to Gap26 and Gap27, connexin inhibitors, connexin antibodies, connexin expression modulators such as clustered regularly-interspaced short palindromic repeats, CRISPR/Cas systems, siRNA, shRNA, miRNA and other oligonucleotides that regulate connexin expression (e.g., Nexagon®), Peptagon™, protein kinase C, Src, lysophosphatidic acid, inhibitors of arachidonic acid metabolism, niflumic acid, 5-nitro-2(3-phenylpropylamino)benzoic acid and a heavy metal such as lanthanum or gadolinium.

In some embodiments, the agents (e.g., carbenoxolone) disclosed herein preferentially bind to one or more of hemichannels and/or gap junctions of leukemic cells (e.g., leukemic stem cells), relative to normal cells. For example, in some embodiments, carbenoxolone preferentially binds to one or more of hemichannels and/or gap junctions of leukemic cells (e.g., leukemic stem cells) that is greater than 2 fold, 3 fold, 4 fold, 5 fold, six fold, 10 fold, 12 fold, 20 fold, 25 fold, 30 fold, 40 fold, 50 fold, 75 fold, 100 fold, 150 fold or greater than the binding to gap junctions or hemichannels of a normal cell. In some embodiments, carbenoxolone has a binding affinity for hemichannels and/or gap junctions of leukemic cells, that is greater than 2 fold, 3 fold, 4 fold, 5 fold, six fold, 10 fold, 12 fold, 20 fold, 25 fold, 30 fold, 40 fold, 50 fold, 75 fold, 100 fold, 150 fold or greater than the binding affinity with which it binds to the gap junctions or hemichannels of a normal cell.

In some embodiments, the agents (e.g., carbenoxolone) disclosed herein preferentially bind to one or more of connexins, pannexins and/or hydroxysteroid dehydrogenase of leukemic cells (e.g., leukemic stem cells), relative to normal cells. For example, in some embodiments, carbenoxolone preferentially binds to one or more of connexins, pannexins and/or hydroxysteroid dehydrogenase of leukemic cells that is greater than 2 fold, 3 fold, 4 fold, 5 fold, six fold, 10 fold, 12 fold, 20 fold, 25 fold, 30 fold, 40 fold, 50 fold, 75 fold, 100 fold, 150 fold or greater than the binding to connexins, pannexins and/or hydroxysteroid dehydrogenase of a normal cell. In some embodiments, carbenoxolone has a binding affinity for connexins, pannexins and/or hydroxysteroid dehydrogenase of leukemic cells (e.g., leukemic stem cells), that is greater than 2 fold, 3 fold, 4 fold, 5 fold, six fold, 10 fold, 12 fold, 20 fold, 25 fold, 30 fold, 40 fold, 50 fold, 75 fold, 100 fold, 150 fold or greater than the binding affinity with which it binds to the connexins, pannexins and/or hydroxysteroid dehydrogenase of a normal cell.

The disclosure contemplates the use of an agent or gap junction blocker alone, or in combination together with at least one additional chemotherapeutic agent, such as a chemotherapeutic agent subject to resistance by acute myeloid leukemia, in the methods, compositions, and kits described herein. The disclosure contemplates the use of any agents or gap junction blocker that is capable of selectively eradicating leukemic cells in a population of cells or subject, without eradicating, or minimally eradicating, normal cells (e.g., leukocytes) in the population or subject. Exemplary types of agents that can be used as a gap junction blocker include small organic or inorganic molecules; saccharines; oligosaccharides; polysaccharides; a biological macromolecule selected from the group consisting of peptides, proteins, peptide analogs and derivatives; peptidomimetics; nucleic acids selected from the group consisting of siRNAs, shRNAs, antisense RNAs, ribozymes, and aptamers; an extract made from biological materials selected from the group consisting of bacteria, plants, fungi, animal cells, and animal tissues; naturally occurring or synthetic compositions; and any combination thereof.

In some embodiments, the gap junction blocker comprises an inhibitor of 11β-hydroxysteroid dehydrogenase (11β-HSD). It should be appreciated that such inhibitor can be an inhibitor of 11β-HSD1, an inhibitor of 11β-HSD2, or an inhibitor both 11β-HSD1 and 11β-HSD2. In some embodiments, the gap junction blocker is selected from the group consisting of the following formulas I to III:

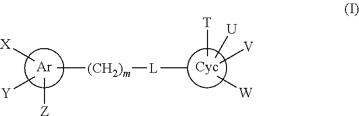

wherein

X₁ Y and Z each independently represent halogen, in particular, F, Cl, I or Br, C₁-C₆ alkyl, C₅-C₁₅ aryl or C₁-C₆ alkoxy,

n represents an integer from 1 to 10, in particular, from 1 to 4,

L represents an amide, amine, sulfonamide, ester, thioester or keto group,

T, U, V and W each independently represent an oxo, thio, ketone, thioketone, C₁-C₆ alkyl or C₁-C₆ alkanol group,

Ar represents an aromatic ring system, and

Cyc represents a cyclic ring system,

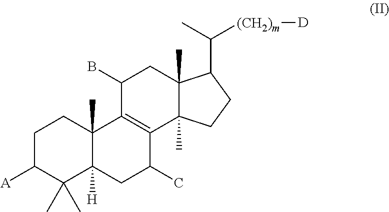

wherein

A represents a C₁-C₁₀ ester (C₁-C₁₀ alkyl-CO—O—), a C₁-C₁₀ amide (C₁-C₁₀ alkyl-CO—NH—), a C₁-C₁₀ ether or a C₁-C₁₀ ketone (C₁-C₁₀ alkyl-CO—) group,

B and C each independently represent an oxo group, a keto group, a C₁-C₆ alkanol group or a C₁-C₆ alkyl group,

m is an integer from 1 to 10, in particular, from 1 to 4, and

D is a group selected from COOR¹ or CONR²R³, wherein R¹, R² and R³ each independently represent H or a C₇-C₆ alkyl group,

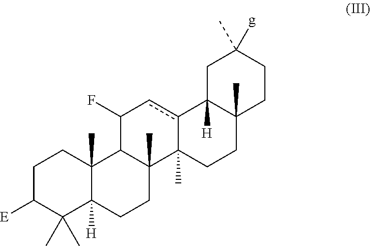

wherein

E represents an OH, a C₁-C₁₀ ester (C₁-C₁₀ alkyl-CO—O—), a C₁-C₁₀ amide (C₁-C₁₀ alkyl-CO—NH—), a C₁-C₁₀ ether (C₁-C₁₀—O—) or a C₁-C₁₀ ketone (C₁-C₁₀ alkyl-CO—) group,

F represents an oxo group, keto group, a C₁-C₆ alkanol group or a C₁— C₆ alkyl group, and

G is a group selected from COOR¹ or CONR²R³, wherein R¹, R² and R³ each independently represent H or a C₁-C₂₀ hydrocarbon group, in particular, a C₁-C₆ alkyl group.

In some embodiments, the gap junction blocker is 18-β-glycyrrhetinic acid or a derivative thereof. Exemplary derivatives of 18-β-glycyrrhetinic acid include, but are not limited to, glycyrrhizine, glycyrrhizinic acid, carbenoxolone, and 2-hydroxyethyl-18β-glycyrrhetinic acid amide.

In some embodiments, the gap junction blocker comprises carbenoxolone or an analog thereof. In some embodiments, the agent or gap junction blocker is not 18-β-glycyrrhetinic acid.

In some embodiments, the gap junction blocker is selected from the group consisting of heptanol, octanol, anadamide, fenamate, retinoic acid, oleamide, spermine, aminosulphates, halothane, enflurane, isoflurane, propofol, thiopental, glycyrrhetinic acid, quinine, 2-aminoethoxydiphenyl borate or pharmaceutically acceptable derivatives thereof, and any combination thereof. Exemplary pharmaceutically acceptable derivatives of heptanol include, but are not limited to, 1-heptanol, 2-heptanol, 3-heptanol, 4-heptanol, and combinations thereof. Exemplary pharmaceutically acceptable derivatives of fenamate include, but are not limited to, meclofenamic acid, niflumic acid, flufenamic acid, and combinations thereof. Exemplary pharmaceutically acceptable derivatives of glycyrrhetinic acid include, but are not limited to, hydrogen esters of glycyrrhetinic acid, salts of hydrogen esters of glycyrrhetinic acid, carbenoxolone, and combinations thereof. Methods of making hydrogen esters of glycyrrhetinic acid and salts of hydrogen esters of glycyrrhetinic acid are described in U.S. Pat. No. 3,070,623, incorporated by reference herein in its entirety. Exemplary pharmaceutically acceptable derivatives of quinine include, but are not limited to, quinidine, mefloquine, and combinations thereof.

In some embodiments, the gap junction blocker comprises carbenoxolone. In some embodiments, the agent or gap junction blocker is not 18-β-glycyrrhetinic acid.

In some embodiments, the gap junction blocker comprises an analog of carbenoxolone.

In some embodiments, the gap junction blocker comprises an inhibitor of a member of the connexin gap junction family. In some embodiments, the gap junction blocker comprises an inhibitor of connexin Cx40.1. In some embodiments, the gap junction blocker comprises an inhibitor of connexin Cx30.2. In some embodiments, the gap junction blocker comprises an inhibitor of connexin Cx31.1. In some embodiments, the gap junction blocker comprises an inhibitor of connexin Cx36. In some embodiments, the gap junction blocker comprises an inhibitor of connexin Cx45. In some embodiments, the gap junction blocker comprises an inhibitor of connexin Cx47. In some embodiments, the gap junction blocker comprises an inhibitor of connexin Cx32. In some embodiments, the gap junction blocker comprises an inhibitor of connexin Cx50. In some embodiments, the gap junction blocker comprises an inhibitor of connexin Cx30.3. In some embodiments, the gap junction blocker comprises an inhibitor of connexin Cx31. In some embodiments, the gap junction blocker comprises an inhibitor of connexin Cx26. In some embodiments, the gap junction blocker comprises an inhibitor of connexin Cx40. In some embodiments, the gap junction blocker comprises an inhibitor of connexin Cx37. In some embodiments, the gap junction blocker comprises an inhibitor of connexin Cx46. In some embodiments, the gap junction blocker comprises an inhibitor of connexin Cx43. In some embodiments, the gap junction blocker comprises an inhibitor of connexin Cx30. Exemplary inhibitors of the connexin gap junction family members listed above include, but are not limited to, extracellular Ca2+, carbenoxolone, flufenamic acid, and octanol. Other suitable inhibitors of the connexin gap junction family members listed above would be apparent to the skilled artisan.

The disclosure contemplates the use of any chemotherapeutic agent that is useful for treating cancer (e.g., leukemia). Exemplary chemotherapeutic agents that can be administered in combination with the gap junction blockers of the present invention (e.g., agents that disrupt intercellular communications) include alkylating agents (e.g. cisplatin, carboplatin, oxaloplatin, mechlorethamine, cyclophosphamide, chorambucil, nitrosureas); anti-metabolites (e.g. methotrexate, pemetrexed, 6-mercaptopurine, dacarbazine, fludarabine, 5-fluorouracil, arabinosycytosine, capecitabine, gemcitabine, decitabine); plant alkaloids and terpenoids including vinca alkaloids (e.g. vincristine, vinblastine, vinorelbine), podophyllotoxin (e.g. etoposide, teniposide), taxanes (e.g. paclitaxel, docetaxel); topoisomerase inhibitors (e.g. notecan, topotecan, amasacrine, etoposide phosphate); antitumor antibiotics (dactinomycin, doxorubicin, epirubicin, and bleomycin); ribonucleotides reductase inhibitors; antimicrotubules agents; and retinoids. (See, e.g., Cancer: Principles and Practice of Oncology (V. T. DeVita, et al., eds., J. B. Lippincott Company, 9^{th} ed., 2011; Brunton, L., et al. (eds.) Goodman and Gilman's The Pharmacological Basis of Therapeutics, 12^{th} Ed., McGraw Hill, 2010).

The compositions, methods, and kits described herein contemplate the use of at least one chemotherapeutic agent subject to resistance by acute myeloid leukemia. The at least one chemotherapeutic agent may be subject to drug resistance by acute myeloid leukemia due to any drug resistance mechanism. In some embodiments, the at least one chemotherapeutic agent is subject to stroma-mediated drug resistance. As used herein, stroma-mediated drug resistance refers to chemoresistance exhibited by acute myeloid leukemia due to heterotypic interactions between the leukemic cells and stromal cells. In some embodiments, the at least one chemotherapeutic agent subject to resistance by acute myeloid leukemia comprises an antimetabolite agent. In some embodiments, the at least one chemotherapeutic agent subject to resistance by acute myeloid leukemia comprises cytarabine. In some embodiments, the at least one chemotherapeutic agent subject to resistance by acute myeloid leukemia comprises an anthracycline agent. In some embodiments, the at least one chemotherapeutic agent subject to resistance by acute myeloid leukemia comprises doxorubicin. In some embodiments, the at least one chemotherapeutic agent subject to resistance by acute myeloid leukemia comprises an antimetabolite agent and an anthracycline agent. In some embodiments, the at least one chemotherapeutic agent subject to resistance by acute myeloid leukemia comprises cytarabine and the anthracycline agent comprises doxorubicin. It should be appreciated that administration of a gap junction blocker described herein (e.g., CBX) selectively eradicates leukemic cells by, in part, overcoming chemoresistance exhibited by leukemic cells, such as stroma-mediated chemoresistance.

### SOME DEFINITIONS

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As used herein the term “comprising” or “comprises” is used in reference to compositions, methods, kits and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, kits and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages may mean±1%.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

All patents and other publications identified are expressly incorporated herein by reference for the purpose of describing and disclosing, for example, the methodologies described in such publications that might be used in connection with the disclosure. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

To the extent not already indicated, it will be understood by those of ordinary skill in the art that any one of the various embodiments herein described and illustrated may be further modified to incorporate features shown in any of the other embodiments disclosed herein.

The following example illustrates some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### EXAMPLES

### Example 1

### Gap Junction Intercellular Communication Regulate Leukemic Cell Survival and Drug Resistance

Some fusion proteins encoded by genetic translocations in human leukemias, including those involving the mixed lineage leukemia (MLL) gene, have been reported to impart leukemia stem cell properties on committed hematopoietic progenitors (Huntly, et al., Cancer Cell 6, 587-96 (2004); Cozzio, et al., Genes & Development 17, 3029-35 (2003); and (So, et al., Cancer Cell 3, 161-71 (2003)). Furthermore, introduction of these altered alleles into normal bone marrow cells induces AML in mouse models of the disease. Such AML models recapitulate the human disease phenotype and display stem cell-like properties that demonstrate the ability to serially colonize in-vitro, and the ability to confer an AML phenotype that can be serially transplanted in vivo (Huntly, et al. (2004) and Krivtsov, et al., Nature 442, 818-22 (2006)).

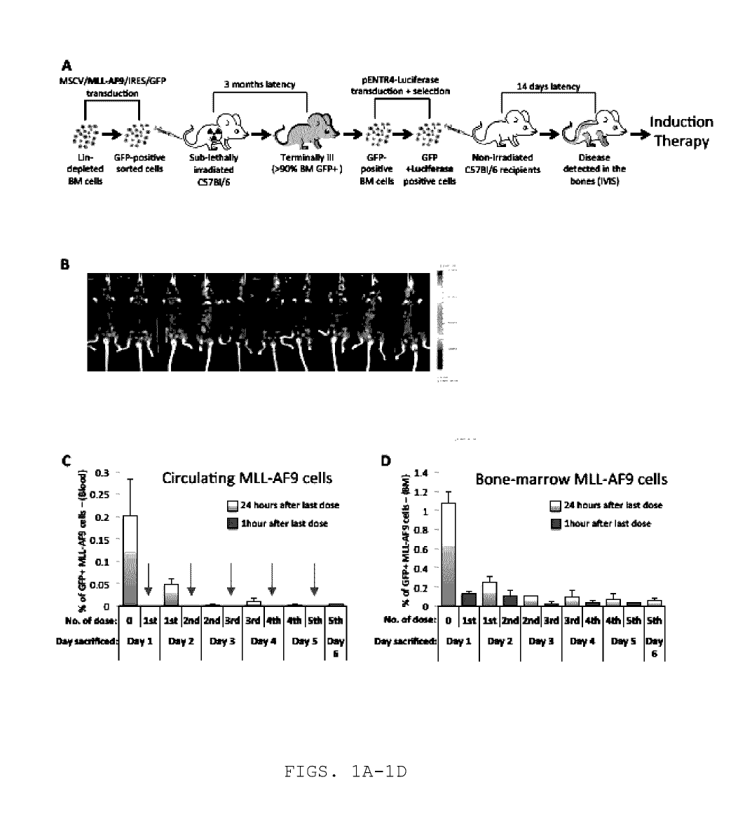

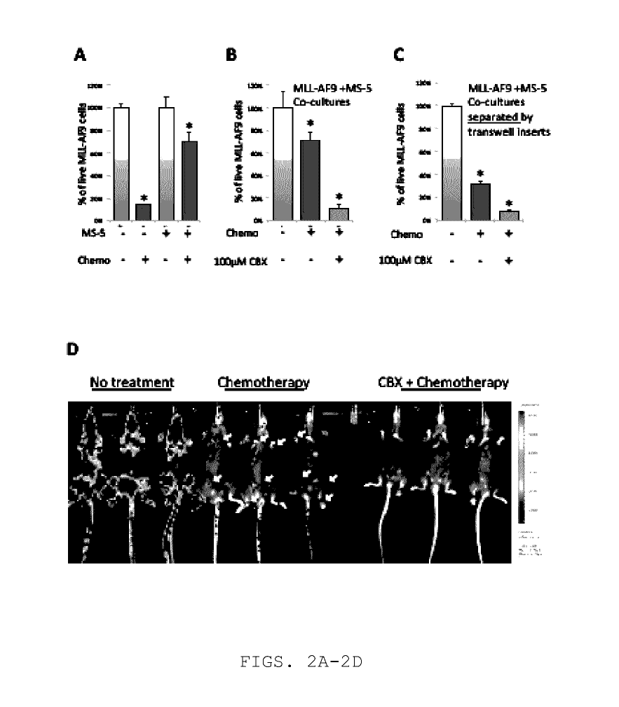

We first studied the immediate response of AML cells to induction chemotherapy in a mouse model of human MLL-AF9 leukemia. MLL-AF9 is a fusion protein, encoded by the t(9; 11)(p22; q23) translocation (Krivtsov, et al. 2006 and Sykes, et al., Cell 146, 697-708 (2011)), present in leukemic blasts derived from patients with AML and is associated with poor prognosis (Schoch, et al., Blood 102, 2395-402 (2003)). In order to generate primary MLL-AF9 leukemic cells for our experiments, we introduced the MLL-AF9 oncogene via retroviral transduction into lineage depleted bone marrow cells from B6.SJL mice, as depicted in FIG. 1A. Sorted GFP positive cells were then intravenously injected into sub-lethally irradiated C57B16 recipients and MLL-AF9 GFP-positive leukemic cells were harvested 3 months later, at a terminal stage of disease. In order to synchronize the timing of the treatment and to visualize disease progression, we transduced primary MLL-AF9 GFP-positive cells with Luciferase lentivirus. Then, we selected Luciferase expressing cells with antibiotics and transplanted 1 million double positive leukemic cells into non-irradiated recipients and monitored disease progression by whole body bioluminescence imaging (FIGS. 1A and 1B). In the clinic, newly diagnosed AML patients are being treated with induction chemotherapy (Cytarabine combined with anthracycline) to enter complete remission (Pui et al. 2011 and Burnett et al. 2011) and a similar outcome has been described in mouse model of human AML by a 5-days treatment regimen of combined Cytarabine and Doxorubicin for 3 days, proceeded with Cytarabine alone for additional 2 days (Zuber, et al., Genes & Development 23, 877-89 (2009)). 14 days after transplantation into non-irradiated recipients, MLL-AF9 cells were detected in the bones of recipients (FIG. 1B) and mice were further stratified by % of circulating GFP-positive prior to treatment. Our kinetics experiments revealed a very fast response to induction chemotherapy and GFP-positive MLL-AF9 cell were not detected in the circulation 1-hour after the 1st dose, by flow cytometry (FIG. 1C). 24-hours after the 1st dose we could detect GFP-positive circulating MLL-AF9 cells, but they disappeared again 1-hour after the 2nd dose. Similar pattern was recorded with the 3rd, 4th and 5th doses (FIG. 1C). Surprisingly, the levels of bone marrow MLL-AF9 cells were dramatically reduced 1 hour after every dose in the course of the 5-day regimen (FIG. 1D). We hypothesized that this may be reflective of functional changes beyond that of cell death. Indeed we found that treating primary AML cells in vitro with effective doses of combined chemotherapy did not induce cell death.

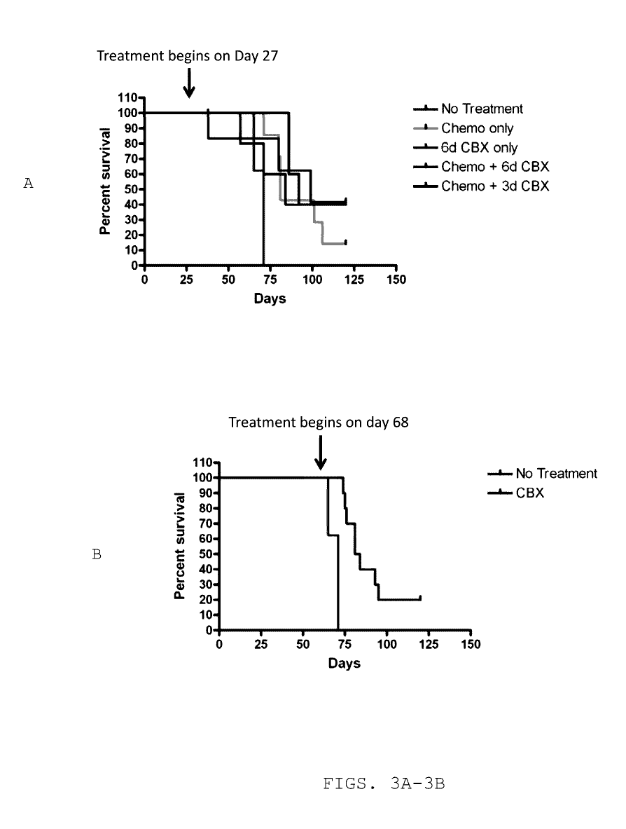

We hypothesize that perhaps chemotherapy treatment led to a different in vivo localization of cells. If true, cell-cell connections could be occurring and might contribute to AML cell survival. To test this hypothesis, we evaluated the response of MLL-AF9 leukemic cells to chemotherapy in vitro, with and without supporting bone marrow stromal cells using time-lapse video microscopy combined with flow cytometry. Only high dose of 50 nM Cytarabine together with 20 nM Doxorubicin (Pardee, et al., Experimental Hematology 39, 473-485 (2011)), resulted in low resistance and ˜85% cell death, 16 hours post induction (FIG. 2A). MS-5 are murine bone-marrow stromal cells that were previously shown to support hematopoietic stem and progenitor cells (Itoh, et al., Experimental Hematology 21, 145-153 (1989) and Schajnovitz, et al., Nature Immunology 12, 391-8 (2011)). As expected, co-culture of MLL-AF9 cells with BM supporting MS-5 stromal cells, resulted in increased resistance (˜70%) to combined therapy with only ˜30% cell death (FIG. 2A). This suggests that direct interactions between the leukemic cells and the stromal cells facilitate drug resistance. In order to distinguish between Stromal-leukemic (heterotypic) interactions and leukemic-leukemic (homotypic) interactions, we separated the MLL-AF9 from the stroma by transwell inserts, to restrict heterotypic interactions while allowing homotypic interactions. We found that heterotypic interactions are the major cause of resistance, but not entirely, as ˜30% (compare to ˜15% resistance without stroma) of the MLL-AF9 cells were still resistant, although physically separated from the stroma (FIG. 2C).

Since adhesion interactions of hematopoietic cells are commonly accompanied with intercellular communication, we then tested the potential role of gap junctions activity in drug resistance. Gap junction intercellular channels, which are homo- and hetero-hexamers of connexin proteins, facilitate intercellular communication between contacting cells via the passage of secondary messengers, such as calcium and cAMP¹⁷. Carbenoxolone (CBX) is a potent broad range gap junctions inhibitor which efficiently blocks cell-cell communication at 100 μM without affecting cell viability (Schajnovitz et al. 2011). Blocking gap junctions activity in the coculture system by 100 μM CBX, 20 minutes before induction chemotherapy, reversed the resistance and almost 90% of the ML-AF9 cells were eradicated (FIG. 2B). Gap junction blockade in co-cultures separated with transwell inserts, resulted in more than 90% eradication (FIG. 2C), suggesting that homotypic intercellular communication also contributes to acquired drug resistance.

Following these encouraging results, we tested the effect of systemic gap junction inhibition combined with chemotherapy in mice. 25 mg/kg CBX was administrated to sick mice, 24 hours before induction chemotherapy and throughout the 5-days treatment. 1 week after treatment, mice were imaged and then sacrificed to evaluate treatment outcome. As depicted in FIG. 2D, mice treated with chemotherapy alone entered complete remission but minimal residual cells could be detected in the bones (white arrows). Strikingly, mice that were treated with chemotherapy and a gap junction blocker were entirely leukemia free, with no detectable leukemia cells by luciferase imaging or by flow cytometry.

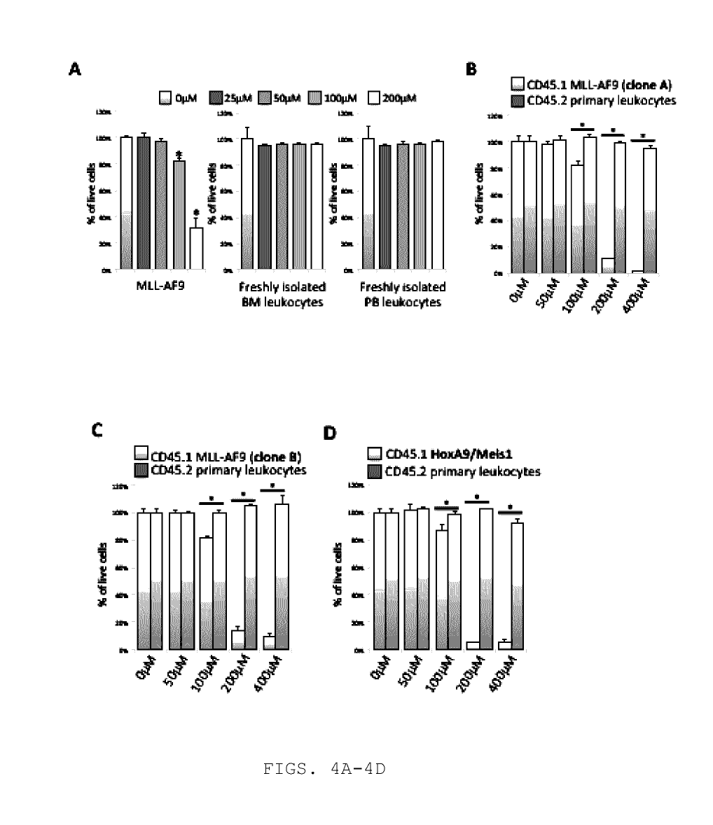

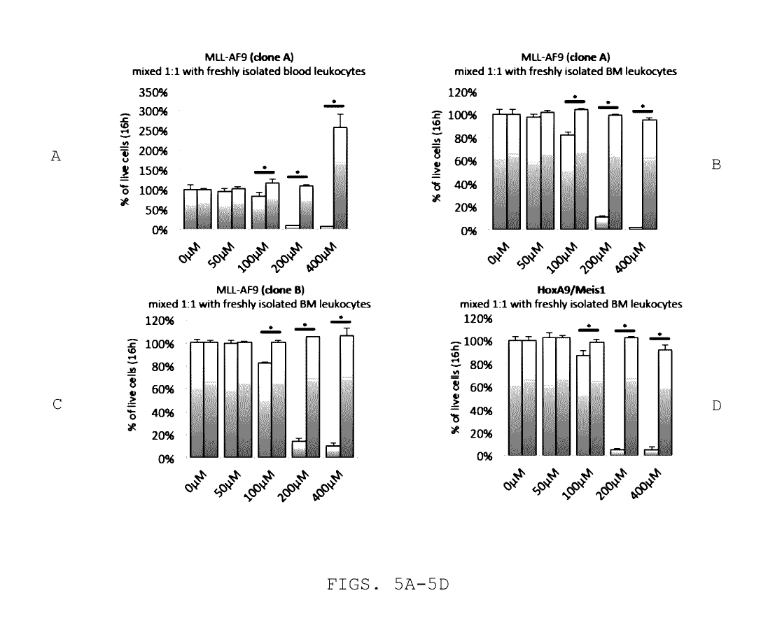

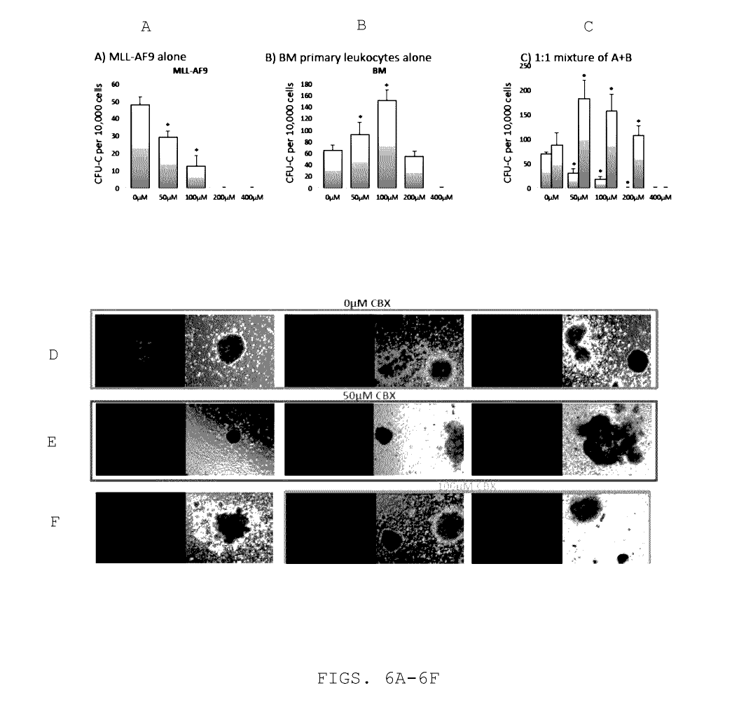

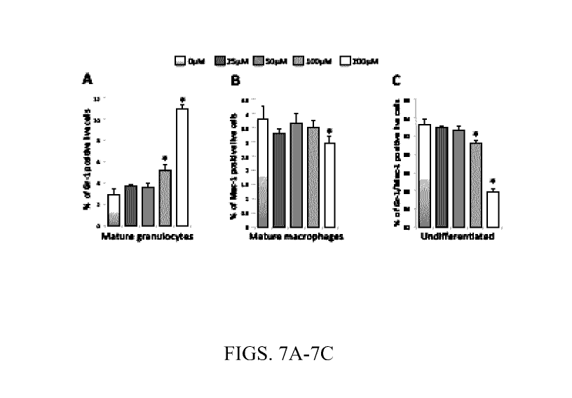

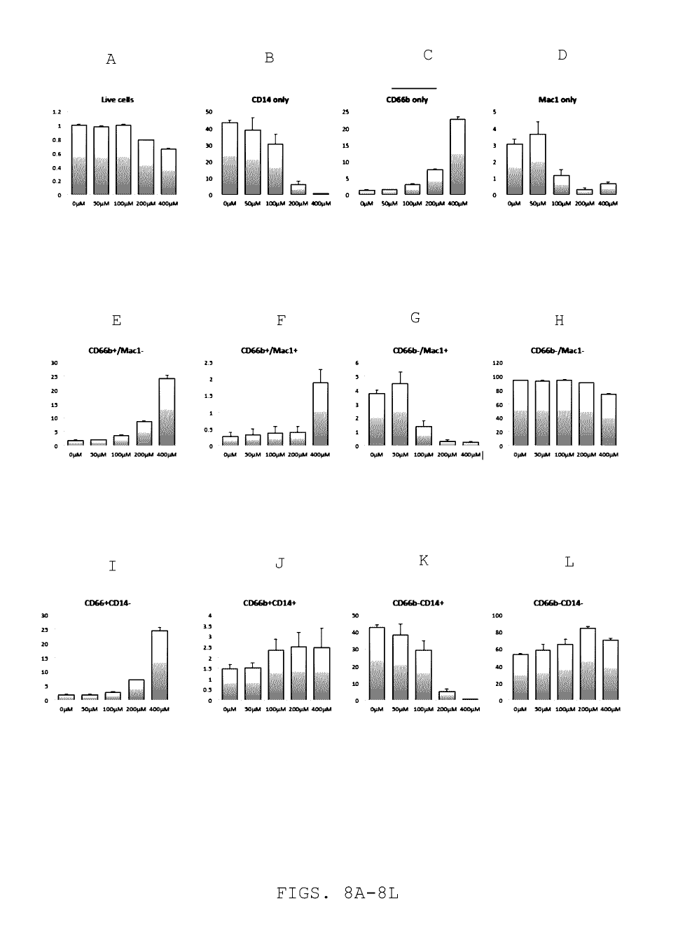

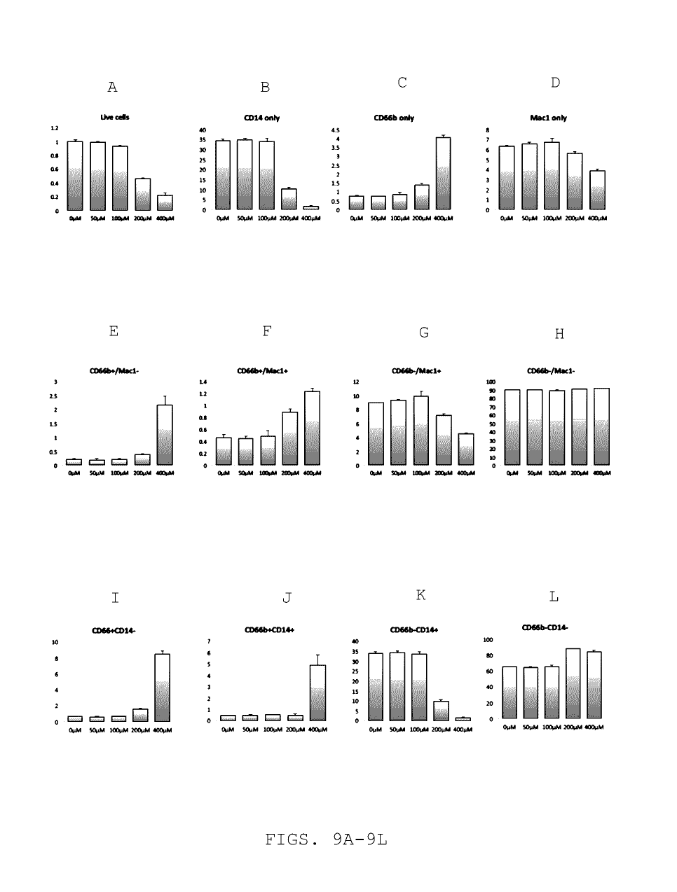

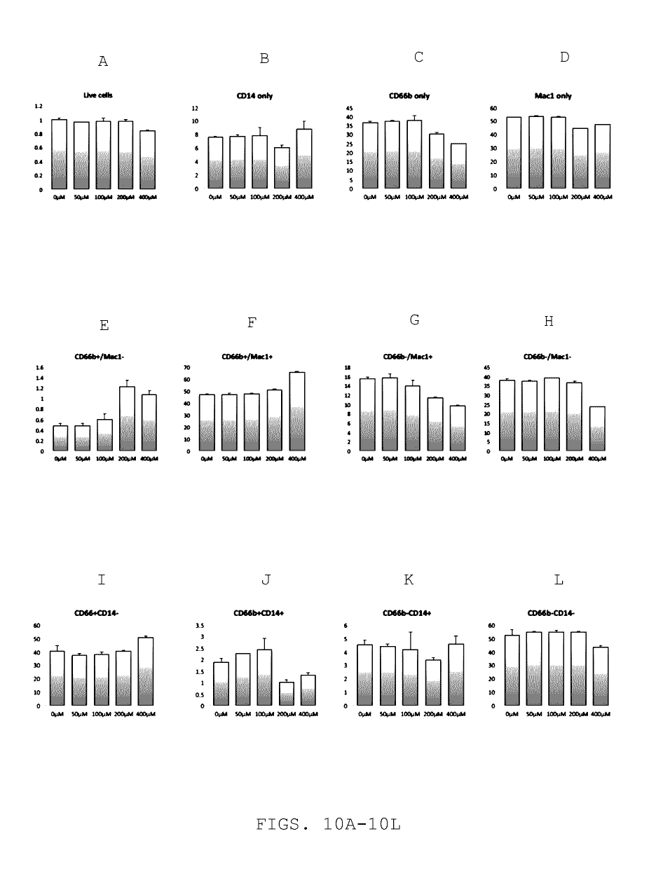

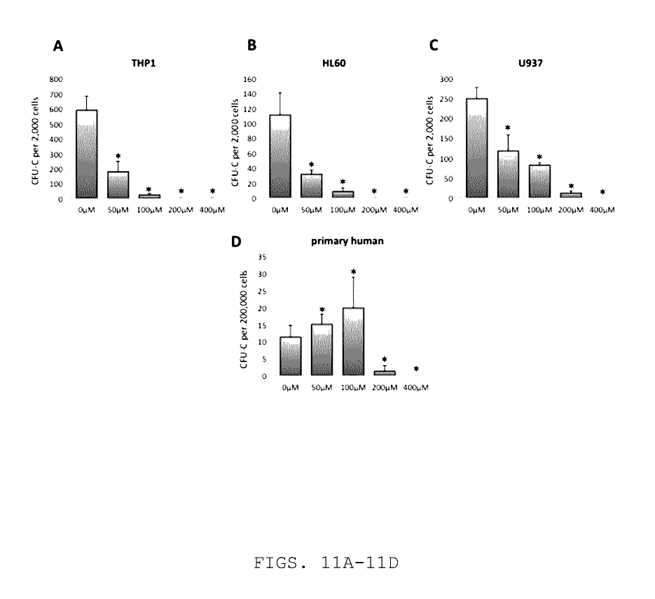

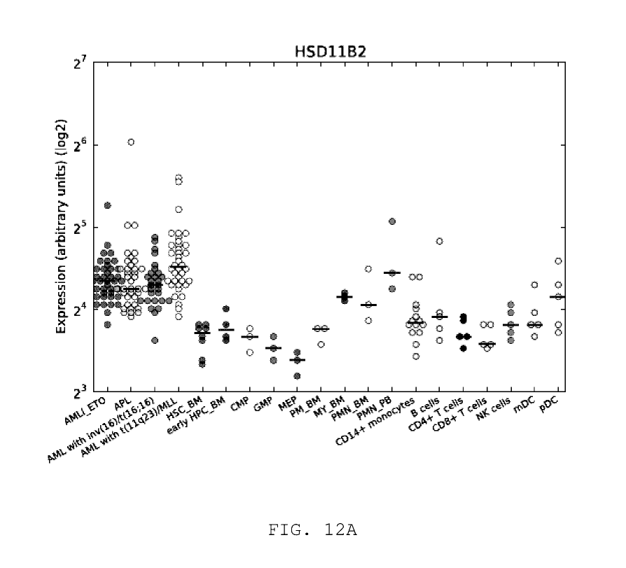

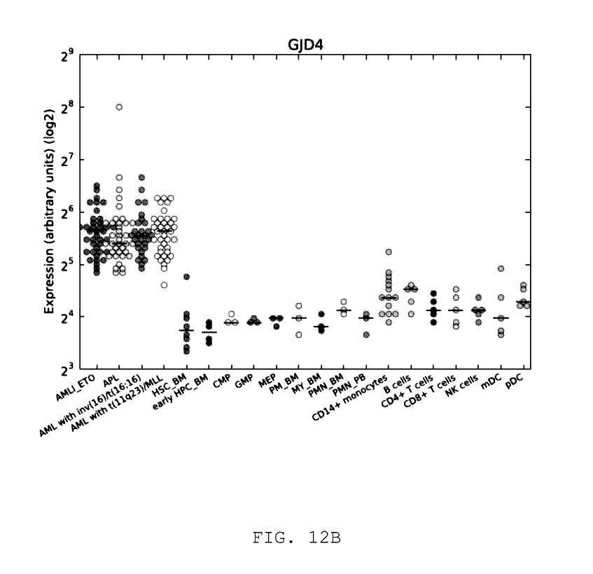

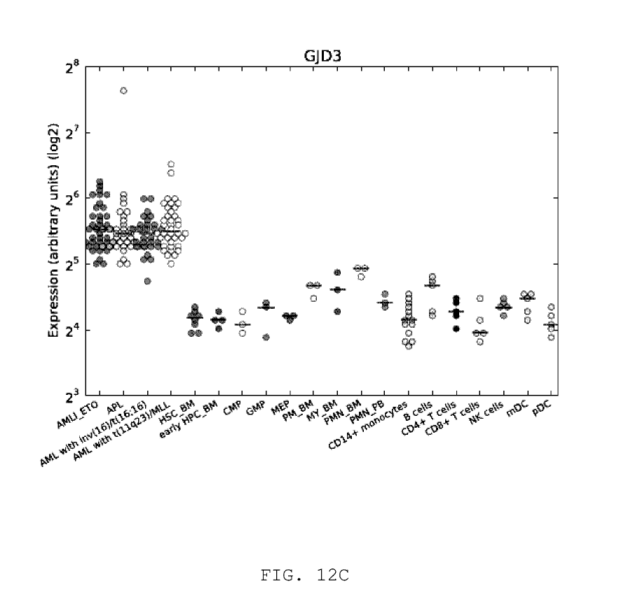

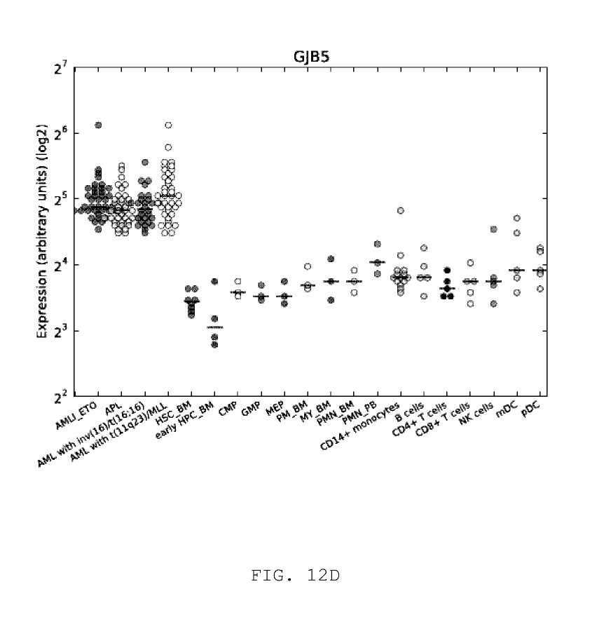

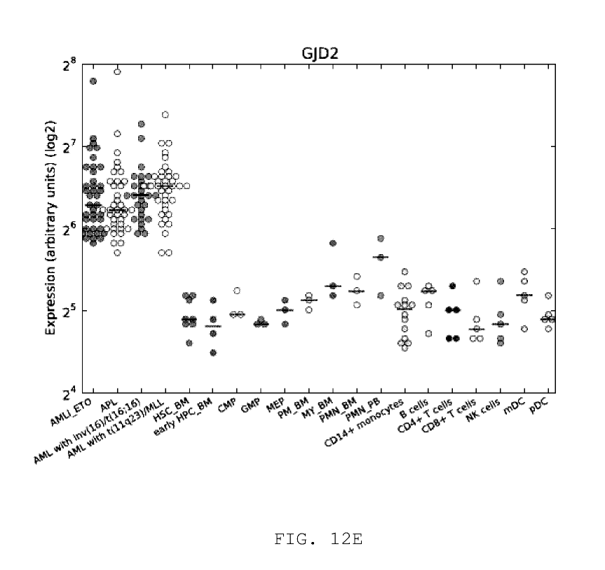

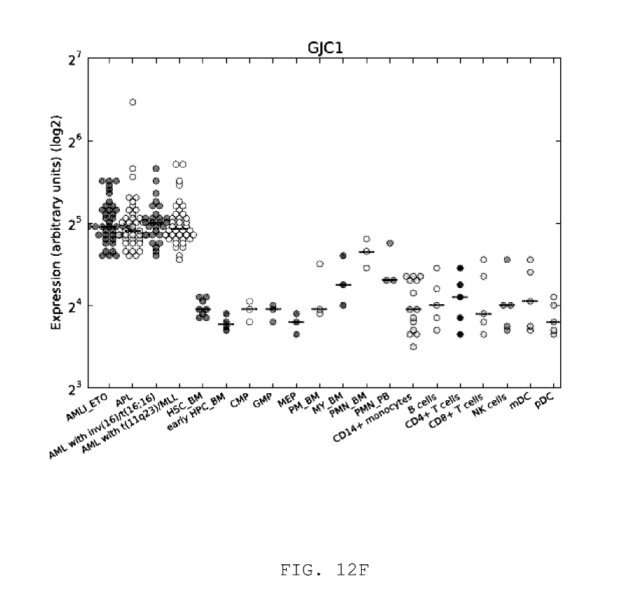

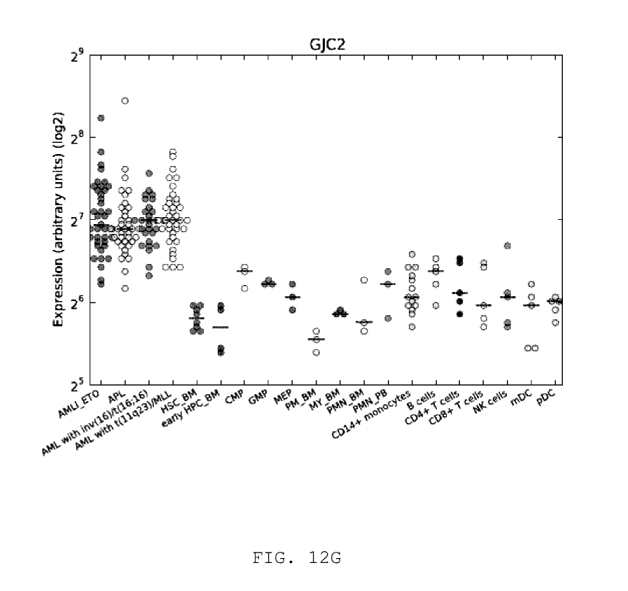

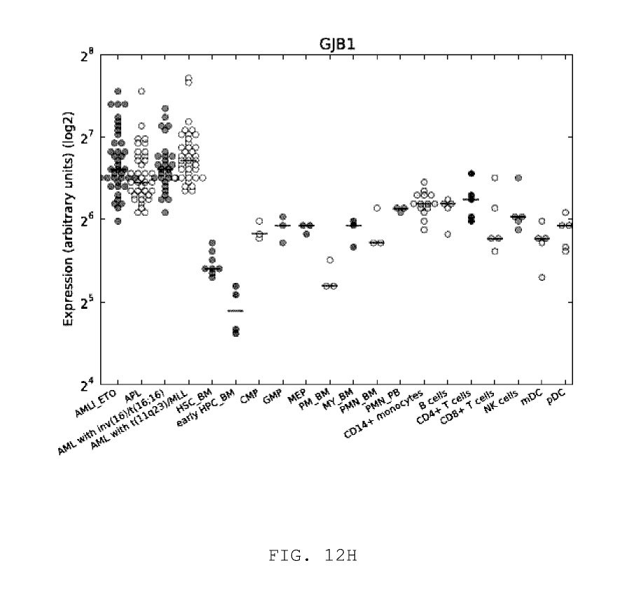

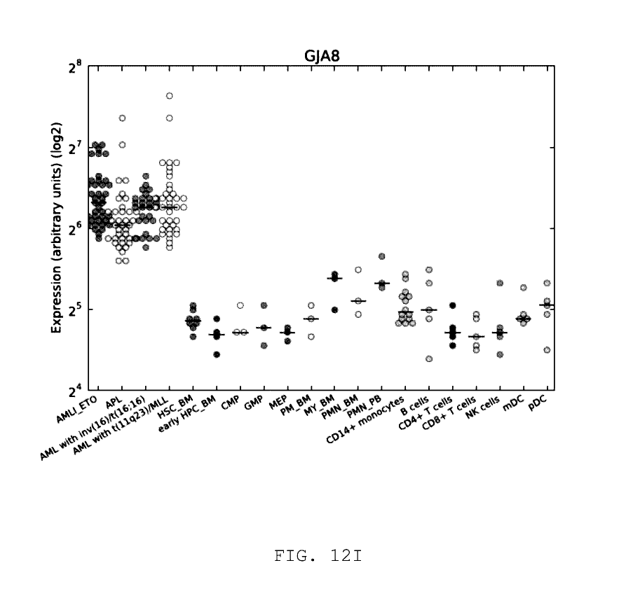

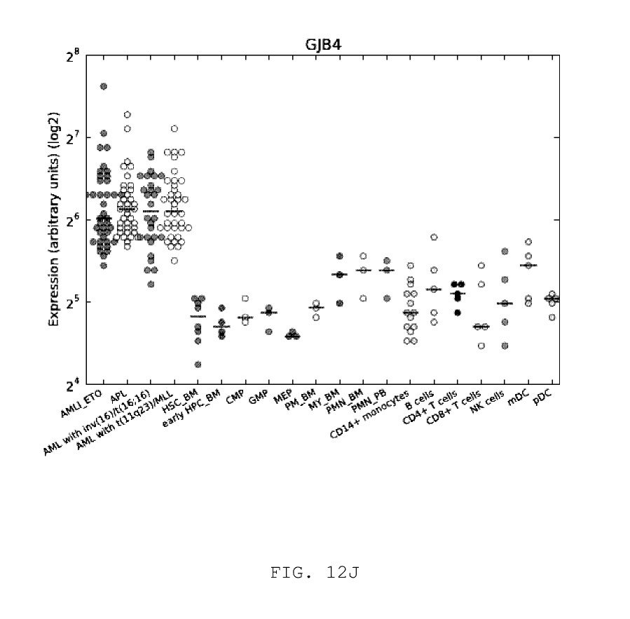

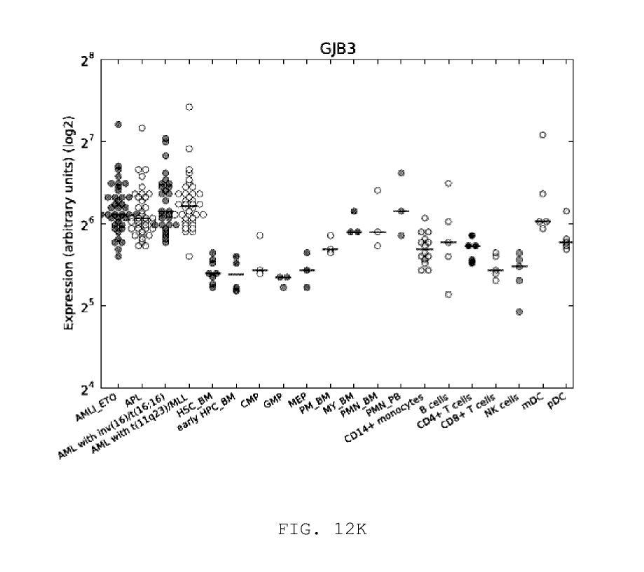

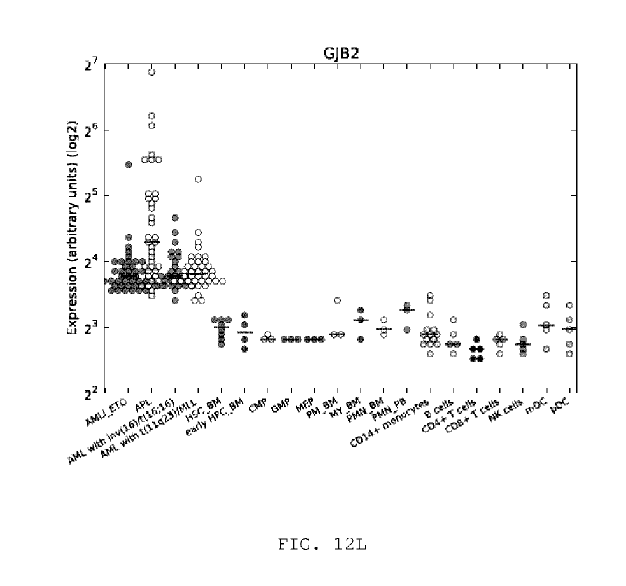

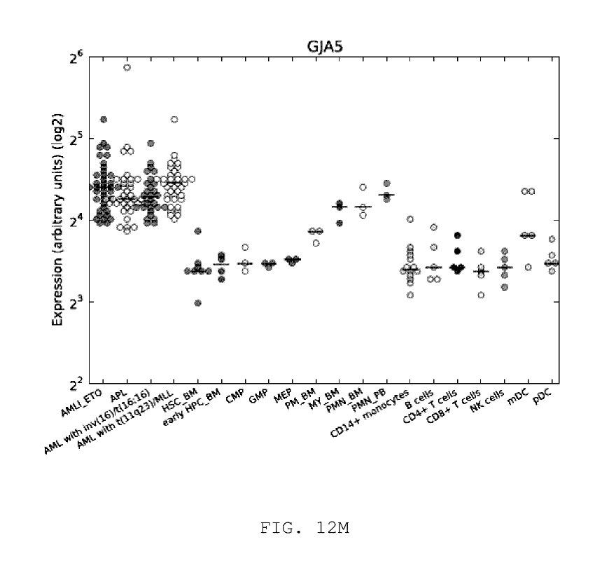

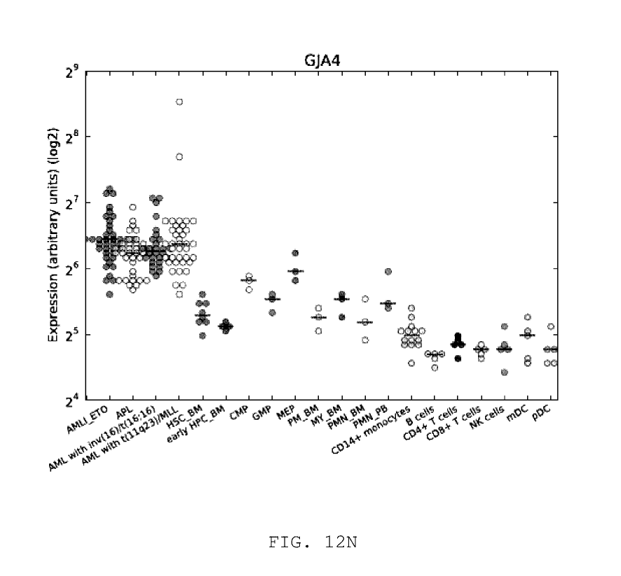

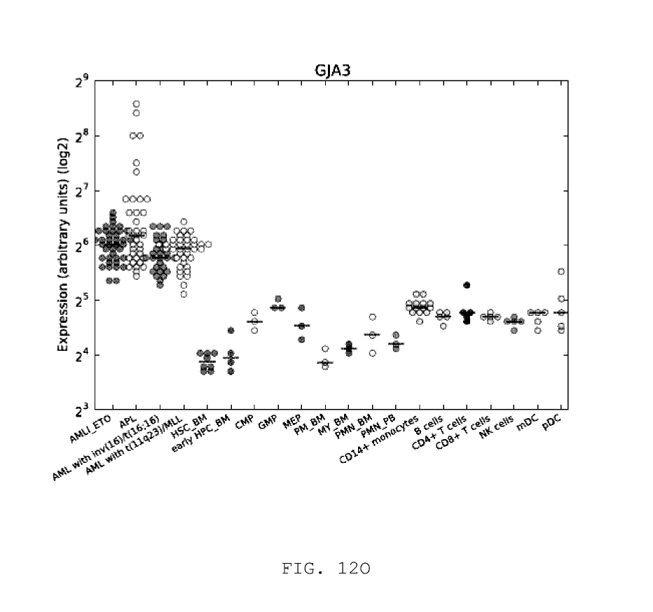

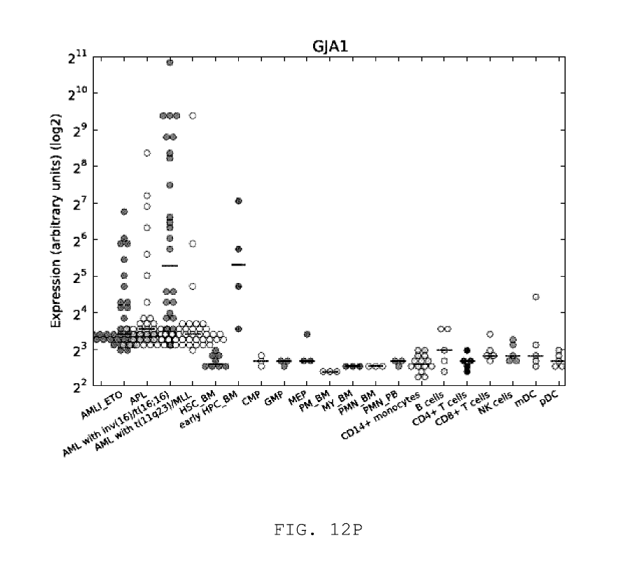

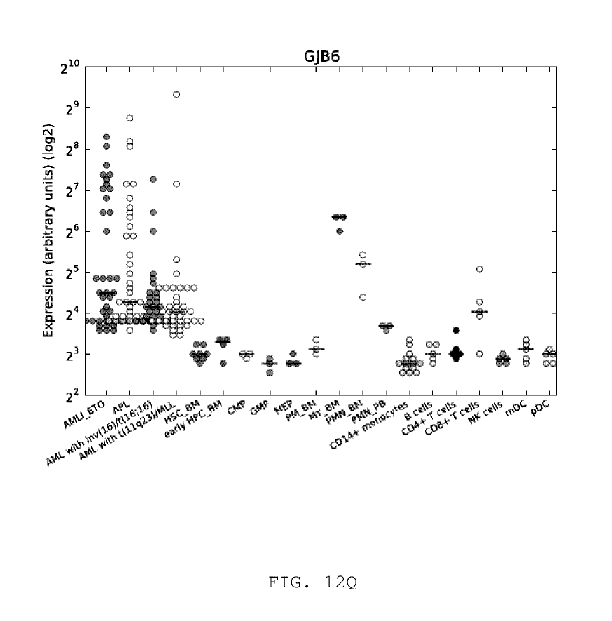

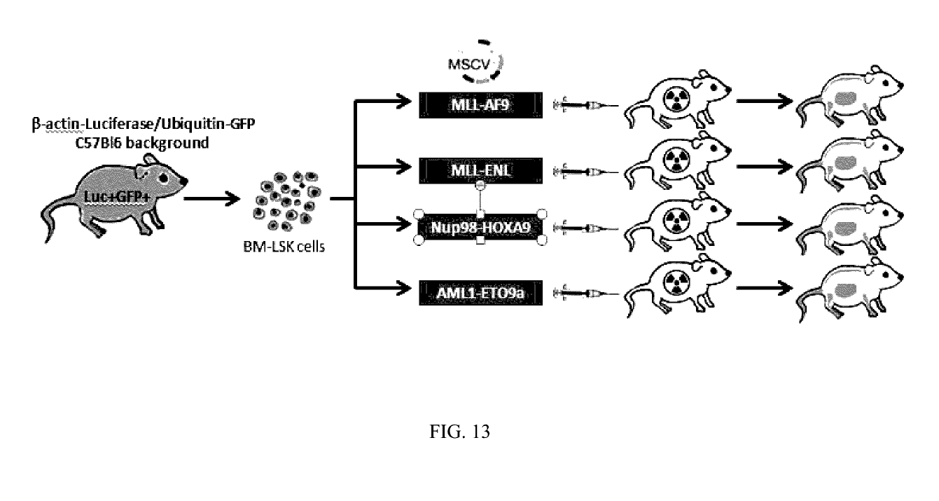

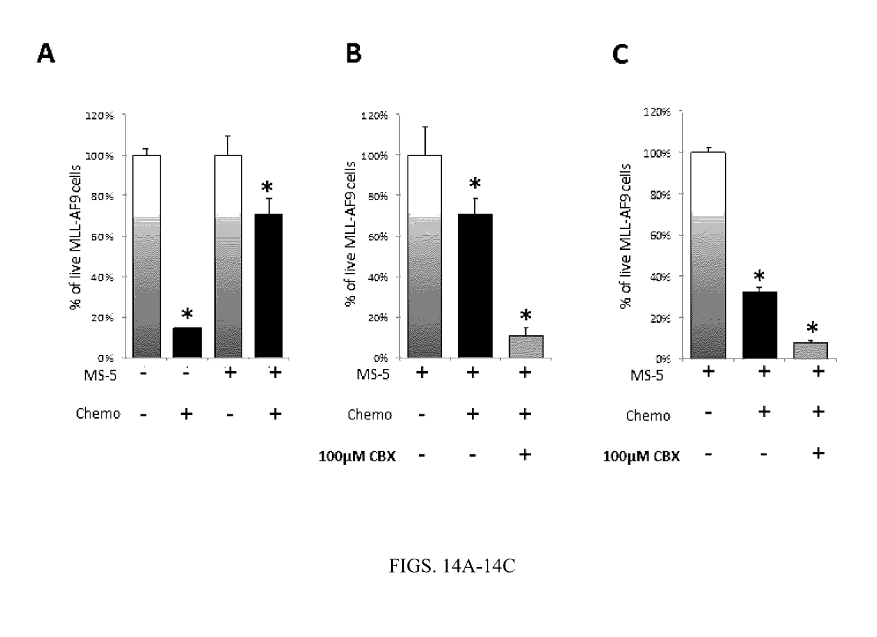

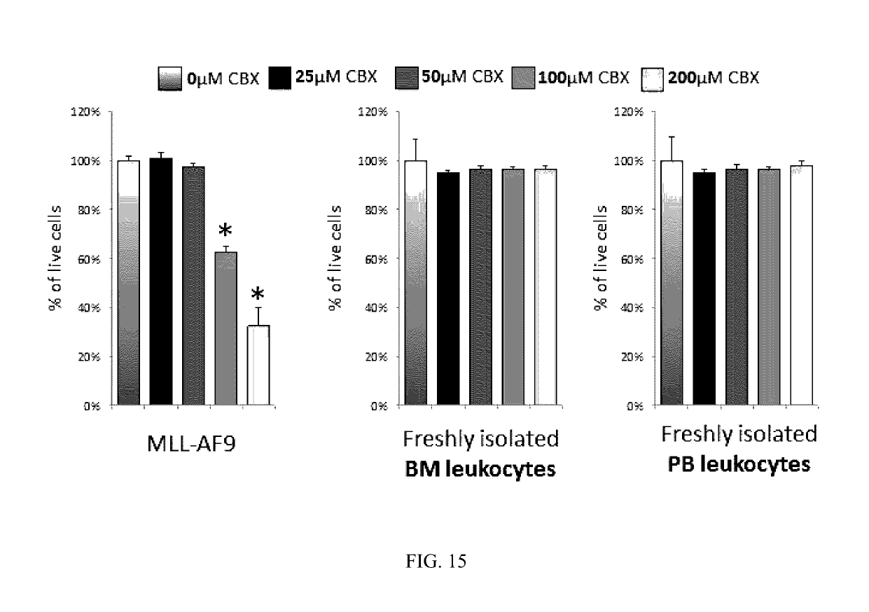

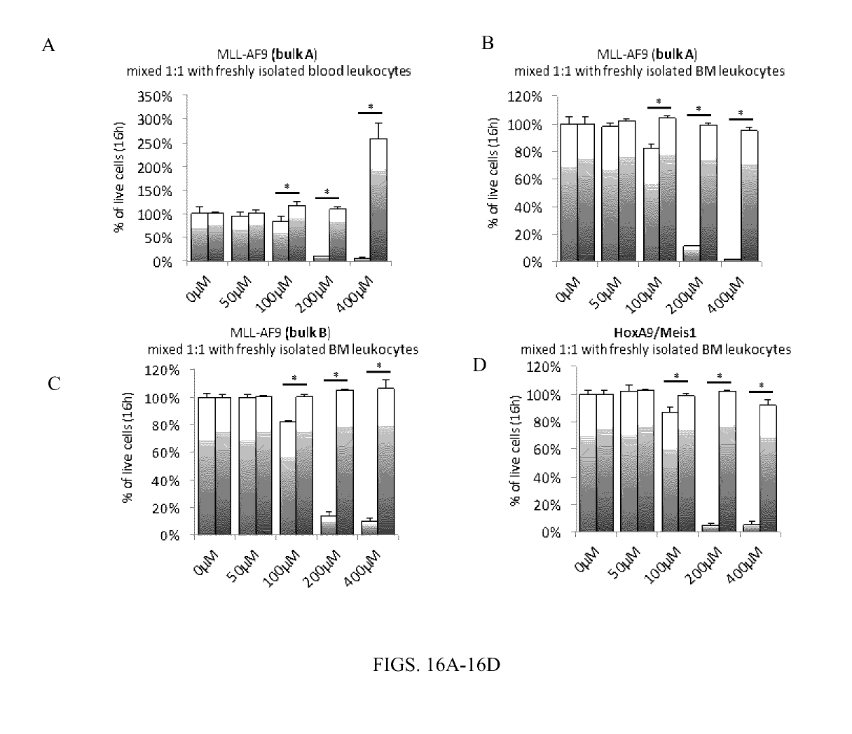

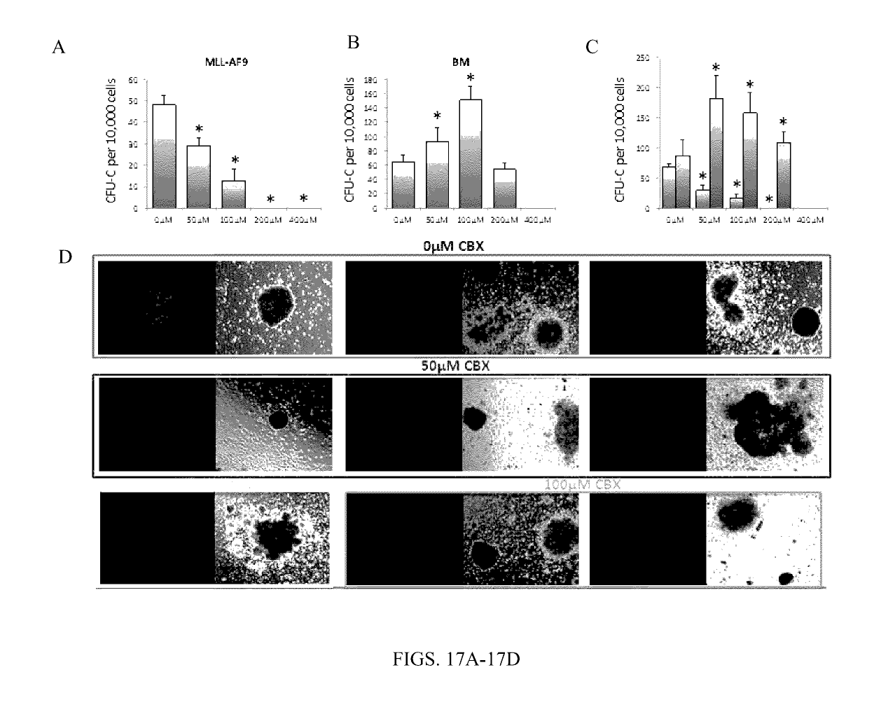

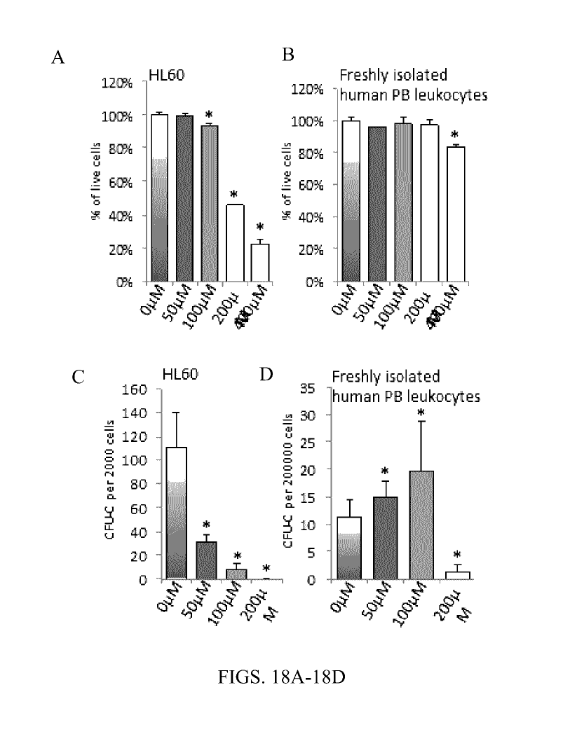

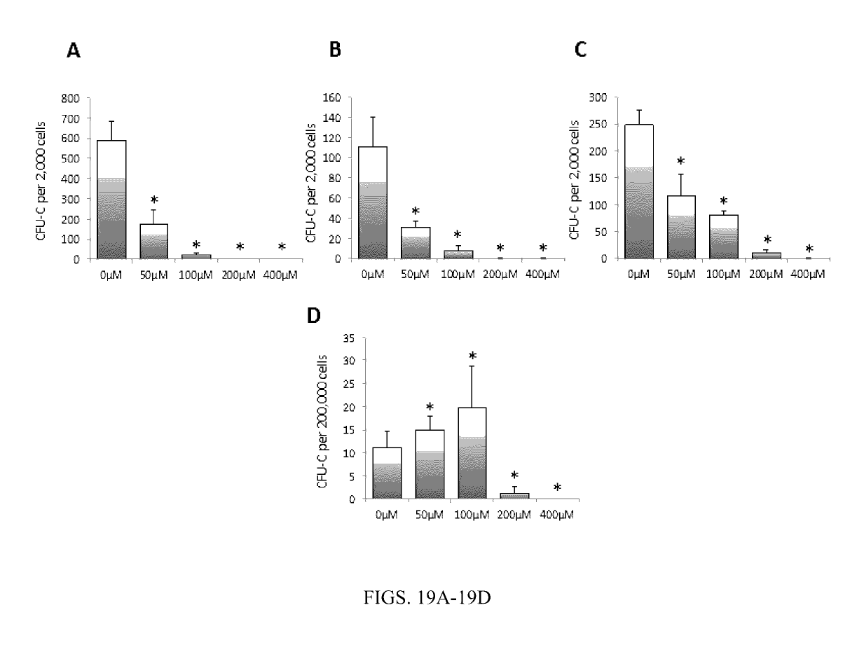

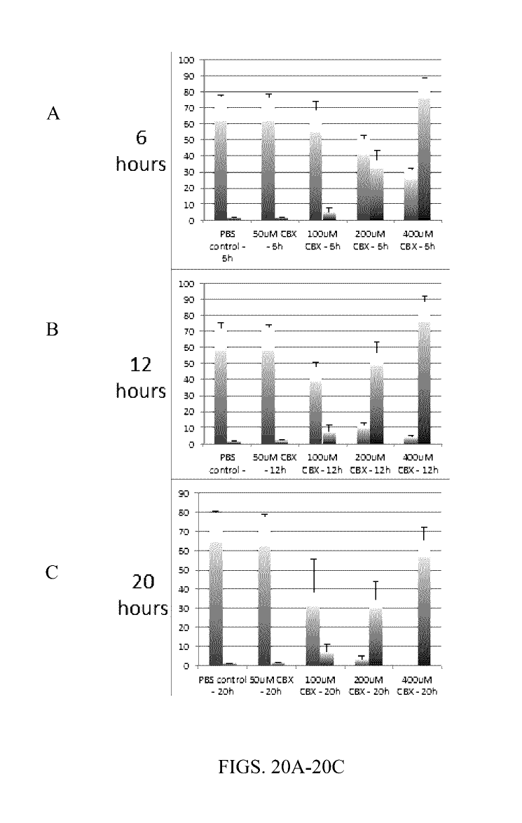

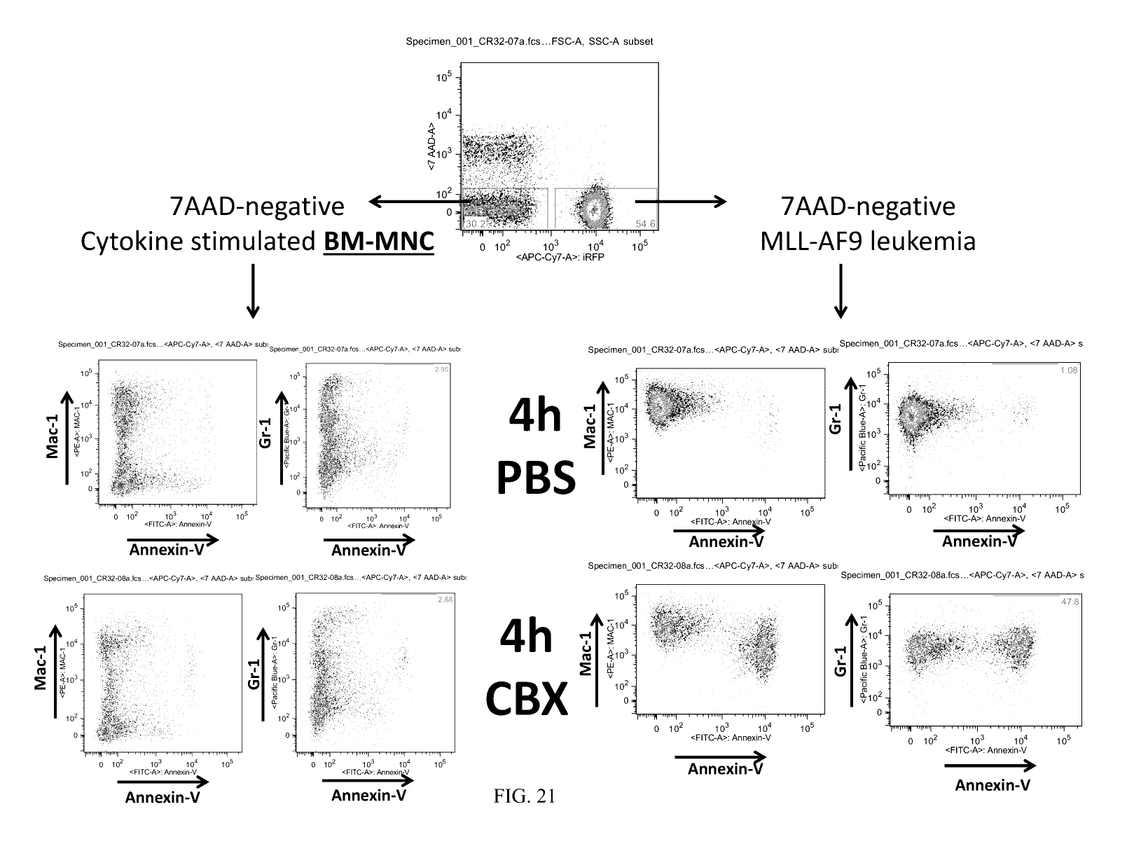

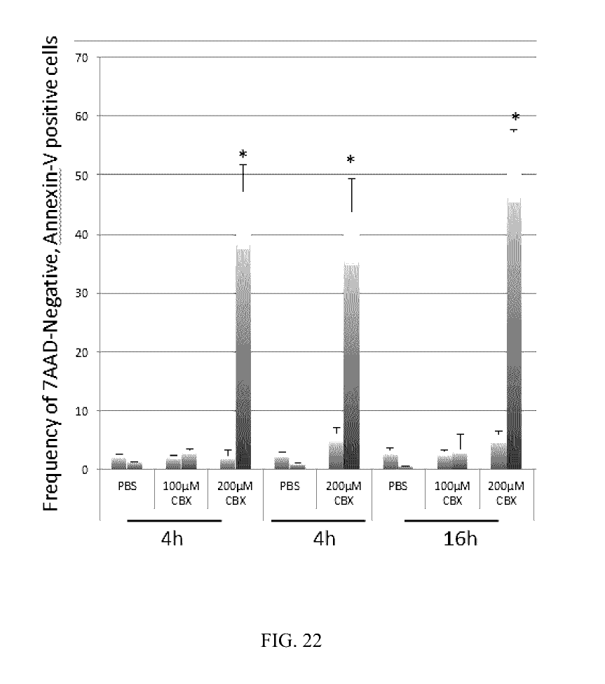

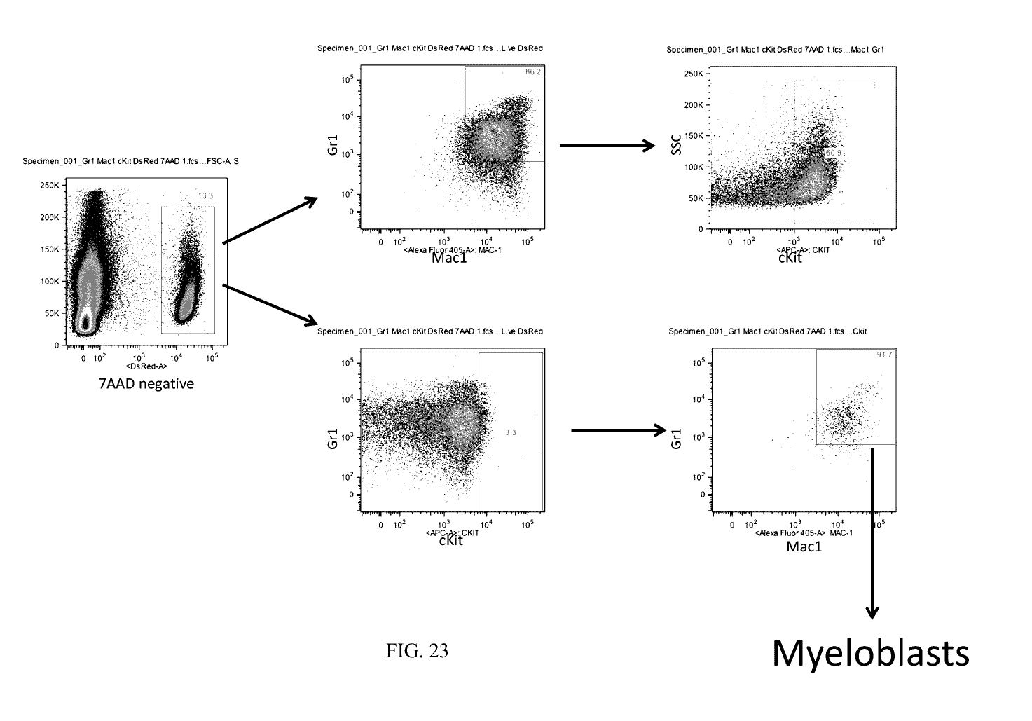

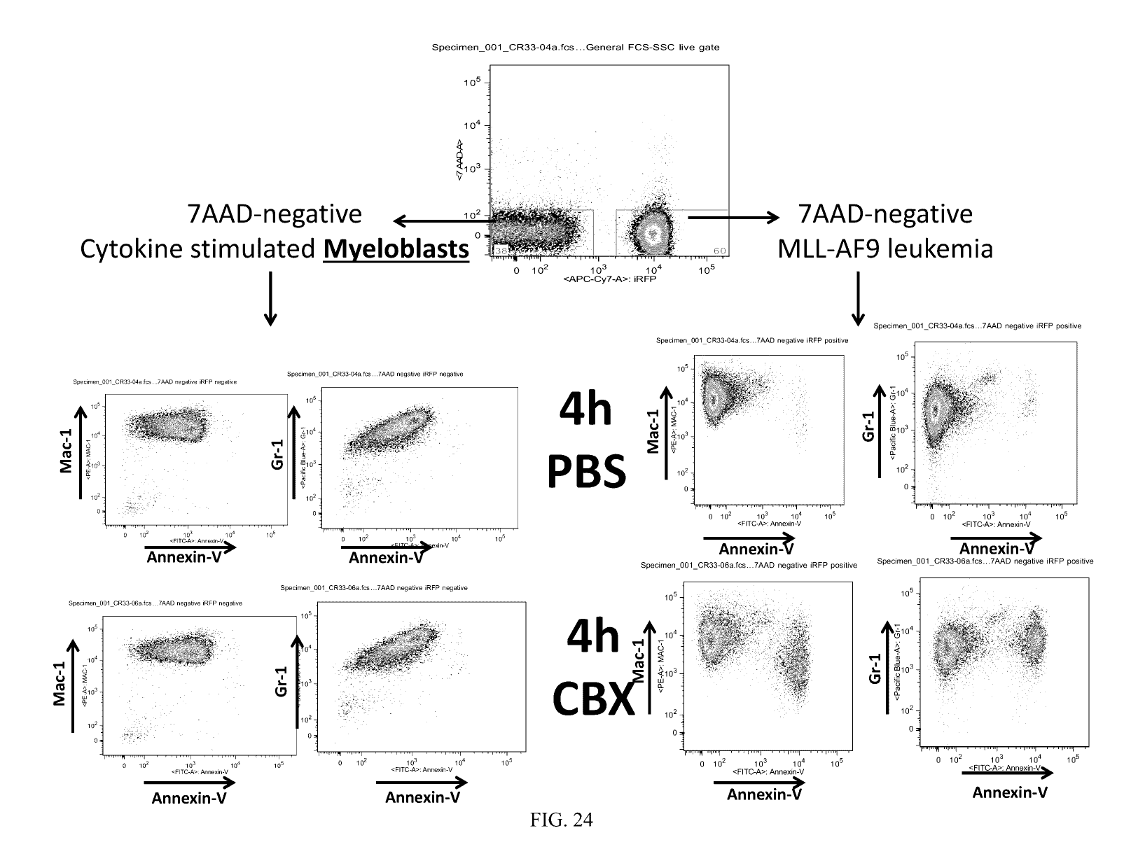

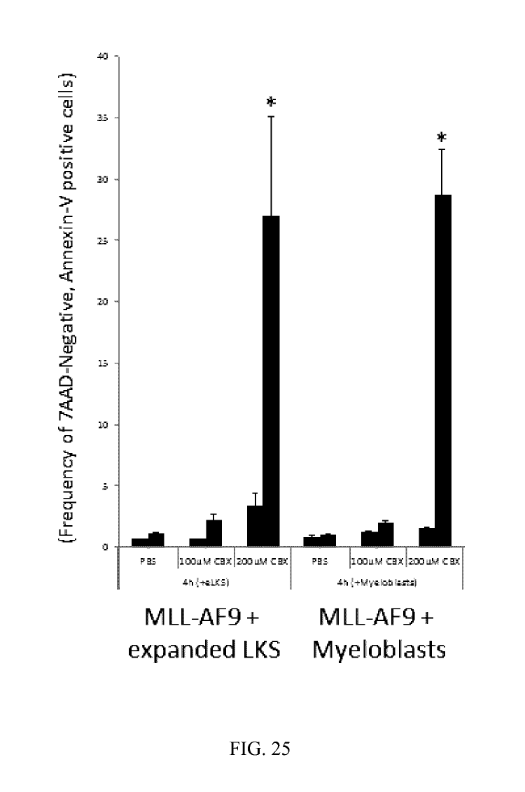

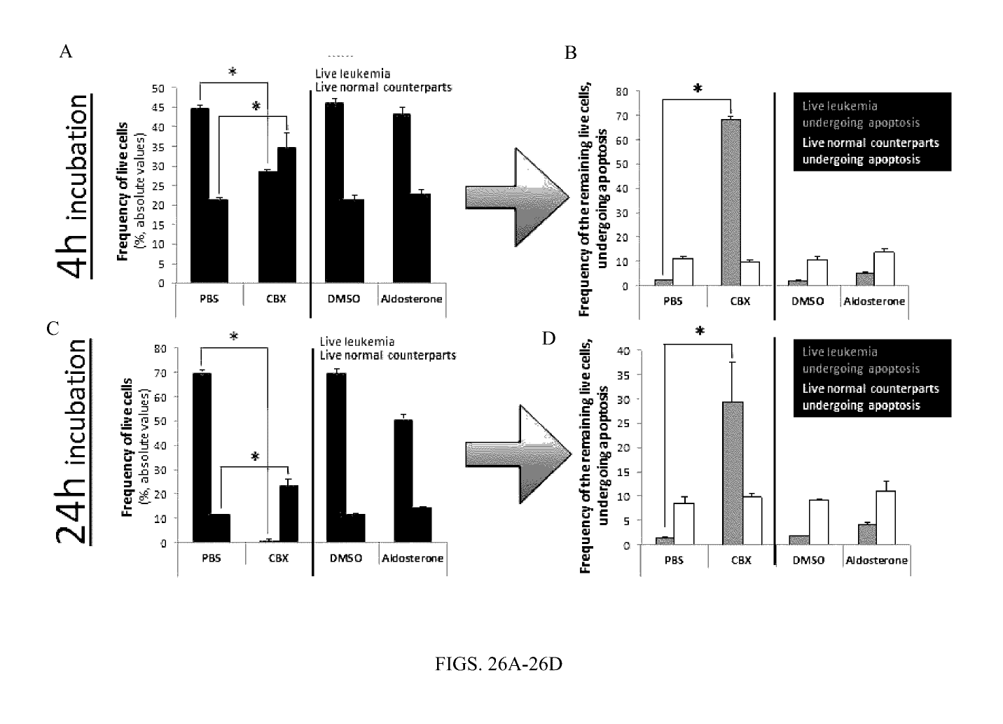

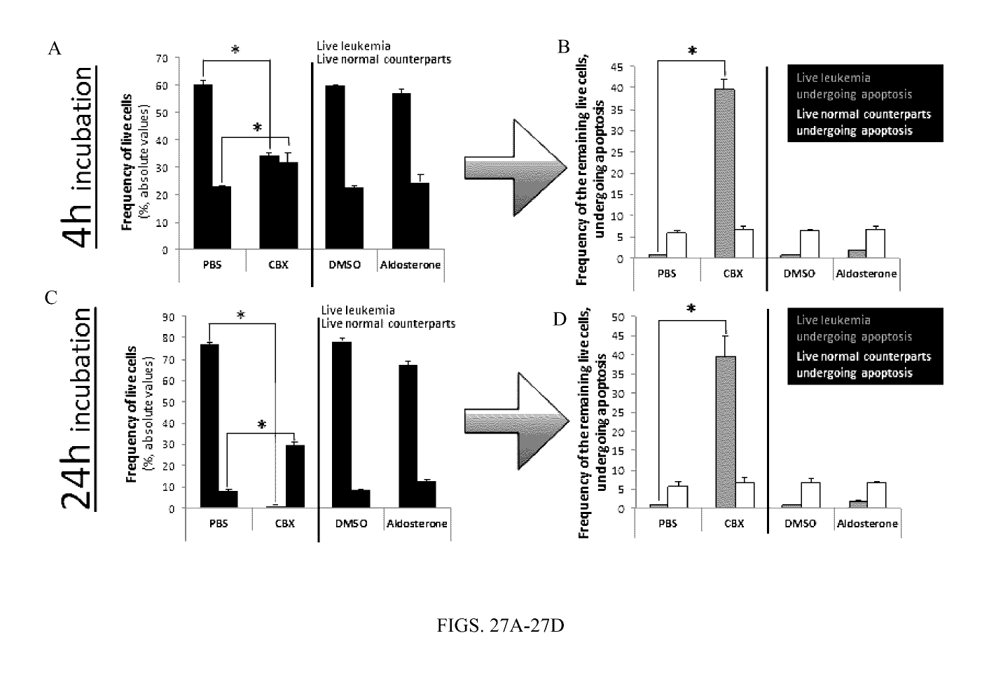

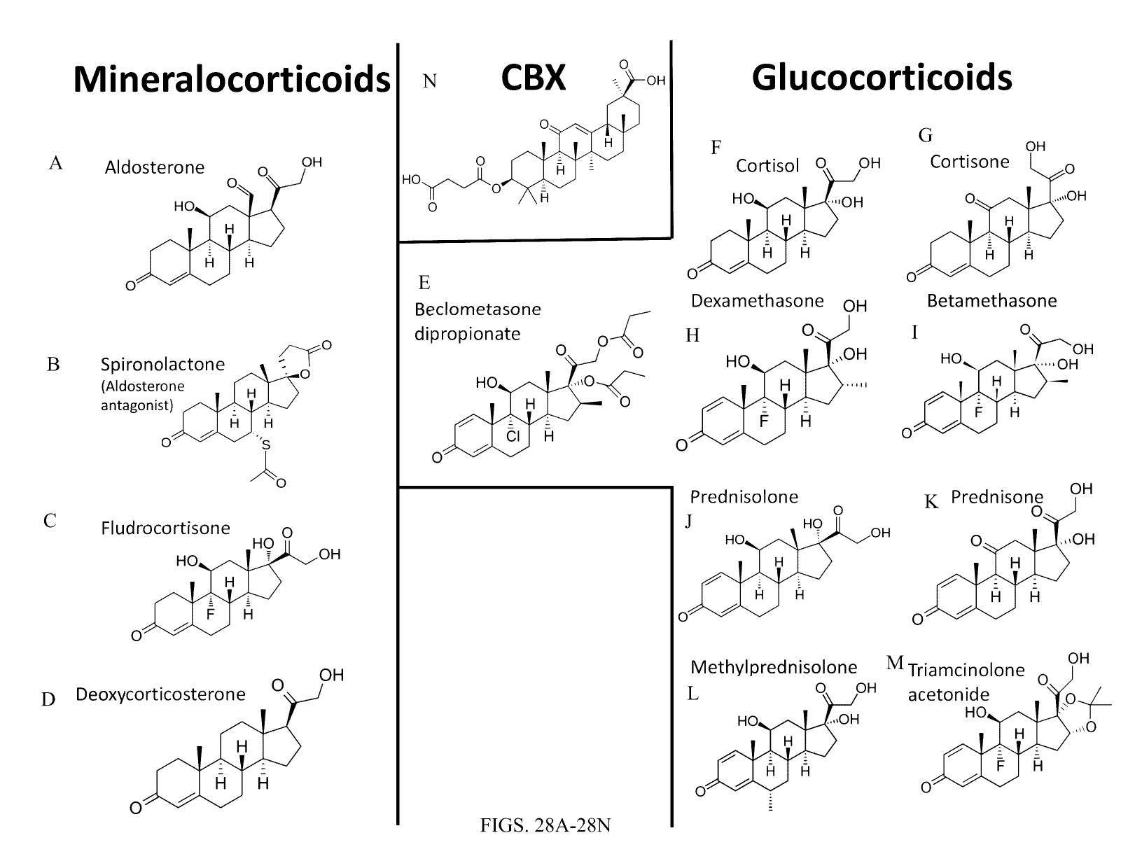

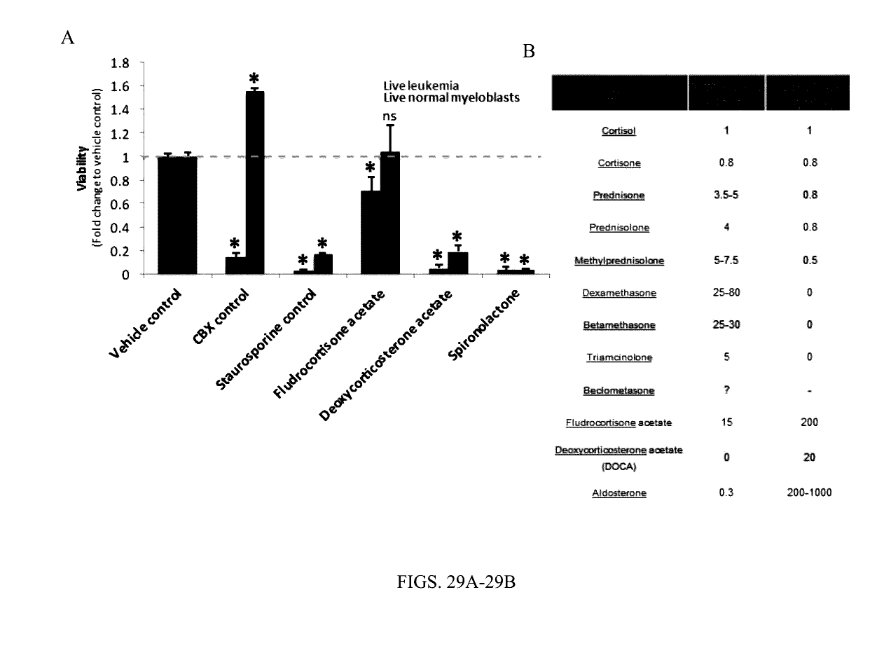

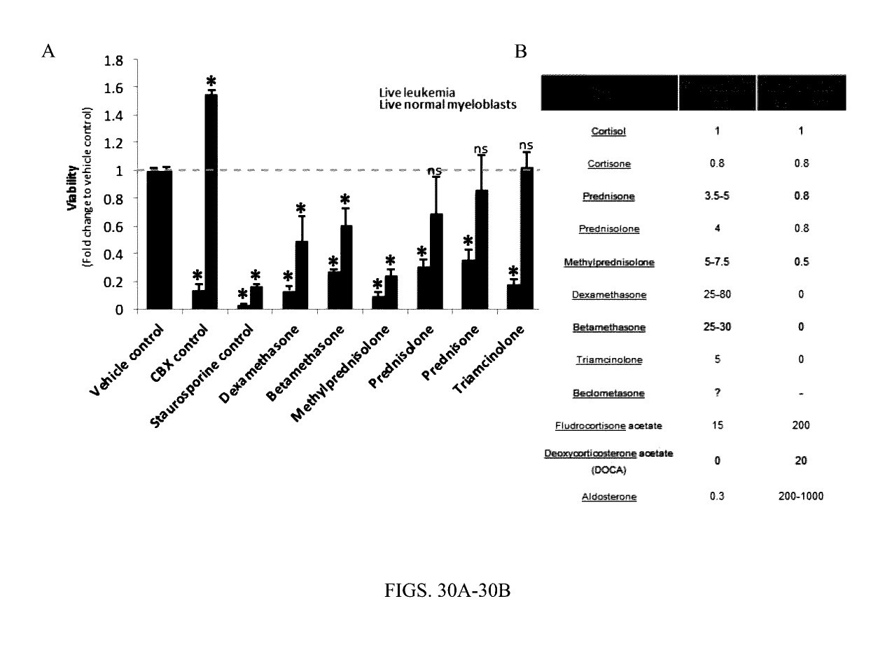

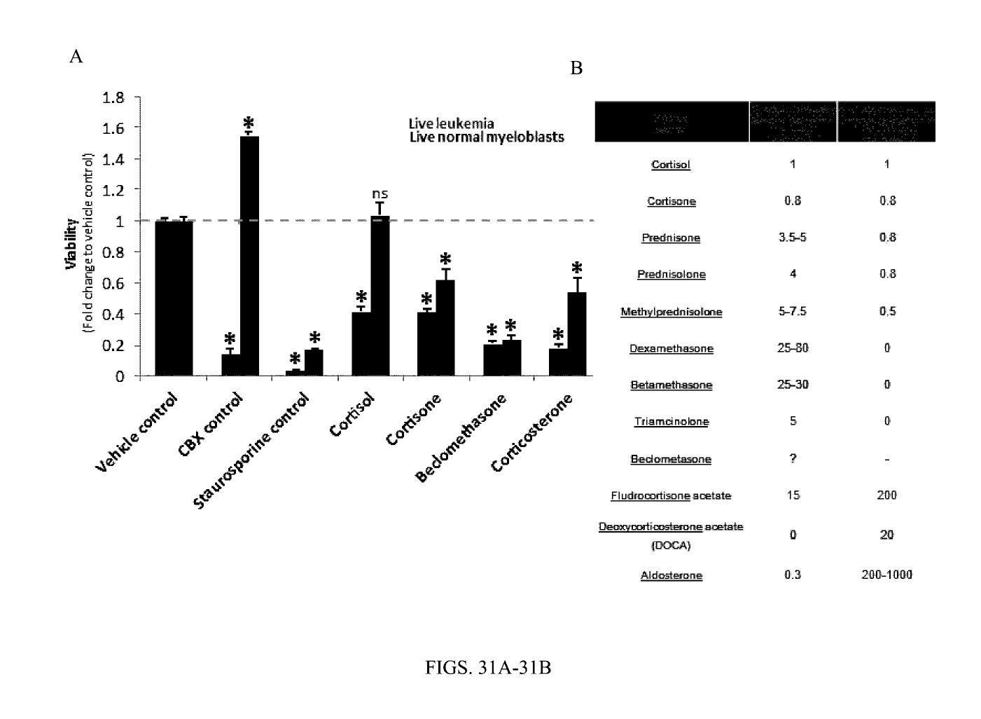

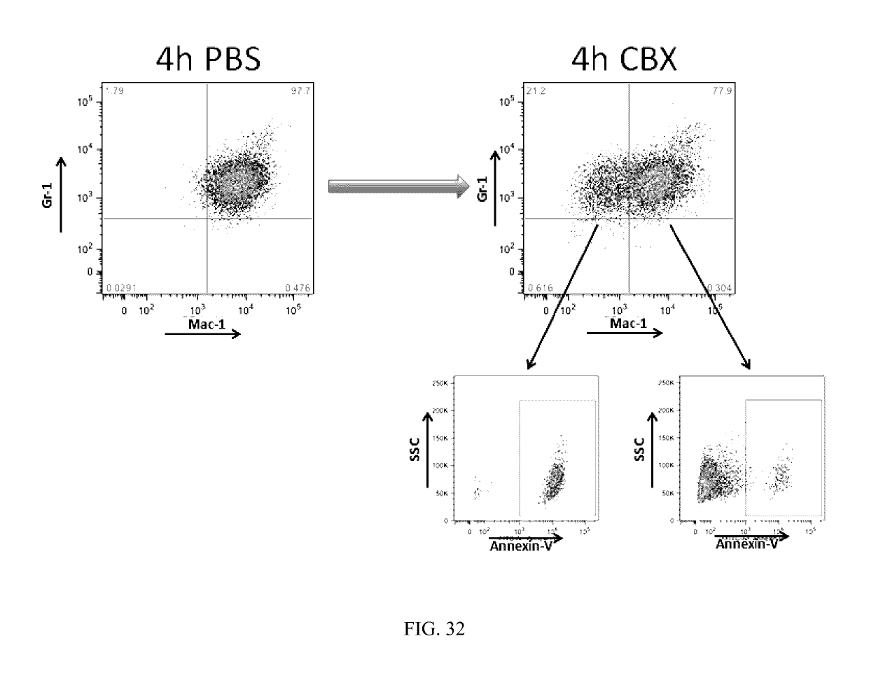

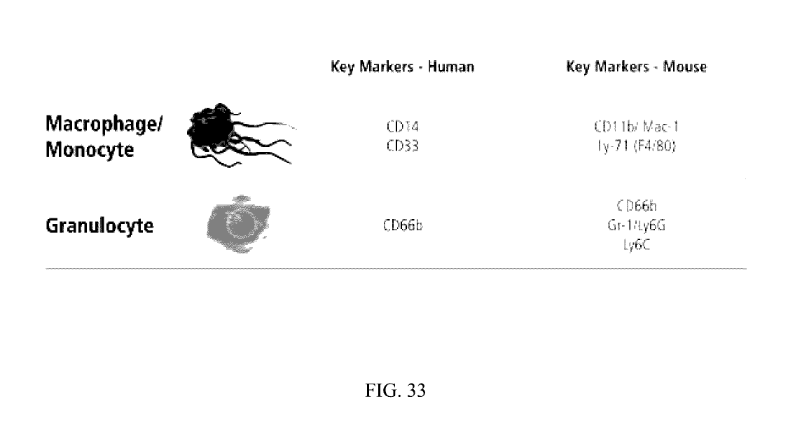

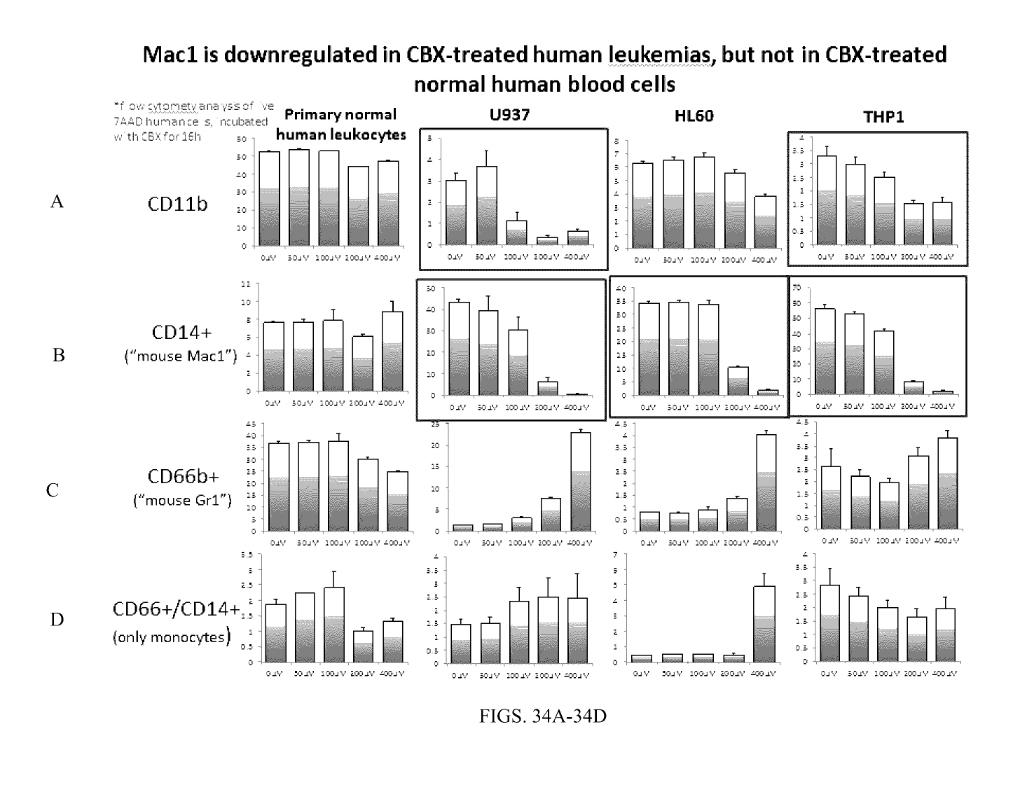

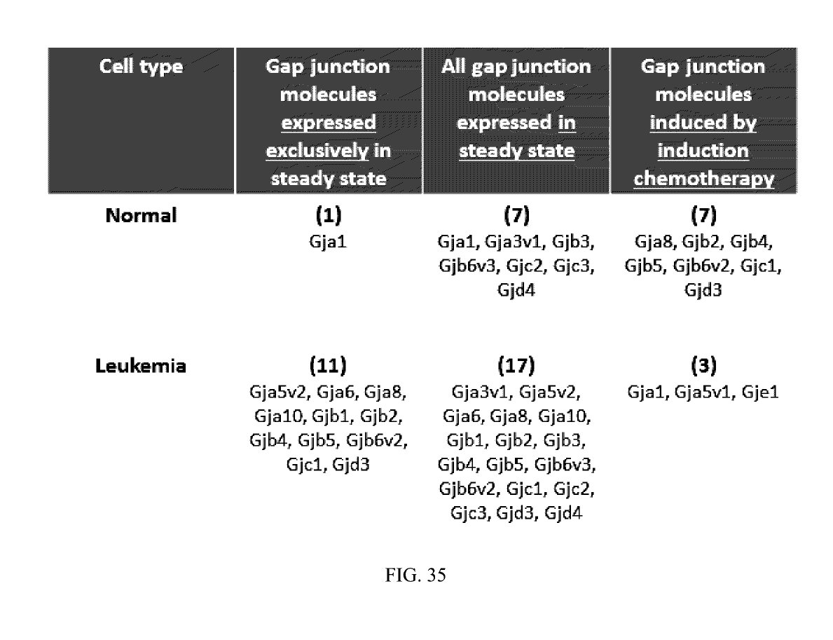

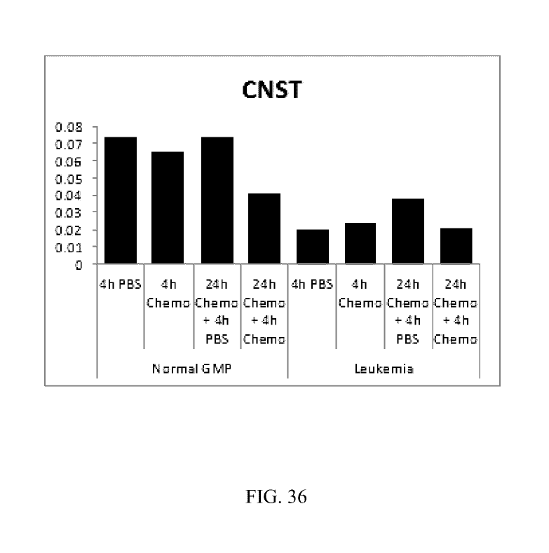

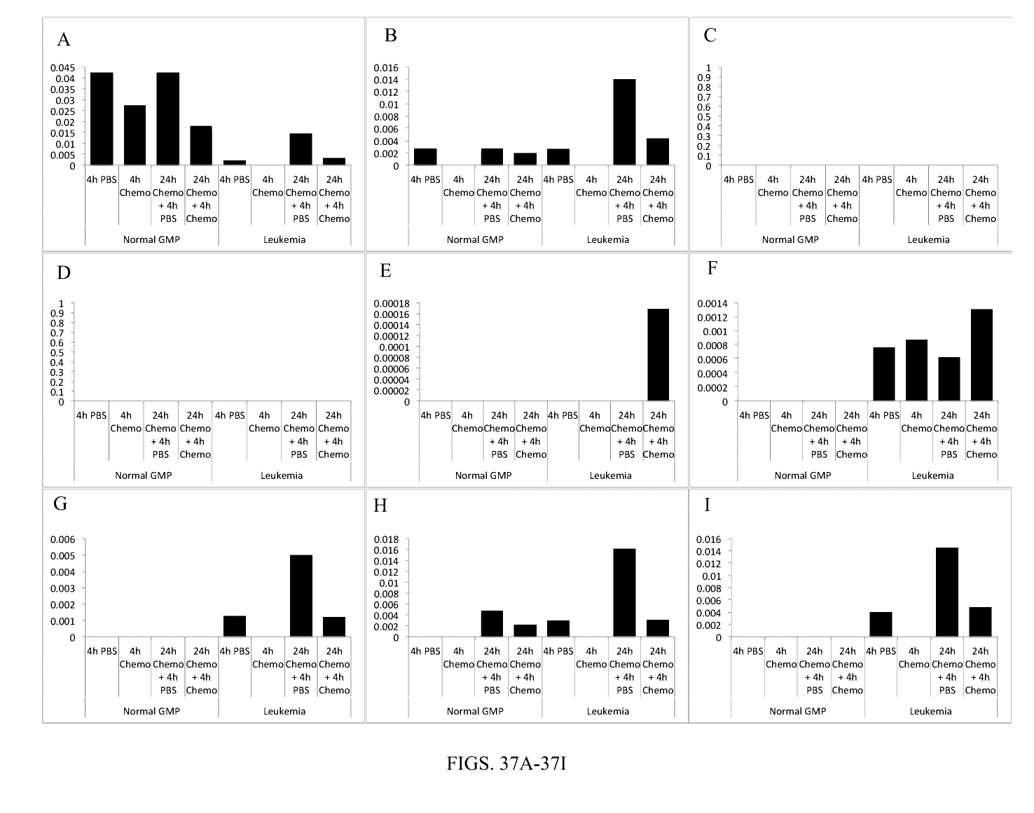

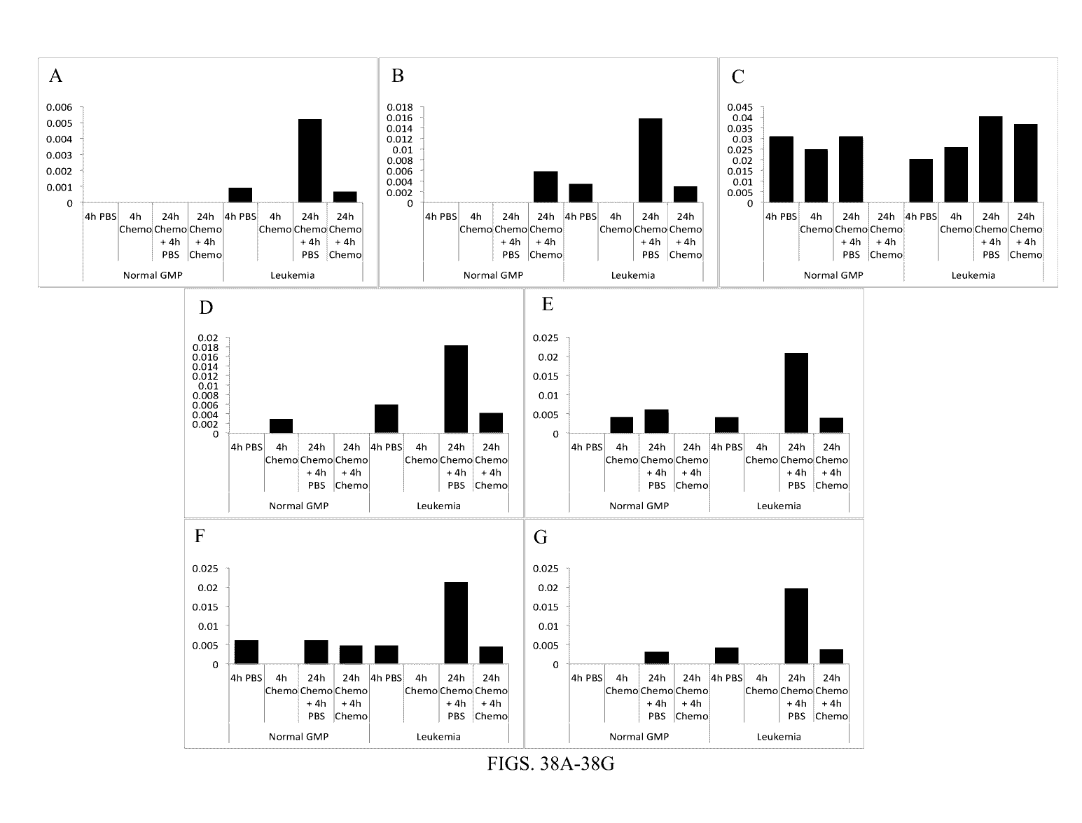

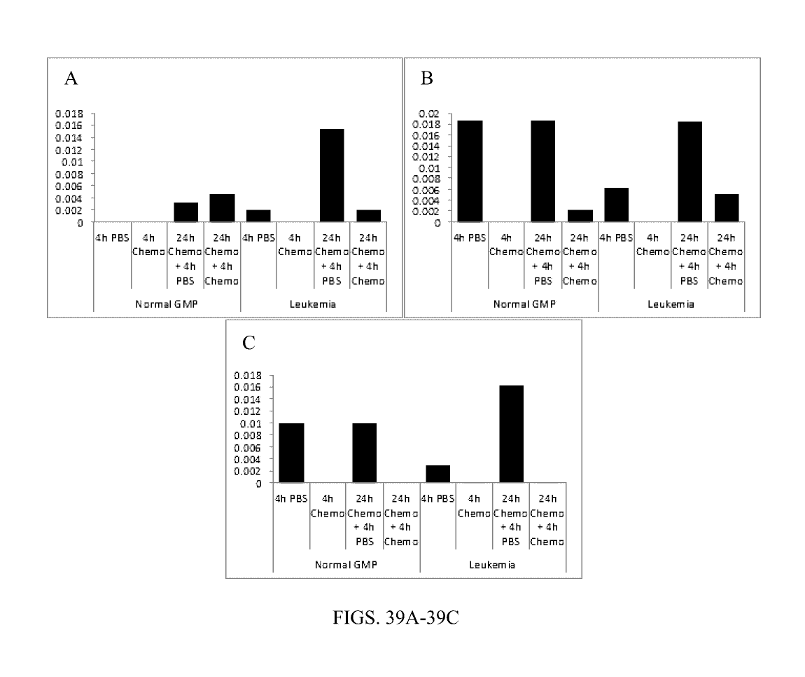

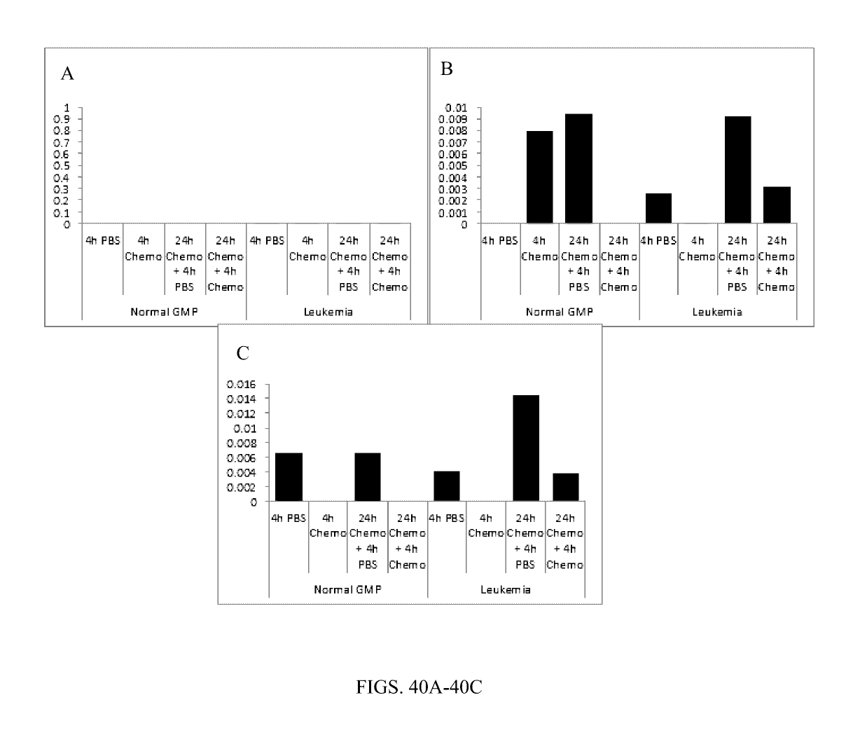

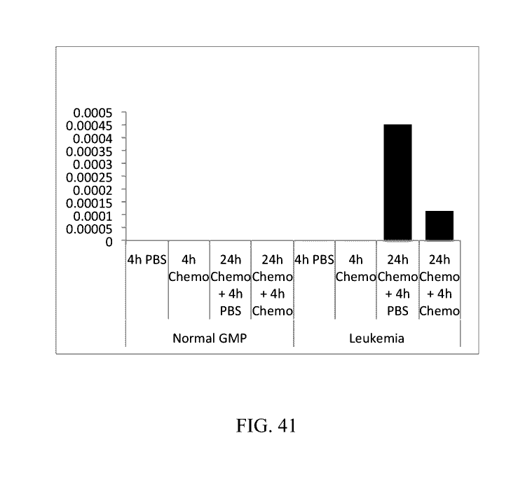

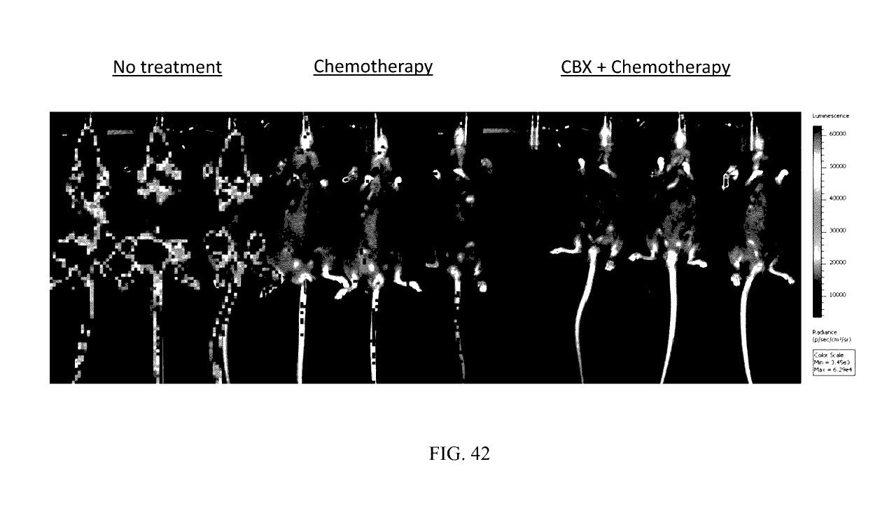

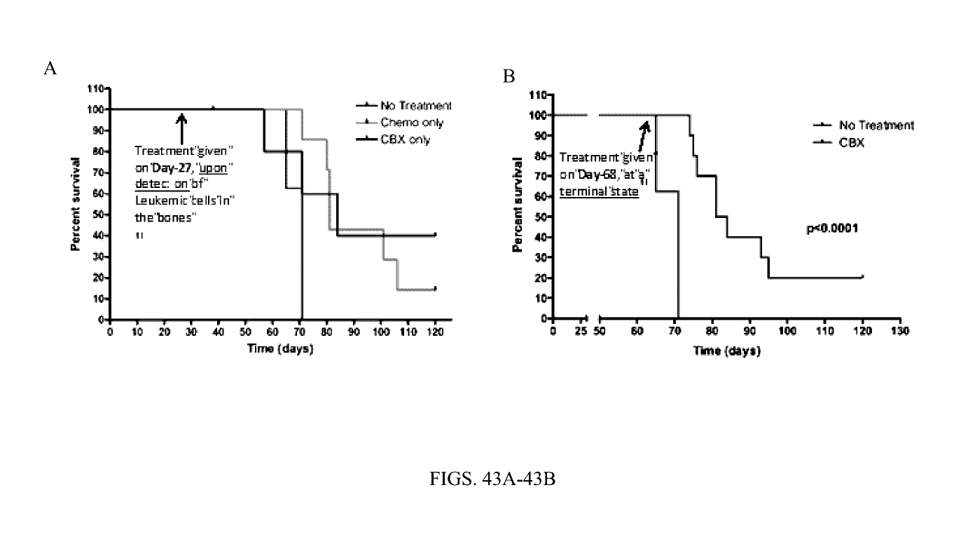

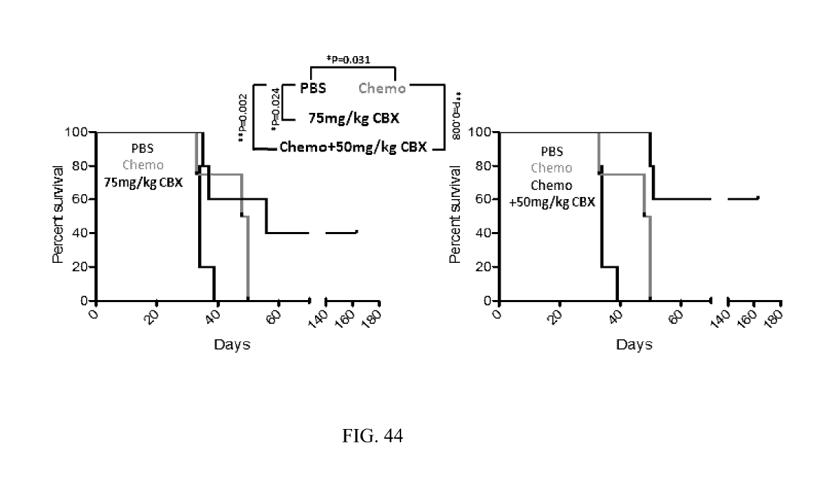

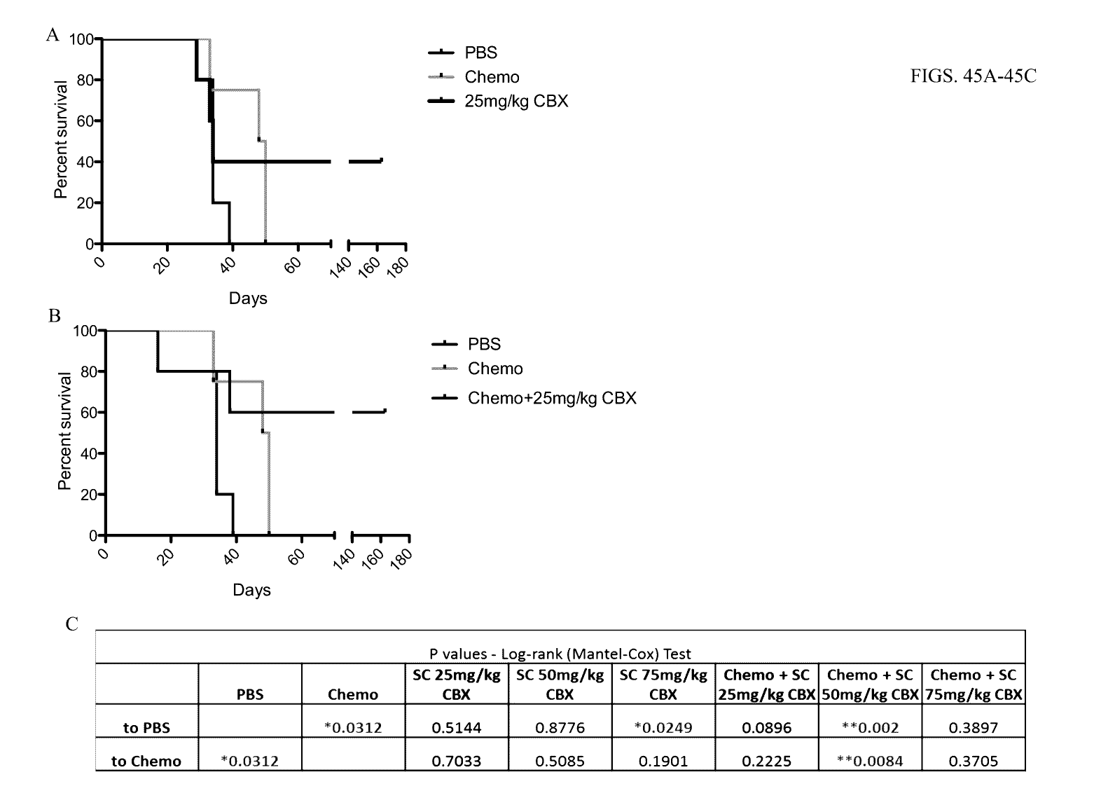

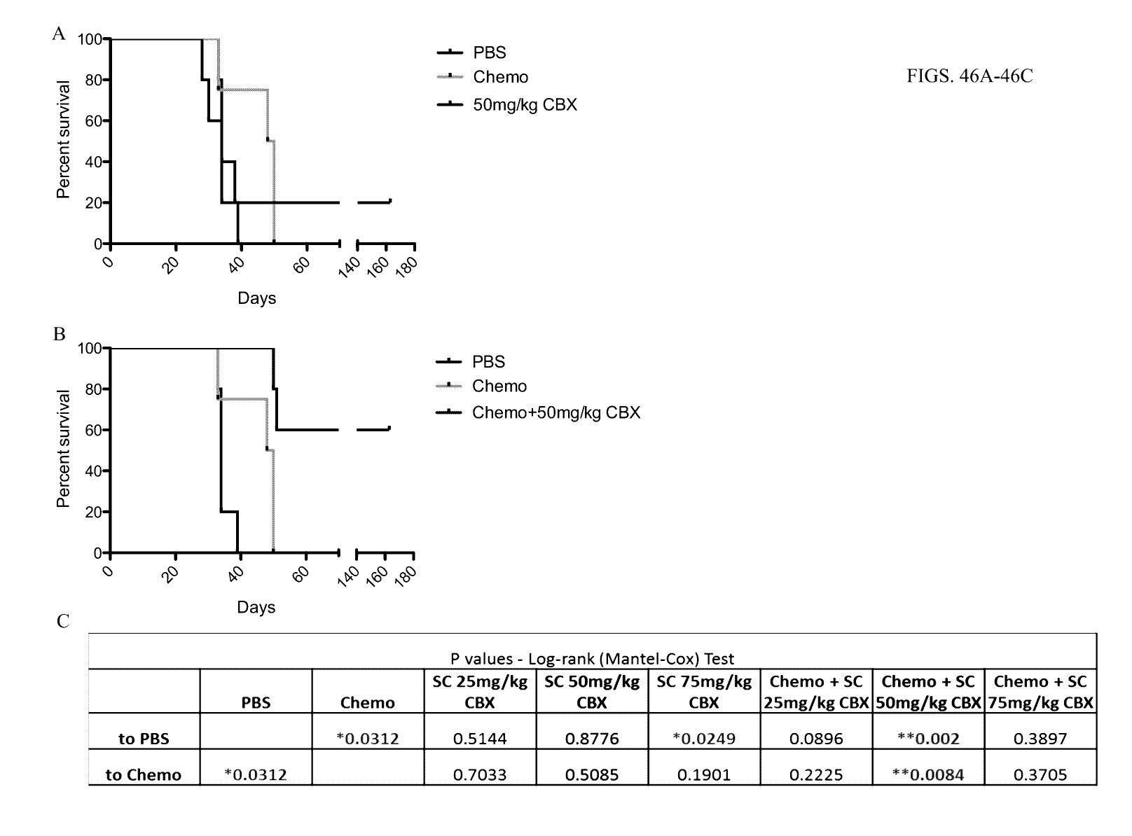

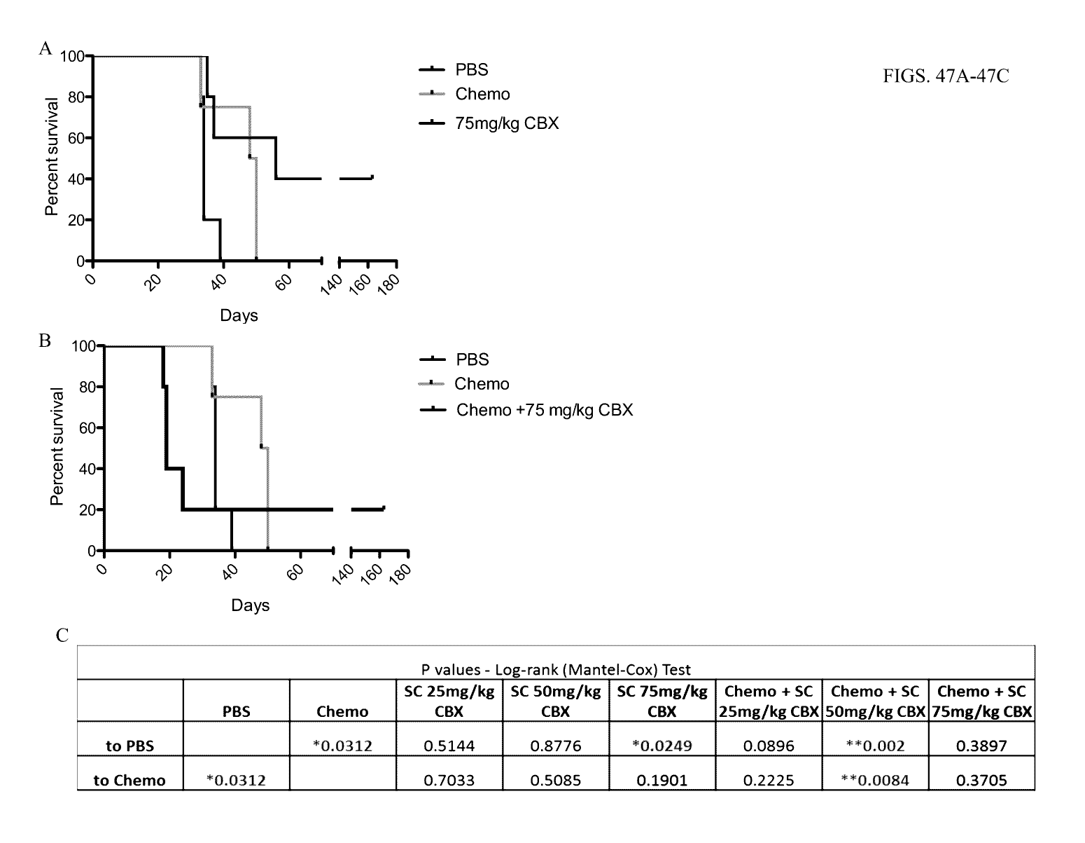

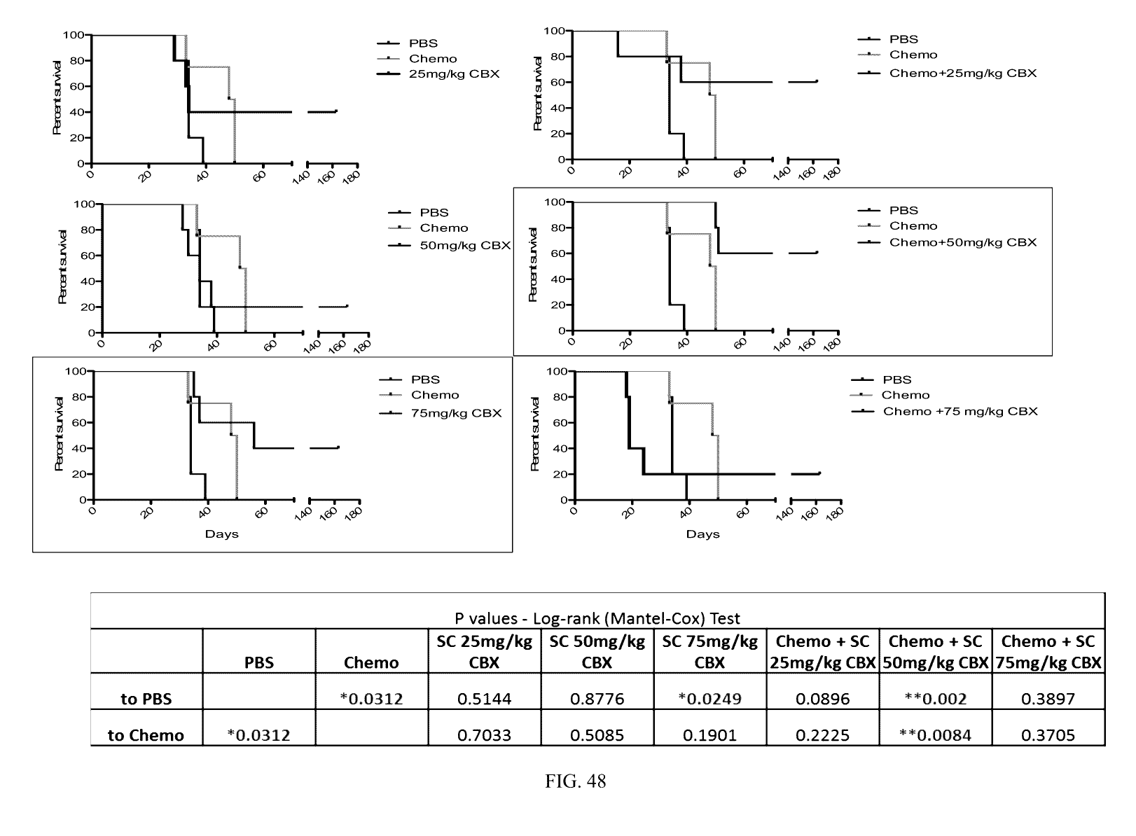

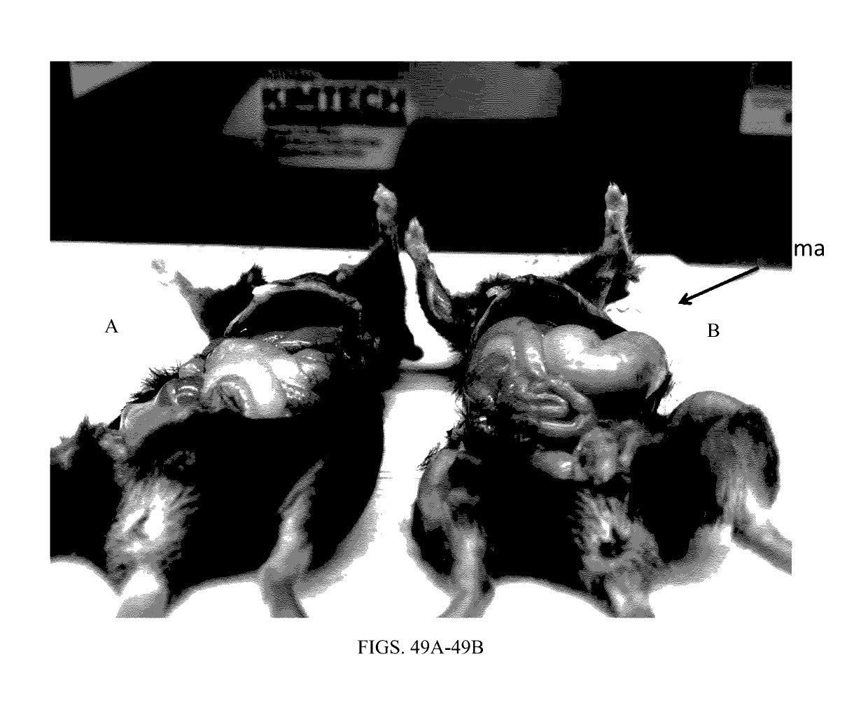

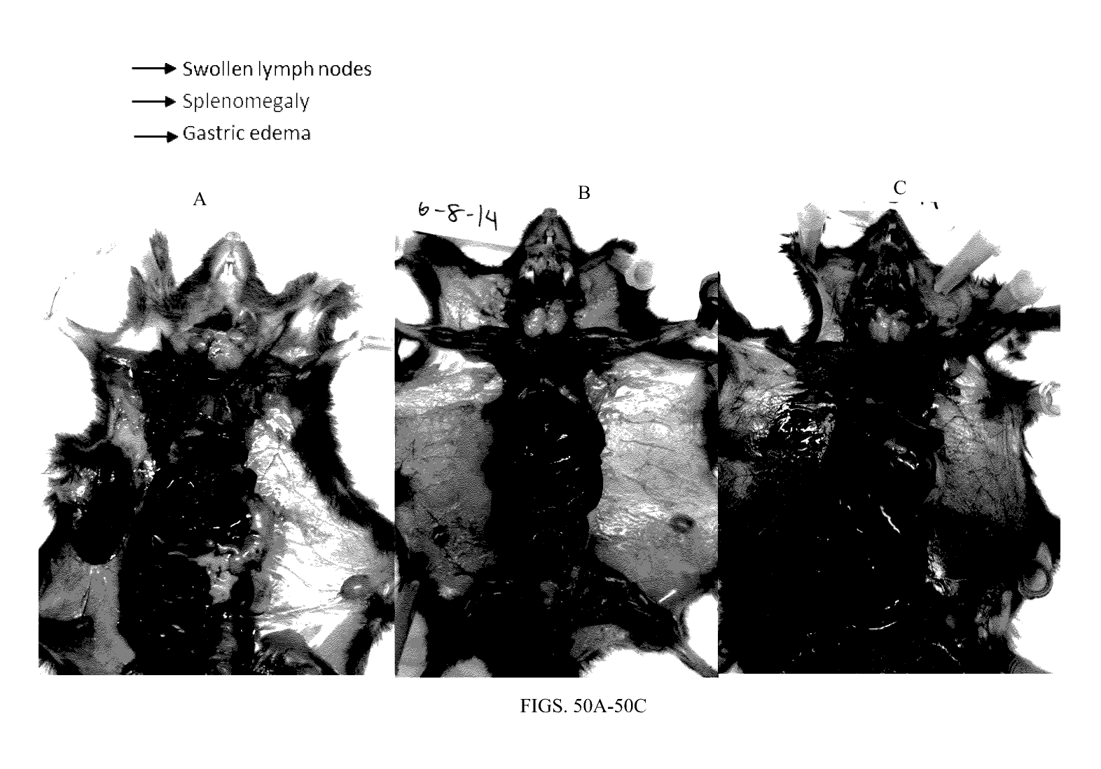

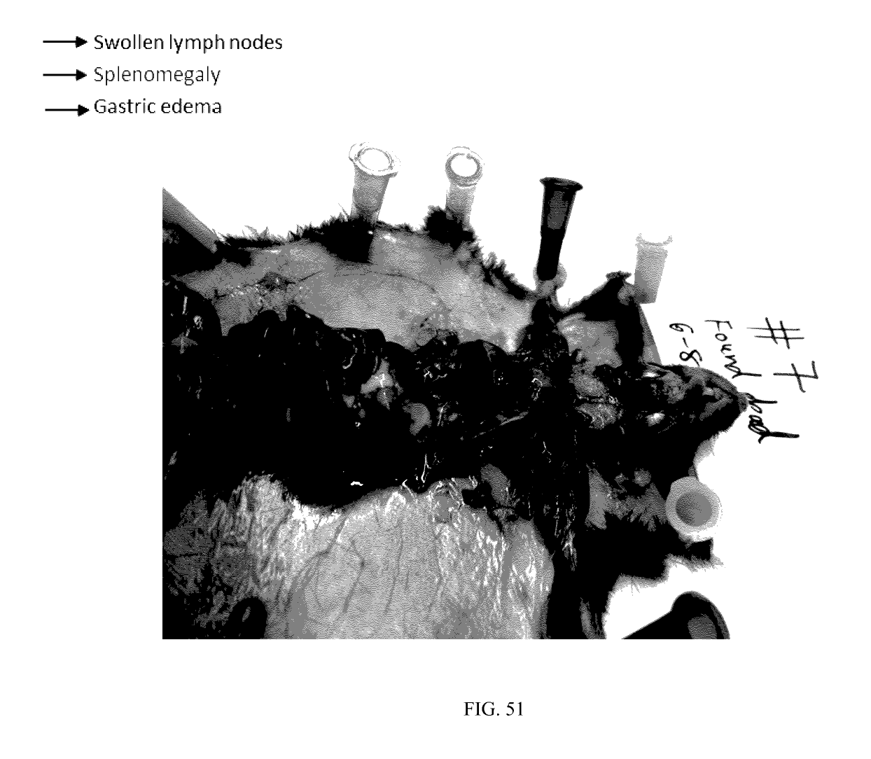

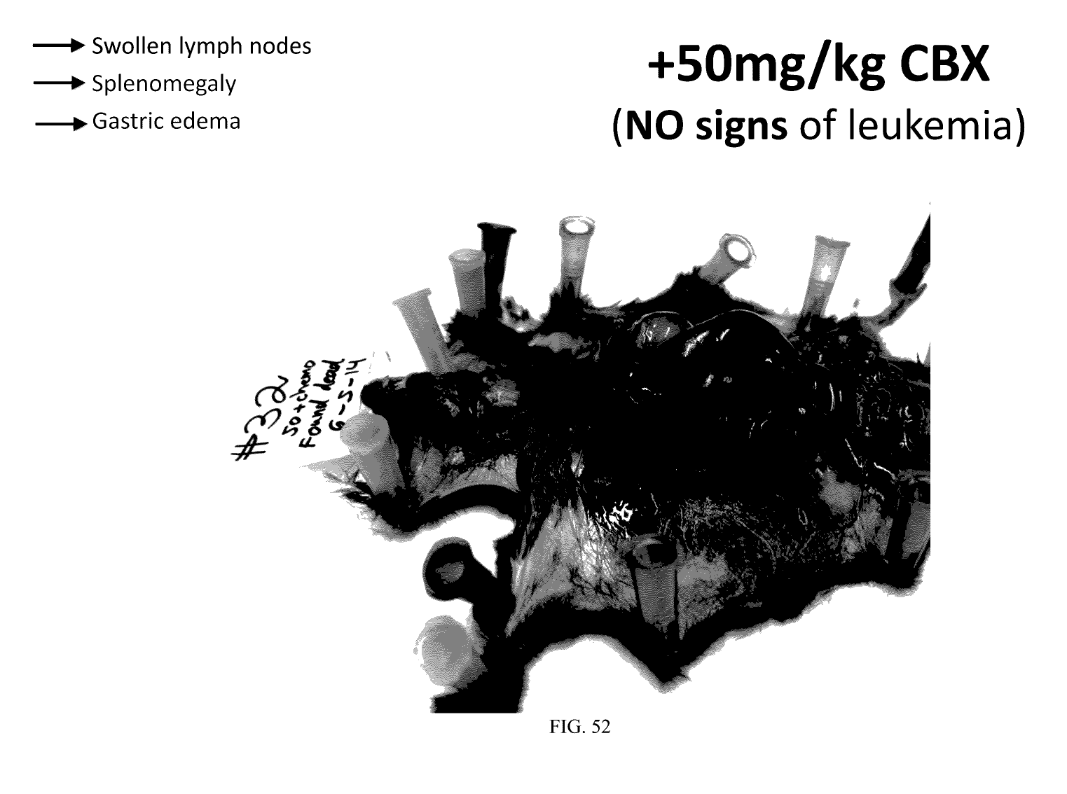

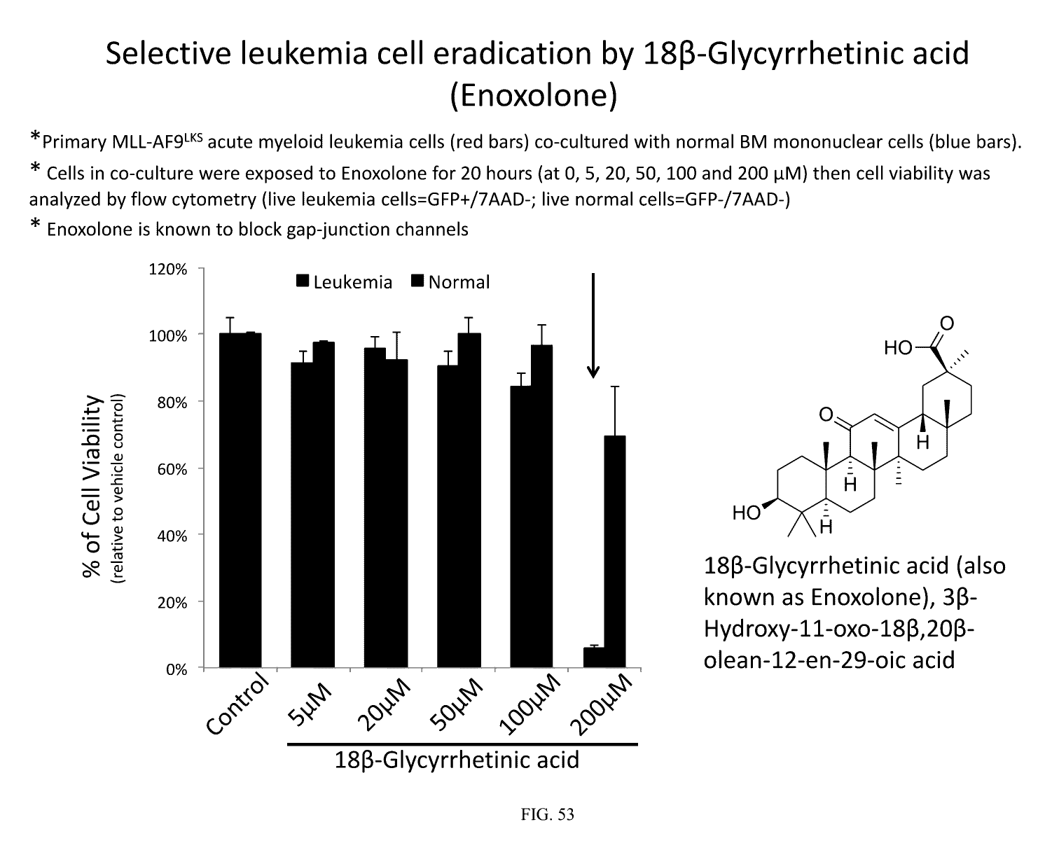

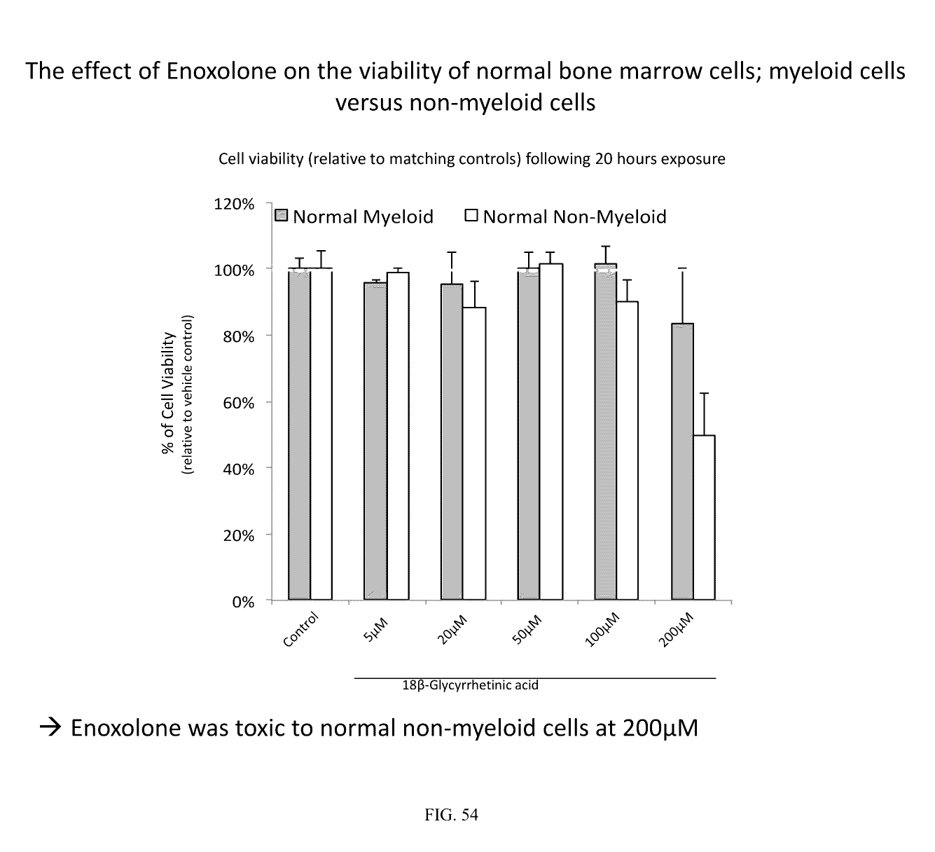

We then asked how blockage of gap junction intercellular communication affects AML cells, without additional treatment. To our surprise, primary MLL-AF9 cells were found to be sensitive to gap junction blockage and ˜20% of the cells were eradicated by 1001 μM CBX for 16 hours (FIG. 4A). Importantly, this was a leukemia-specific effect as non-leukemic, wild type primary leukocytes, freshly isolated from the BM or peripheral blood (PB) were not sensitive to CBX exposure even at high concentration of 2001M, whereas MLL-AF9 cell death was increased to ˜70% at 2001 μM (FIG. 4A). We further validated these results by mixing primary MLL-AF9 cells (expressing CD45.1 antigen) with freshly isolated normal BM leukocytes (expressing CD45.2 antigen), in a 1:1 ratio, and exposing the mixtures to increasing concentrations of CBX for 16 hours. These experiments confirmed that CBX selectively eradicates MLLAF9 cells without affecting the normal cells, even at concentrations as high as 400 μM (FIG. 4B). In order to exclude the possibility of clone specific effects, we have repeated the experiments with a different MLL-AF9 clone, generated independently, and also included HoxA9/Meis1 AML cells that represent a different type of AML. As depicted in FIGS. 4C and 4D, CBX selectively eradicated all AML cells tested, without affecting normal cells.

These results revealed an unexpected selective role of intercellular communication in maintaining leukemia cell survival. Since CBX has no cytotoxic effects on normal cells, we sought to understand the cause of death observed in the leukemic cells tested. MLL-AF9 AML cells are undifferentiated myeloid progenitors that express both Gr-1 and Mac-1 antigens, whereas mature myeloid cells express either Gr-1 (granulocytes) or Mac-1 (macrophages). We performed differentiation assays by testing the expression of Gr-1 and Mac-1 in primary MLL-AF9 cells after exposing them to 0 μM-2001M CBX for 16 hours. We found that indeed CBX is not toxic to the cells but instead induces their differentiation into mature granulocytes, which are short-lived cells.

### REFERENCES

1. Lane, S., Scadden, D. & Gilliland, D. The leukemic stem cell niche: current concepts and therapeutic opportunities. Blood 1150-1157 (2009). doi:10.1182/blood-2009-01-202606
2. Marcucci, G., Haferlach, T. & Dohner, H. Molecular genetics of adult acute myeloid leukemia: prognostic and therapeutic implications. Journal of clinical oncology: official journal of the American Society of Clinical Oncology 29, 475-86 (2011).
3. Pui, C.-H., Carroll, W. L., Meshinchi, S. & Arceci, R. J. Biology, risk stratification, and therapy of pediatric acute leukemias: an update. Journal of clinical oncology: official journal of the American Society of Clinical Oncology 29, 551-65 (2011).
4. Burnett, A., Wetzler, M. & Löwenberg, B. Therapeutic advances in acute myeloid leukemia. Journal of clinical oncology: official journal of the American Society of Clinical Oncology 29, 487-94 (2011).
5. Gillette, J. M., Larochelle, A., Dunbar, C. E. & Lippincott-Schwartz, J. Intercellular transfer to signalling endosomes regulates an ex vivo bone marrow niche. Nature cell biology 11, 303-11 (2009).
6. Walkley, C. R. et al. A microenvironment-induced myeloproliferative syndrome caused by retinoic acid receptor gamma deficiency. Cell 129, 1097-110 (2007).
7. Wei, J. et al. Microenvironment determines lineage fate in a human model of MLL-AF9 leukemia. Cancer cell 13, 483-95 (2008).
8. Huntly, B. J. P. et al. MOZ-TIF2, but not BCR-ABL, confers properties of leukemic stem cells to committed murine hernatopoietic progenitors. Cancer cell 6, 587-96 (2004).
9. Cozzio, A. et al. Similar MLL-associated leukemias arising from self-renewing stem cells and short-lived myeloid progenitors. Genes & development 17, 3029-35 (2003).
10. So, C. W. et al. MLL-GAS7 transforms multipotent hematopoietic progenitors and induces mixed lineage leukemias in mice. Cancer cell 3, 161-71 (2003).
11. Krivtsov, A. V et al. Transformation from committed progenitor to leukaemia stem cell initiated by MLL-AF9. Nature 442, 818-22 (2006).
12. Sykes, S. M. et al. AKT/FOXO signaling enforces reversible differentiation blockade in myeloid leukemias. Cell 146, 697-708 (2011).
13. Schoch, C. et al. AML with 11q23/MLL abnormalities as defined by the WHO classification: incidence, partner chromosomes, FAB subtype, age distribution, and prognostic impact in an unselected series of 1897 cytogenetically analyzed AML cases. Blood 102, 2395-402 (2003).
14. Zuber, J. et al. Mouse models of human AML accurately predict chemotherapy response. Genes & development 23, 877-89 (2009).
15. Pardee, T. S., Zuber, J. & Lowe, S. W. Flt3-ITD alters chemotherapy response in vitro and in vivo in a p53-dependent manner. Experimental hematology 39, 473-485.e4 (2011).
16. Itoh, K. et al. Reproducible establishment of hemopoietic supportive stromal cell lines from murine bone marrow. Experimental Hematology 21, 145-153 (1989).
17. Schajnovitz, A. et al. CXCL12 secretion by bone marrow stromal cells is dependent on cell contact and mediated by connexin-43 and connexin-45 gap junctions. Nature immunology 12, 391-8 (2011).
18. Sipkins, D. a et al. In vivo imaging of specialized bone marrow endothelial microdomains for tumour engraftment. Nature 435, 969-73 (2005).
19. Lo Celso, C. et al. Live-animal tracking of individual haematopoietic stem/progenitor cells in their niche. Nature 457, 92-6 (2009).
20. Fujisaki, J. et al. In vivo imaging of Treg cells providing immune privilege to the haematopoietic stem-cell niche. Nature 474, 216-9 (2011).
21. Nuotio-Antar, A. M., Hachey, D. L. & Hasty, A. H. Carbenoxolone treatment attenuates symptoms of metabolic syndrome and atherogenesis in obese, hyperlipidemic mice. American journal of physiology. Endocrinology and metabolism 293, E1517-28 (2007).
22. Davies, G. J., Rhodes, J. & Calcraft, B. J. Complications of Carbenoxolone Therapy. British medical journal 3, 400, 401-402 (1974).

1-102. (canceled)

103. A method of eradicating leukemic cells in a population of cells, the method comprising contacting the population of cells with an effective amount of a glycyrrhetinic acid derivative, thereby eradicating leukemic cells in the cell population.

104. The method of claim 103, wherein the glycyrrhetinic acid derivative is selected from the group consisting of glycyrrhizine, glycyrrhizinic acid, 18-β-glycyrrhetinic acid, carbenoxolone, and 2-hydroxyethyl-18β-glycyrrhetinic acid amide.

105. The method of claim 103, wherein the glycyrrhetinic acid derivative comprises carbenoxolone or an analog thereof.

106. The method of claim 103, wherein the leukemic cells comprise an acute myeloid leukemia cell line selected from the group consisting of MLL-AF9 cells, KG-1 cells, KG-1a cells, U937 cells, HL60 cells, NB-4 cells, and THP1 cells.

107. The method of claim 103, wherein the contact is in a human subject.

108. The method of claim 107, wherein the subject suffers from leukemia.

109. The method of claim 107, wherein the subject suffers from acute myeloid leukemia.

110. The method of claim 107, wherein the glycyrrhetinic acid derivative selectively eradicates leukemic cells in the subject without eradicating normal leukocytes in the subject.

111. A method of treating acute myeloid leukemia in a subject in need thereof, the method comprising administering to the subject an effective amount of a glycyrrhetinic acid derivative, thereby treating acute myeloid leukemia in the subject.

112. The method of claim 111, wherein the glycyrrhetinic acid derivative is selected from the group consisting of glycyrrhizine, glycyrrhizinic acid, 18-β-glycyrrhetinic acid, carbenoxolone, and 2-hydroxyethyl-18β-glycyrrhetinic acid amide.

113. The method of claim 111, wherein the glycyrrhetinic acid derivative comprises carbenoxolone or an analog thereof.

114. The method of claim 111, wherein the glycyrrhetinic acid derivative selectively eradicates leukemic cells in the subject without eradicating normal leukocytes in the subject.

115. The method of claim 111, further comprising administering an induction chemotherapy treatment regimen to the subject.

116. The method of claim 111, wherein the subject is suffering from refractory or relapsed acute myeloid leukemia.

117. A method of promoting survival of a subject suffering from acute myeloid leukemia, the method comprising administering to the subject an effective amount of a glycyrrhetinic acid derivative, thereby promoting survival of the subject.

118. The method of claim 117, wherein the glycyrrhetinic acid derivative is selected from the group consisting of glycyrrhizine, glycyrrhizinic acid, 18-β-glycyrrhetinic acid, carbenoxolone, and 2-hydroxyethyl-18β-glycyrrhetinic acid amide.

119. The method of claim 117, wherein the glycyrrhetinic acid derivative comprises carbenoxolone or an analog thereof.

120. The method of claim 117, wherein the subject is suffering from refractory or relapsed acute myeloid leukemia.

121. The method of claim 117, wherein the glycyrrhetinic acid derivative selectively eradicates leukemic cells in the subject without eradicating normal leukocytes in the subject.

122. The method of claim 117, further comprising administering an induction chemotherapy treatment regimen to the subject.
